(19) United States
(12) Patent Application Publication
(10) Pub. No.: US 2015/0010928 A1
(43) Pub. Date: Jan. 8, 2015

(54) MEANS AND METHODS FOR ASSESSING AN ENDOCRINE DISEASE OR DISORDER

(71) Applicant: BASF SE, Ludwigshafen (DE)

(72) Inventors: Tilmann B. Walk, Kleinmachnow (DE); Bennard van Ravenzwaay, Altrip (DE); Werner Mellert, Hassloch (DE); Eric Fabian, Speyer (DE); Volker Strauss, Bad Durkheim (DE); Hennicke Kamp, Bischheim (DE); Jan C. Wiemer, Berlin (DE); Ralf Looser, Berlin (DE); Michael Manfred Herold, Berlin (DE); Alexandre Prokoudine, Berlin (DE)

(73) Assignee: BASF SE, Ludwigshafen (DE)

(21) Appl. No.: 14/376,921

(22) PCT Filed: Feb. 15, 2013
(86) PCT No.: PCT/IB2013/051224

§ 371 Date: Aug. 6, 2014

Related U.S. Application Data
(60) Provisional application No. 61/598,965, filed on Feb. 15, 2012.

(30) Foreign Application Priority Data
Feb. 15, 2012 (EP) 12155640.1

(51) Int. Cl. G01N 33/68

(52) U.S. Cl. CPC G01N 33/6893; G01N 2800/04; G01N 2500/20
USPC 435/7.92; 435/287.2

(57) ABSTRACT

The present invention pertains to the field of diagnostics for an endocrine disease or disorder and assessments for risk stratification of chemical compounds. Specifically, it relates to a method for diagnosing an endocrine disease or disorder. It also relates to a method for determining whether a compound is capable of inducing such an endocrine disease or disorder in a subject and to a method of identifying a drug for treating an endocrine disease or disorder. Furthermore, the present invention relates to a device and a kit for diagnosing an endocrine disease or disorder.

The present invention pertains to the field of diagnostics for an endocrine disease or disorder and assessments for risk stratification of chemical compounds. Specifically, it relates to a method for diagnosing an endocrine disease or disorder. It also relates to a method for determining whether a compound is capable of inducing such an endocrine disease or disorder in a subject and to a method of identifying a drug for treating an endocrine disease or disorder. Furthermore, the present invention relates to a device and a kit for diagnosing an endocrine disease or disorder.

The endocrine system is involved in various physiological functions of an organism. In particular, it controls body homeostasis and thereby influences metabolism, growth and development, organ or tissue functions such as cardiovascular functions or kidney functions, as well as mood. The endocrine system comprises the so-called endocrine glands that secrete hormones into the blood in order to affect the body tissues or organs. Diseases or disorders of the endocrine system are usually characterized by improper hormone production and/or release or by impaired responsiveness to hormones of effector tissues. Particular endocrine organs or tissues as well as endocrine diseases and disorders are further specified below.

The adrenal glands in mammals are flattened bi-lobed organs located in close proximity to the kidneys. The adrenal glands receive arterial blood from branches of the aorta or from the phrenic, renal, and lumbar arteries resulting in a subcapsular sinusoidal vascular plexus that drains through the cortex into the medulla. The cortex is histologically characterized by defined regions or zones. The cortical zones consist of the zona glomerulosa (multiformis), zona fasciculata, and zona reticularis. The mineralocorticoid-producing zona glomerulosa (multiformis) comprised about 15% percent of the cortex. The largest part of the cortex is the zona fasciculata comprising 70% of the cortical width. Cells in this zone are responsible for the secretion of glucocorticoid hormones (e.g., corticosterone or cortisol). The innermost portion of the cortex is the zona reticularis (15% of the cortex), which secretes glucocorticoids and in some species small amounts of sex steroids, namely, androgens, estrogens, and progestins. The adrenal cortical cells contain large cytoplasmic lipid droplets consisting of cholesterol and other steroid hormone precursors. Unlike polypeptidehormone-secreting cells, there are no secretory granules in the cytoplasm, since there is direct secretion without significant storage of preformed steroid hormones.

Steroid hormone-producing cells of the adrenal cortex synthesizes a major parent steroid with one to four additional carbon atoms added to the basic 17-carbon steroid nucleus. Since steroid hormones are not stored in any significant amount, a continued rate of synthesis is required to maintain a normal secretory rate. Adrenal steroids are synthesized from cholesterol by specific enzyme-catalyzed reactions confined to a specific cytochrome P450. The common biosynthetic pathway from cholesterol is the formation of pregnenolone, the basic precursor for the three major classes of adrenal steroids in the zona fasciculata. Corticosterone is the major glucocorticoid produced in a manner similar to the production of cortisol. In the zona glomerulosa, pregnenolone is converted to aldosterone In addition to the aforementioned steroid hormones, cells in the zona reticularis also produces small amounts of sex steroids including progesterone, estrogens, and androgens. The mineralocorticoids (e.g., aldosterone) are the major steroids secreted from the zona glomerulosa.

The principal control for the production of glucocorticoids by the zona fasciculata and zona reticularis is exerted by adrenocorticotropin (ACTH) produced in the adenohypophysis of the pituitary gland.

Acute toxicity of the adrenal cortex can have multiple morphologic manifestations as a consequence of impaired steroidogenesis. Chronic toxicity of the adrenal cortex can lead to atrophy, nodular regeneration, fibrosis, or primary proliferation of cortical cells.

There are a variety of spontaneous lesions in the adrenal cortex that must be differentiated from chemically induced lesions considering the mechanisms of toxicity. Acute toxicity of the adrenal cortex can have multiple morphologic manifestations as a consequence of impaired steroid genesis. Chronic toxicity of the adrenal cortex can lead to atrophy, nodular regeneration, fibrosis, or primary proliferation of cortical cells. The reason the adrenal cortex is predisposed to the toxic effects of xenobiotic chemicals appears to be related to at least two factors. First, adrenal cortical cells of most animal species contain large stores of lipids used primarily as substrate for steroid genesis.

Many adrenal cortical toxic compounds are lipophilic and therefore can accumulate in these lipid-rich cells. Second, adrenal cortical cells have enzymes capable of metabolizing of xenobiotic chemicals, including enzymes of the cytochrome P450 family. A number of toxic xenobiotic chemicals serve as pseudo substrates for these enzymes and can be metabolized to reactive toxic compounds. These reactive compounds result in direct toxic effects.

Classes of chemicals known to be toxic for the adrenal cortex include short-chain (three- or four-carbon) aliphatic compounds, lipidosis-inducers, and amphiphilic compounds. These compounds frequently produce necrosis, particularly in the zonae fasciculata and reticularis. Examples include acrylonitrile, 3-aminopropionitrile, 3-bromopropionitrile, 1-butanethiol, and 1,4-butanedithiol. The zonae reticularis and fasciculata appear to be the principal targets of xenobiotic chemicals in the adrenal cortex. Examples of the compounds causing lipidosis include aminoglutethimide, amphenone, and anilines. Tricresyl phosphate (TCP) and other triaryl phosphates cause a defect in cholesterol metabolism. Biologically active cationic amphiphilic compounds produce a generalized phospholipidosis that involves primarily the zonae reticularis and fasciculata and produce microscopic phospholipid rich inclusions. These compounds affect the functional integrity of lysosomes. Another class of compounds that affect the adrenal cortex are hormones, particularly natural and synthetic steroids. The administration of exogenous steroid hormones may cause functional inactivity, trophic atrophy following prolonged use or proliferative lesions in the adrenal cortex. In addition, there is a miscellaneous group of chemicals that affect hydroxylation and other functions of mitochondrial and microsomal fractions in the adrenal cortex. Examples of these compounds include DDD and DMNM. Other compounds cause their effects by means of cytochrome P450 metabolism and the production of toxic metabolites. A classic example is the activation of carbon tetrachloride, resulting in lipid peroxidation and covalent binding to cellular macromolecules of the adrenal cortex. Many of the chemicals that cause morphologic changes in the adrenal glands also affect cortical function. Chemically induced changes in adrenal function result either from blockage of the action of adrenocorticoids at peripheral sites or by inhibition of synthesis and/or secretion of hormone. Xenobiotic chemicals affecting adrenal function often do so by altering steroidogenesis and result in histological and ultrastructural changes in adrenal cortical cells. For example, chemicals causing increased lipid droplets often inhibit the utilization of steroid precursors, including the conversion of cholesterol to pregnenolone. Chemicals that affect the fine structure of mitochondria and smooth endoplasmic reticulum are associated with lesions primarily in the zonae reticularis and fasciculata. Atrophy of the zona glomerulosa may reflect specific inhibition of aldosterone synthesis or secretion, either directly or indirectly.

Due to the diversity of possible actions of agents to affect the adrenal gland cortex, the assessment of disorders of the adrenal gland cortex is a rather complex process. The current methods usually comprise clinical investigations (e.g. ultrasonography), pathological and histopathological investigations as well as a biochemical and hormone analyses.

However, the biomarkers are rather complex regulated and changes may sometimes occur even at rather progressed stages. Major drawbacks of the histopathological assessments are that they are invasive, and even when combined with the clinical pathology measurements or hormone analyses they are less reliable because they are in part based on the individual interpretations of toxicologist carrying out the investigations or the selected methods for hormone measurements (see Capen 2001, Toxiol. Pathol., 29, 8-33; Rosol 2001, Toxicol. Pathol., 29, 41-48; Szabo 1989, Toxicol. Pathol., 17, 317-329; Tucker 1998, The endocrine system, in: Target organ pathology, a basic text, Turton J and Hooson J (eds) Taylor & Francis, London, United Kingdom, 1998).

The importance to investigate disorders of the adrenal gland cortex may become apparent if one considers that the adrenal gland is one of the most common endocrine organs affected by chemically induced lesions. Moreover, chemical compounds which are used in any kind of industry in the European Community, e.g., will now need to comply with REACH (Registration, Evaluation and Authorisation of Chemicals). In other countries, similar toxicological risk assessments need to be done, e.g., the Material Safety Data Sheets (MSDS) in the US. It will be understood that the potential of a chemical compound to induce disorders of the adrenal gland cortex will be deemed as a high risk for the compound and, consequently, the compound will be available only for limited applications and when obeying high security standards.

Aromatase is a cytochrome P450 enzyme complex responsible for estrogen biosynthesis and converts androgens, such as testosterone and androstenedione, into the estrogens estradiol and estrone. Estrogens are sex steroid hormones that are necessary for female reproduction and affect the development of secondary sex characteristics of females. Estrogens are biosynthesized from cholesterol by a series of enzymatic steps, with the last step involving the conversion of androgens into estrogens by the enzyme aromatase. Estrogen biosynthesis occurs primarily in the ovary in mature, premenopausal women. During pregnancy, the placenta is the main source of estrogen biosynthesis and pathways for production change. Small amounts of these hormones are also synthesized by the testes in the male and by the adrenal cortex, the hypothalamus, and the anterior pituitary in both sexes. The major source of estrogens in both postmenopausal women and men occurs in extraglandular sites, particularly in adipose tissue.

Aromatase is present in the ovary, placenta, uterus, testis, brain, and extraglandular adipose tissues. Two proteins, cytochrome P450 and NADPH-cytochrome P450 reductase, are necessary for enzymatic activity, and the enzyme complex is localized in the smooth endoplasmic reticulum. Aromatase is found in breast tissue, and the importance of intratumoral aromatase and local estrogen production is being unraveled.

In addition to the extensive investigations on the importance of human aromatase in normal physiology and in cancer, this enzyme complex has also been well studied in other mammalian species (rodents, cows, pigs, horses). In many species, aromatase expression and/or activity is restricted to the gonads and the brain. Placental aromatase is found only in primates, cattle, horses, and pigs. Aromatase in the adult rat ovary is regulated by the gonadotropins, FSH and LH, and is induced by elevation in cAMP levels and subsequent activation of protein kinase A. Rat testicular tissues also contain aromatase and convert androgens into estrogens locally for spermatogenesis. The importance of aromatase in the brain was first demonstrated in vivo in rodents, and this conversion of androgens to estrogens in the brain is responsible for neural cell differentiation.

The physiologic balance between different sex steroid hormones is crucial for the development, maintenance, and function of the reproductive system as well as for the differentiation of the sexual phenotype during ontogeny. Estrogens (estrone and estradiol) are products of the androgens (androstenedione and testosterone), and the reaction is catalyzed by aromatase. Therefore, disturbances in aromatase expression and/or changes in its catalytic activity are expected to exhibit negative effects on reproduction parameters. In fact, in transgenic male mice overexpressing aromatase in the testis, the serum estradiol level is increased. Half of these animals were infertile and had larger testes and a significantly increased incidence of Leydig cell tumour in testes.

Based on the inhibitory activity of azoles on key enzymes involved in sex steroid hormone synthesis, it is likely that effects on fertility, sexual behaviour, and reproductive organ development will occur depending on dose level and duration of treatment of laboratory animals.

One potential endocrine target for environmental chemicals is the enzyme aromatase, which catalyzes the biosynthesis of estrogens. In addition, effective aromatase inhibitors have been developed as therapeutic agents for estrogen-dependent breast cancer to reduce the growth stimulatory effects of estrogens in breast cancer. Investigations on the development of aromatase inhibitors began in the 1970's and have expanded greatly in the past three decades. Numerous flavonoids and related phytoestrogen derivatives have been extensively evaluated for their ability to inhibit aromatase activity for two primary reasons: these plant natural products can serve as possible leads for the development of new nonsteroidal aromatase inhibitors; and humans and other animals are exposed to these agents through the diet.

Interpretation of aromatase changes in a toxicological setting may be quite complex and may involve both local as well as systemic manifestations of toxicity and/or pharmacologic response. In a general way, aromatase changes can be classified as either quantitative or qualitative. Alterations in the amount of aromatase present or in the catalytic activity of the enzyme will alter the levels of estrogens in tissues and dramatically disrupt estrogen hormone action. Inhibition of aromatase alters the catalytic activity of the enzyme and results in a rapid decrease in the levels of estrogens. This mechanism of enzyme inhibition is the reason for the therapeutic effectiveness of aromatase inhibitors in the treatment of estrogen-dependent breast cancer and illustrates the importance of estrogen levels to estrogen action. Suppression or induction of the aromatase protein levels also can dramatically influence the subsequent levels of estrogens in tissues and effect hormone action. Environmental agents, toxicants, and various natural products can act via aromatase inhibition and/or alteration in aromatase protein levels to result in altered levels of estrogen and function as endocrine disruptors.

Studies have been conducted in mammalian species to evaluate the effect of endocrine disruptors on the reproductive parameters of both males and females. Several pesticides, including fenarimol, imazalil, and triadimefon, have found to inhibit aromatase in vivo. In two-generation studies in rats, fenarimol caused reduced fertility and a reduction in live-born litter size. Parturition in females was also reduced. Reproduction studies in different species showed that fenarimol had adverse effects on reproductive parameters and hormone levels in rats and mice, but not in guinea pigs or rabbits. At high doses, azole fungicides and other azole compounds affect reproductive organs, fertility, and development in several species. Several studies have provided support for the hypothesis that estrogenic actions could be due to increased aromatase activity. For example, the feminizing effect of atrazine on some species has been hypothesized to be caused by its ability to alter aromataseinduced conversion of androgens to estrogens. In man side effects of aromatase inhibitors covers an increase in joint disorders; an increase in the incidence of osteoporosis and fractures, hypercholesterolemia and osteonecrosis, decreased rate of bone maturation and growth, decreased sperm production, infertility, aggressive behaviour, adrenal insufficiency, kidney failure and liver dysfunction.

Due to the diversity of possible actions, the assessment of effects of aromatase inhibition is a rather complex process. The current methods usually comprise hematological investigations, pathological and histopathological investigations as well as a biochemical analysis. However, the biomarkers are rather complex regulated and changes may sometimes occur even at rather progressed stages. Major drawbacks of the histopathological assessments are that they are invasive, and even when combined with the clinical pathology/hematology measurements that they are less reliable because they are in part based an the individual interpretations of toxicologist carrying out the investigations (see Capen 2008, Toxic responses of the endocrine system, Chapter 21, in: Casarett & Doull's Toxicology, The basic science of poisons, Klaassen C D (ed.), McGraw-Hill P, 7th revised edition, New York (2008); US EPA (2005) Final detailed review paper on Aromatase, EPA contract No. 68-W-01-023, prepared by Battelle, Columbus, Ohio for US EPA endocrine disruptor screening program, Washington, US, March 2005; Zarn 2003, Environ. Health. Perspect., 111, 255-261)

Sensitive and specific methods for determining efficiently and reliably aromatase inhibition and, in particular, the early onset thereof are not available but would, nevertheless, be highly appreciated. The importance of aromatase inhibition may become apparent if one considers its consequences on tumor development. Moreover, chemical compounds which are used in any kind of industry in the European Community, e.g., will now need to comply with REACH (Registration, Evaluation and Authorization of Chemicals). In other countries, similar toxicological risk assessments need to be done, e.g., the Material Safety Data Sheets (MSDS) in the US. It will be understood that the potential of a chemical compound to induce aromatase inhibition with its consequences on tumor development will be deemed as a high risk for the compound and, consequently, the compound will be available only for limited applications and when obeying high security standards.

Androgens are a group of hormones that stimulates or controls the development and maintenance of male characteristics by binding to androgen receptors that primarily influence the growth and development of the male reproductive system including the activity of the accessory male sex organs and development of male secondary sex characteristics. Androgens are also the original anabolic steroids and the precursor of all estrogens, the female sex hormones. The predominant and most active androgen is testosterone, which is produced by the interstitial Leydig cells of the testes. The actual secretion of androgens by these cells is controlled by luteinizing hormone (LH) from the pituitary gland. The other androgens, which support the functions of testosterone, are produced mainly by the adrenal cortex and only in relatively small quantities includes any of the 19-carbon steroids synthesized by the adrenal cortex, the inner portion of the adrenal gland, that function as weak steroids or steroid precursors, including dehydroepiandrosterone (DHEA), dehydroepiandrosterone sulfate (DHEA-S), androstenedione, androstenediol, androsterone, and dihydrotestosterone (DHT).

Testosterone is the principal androgen in males, secreted in the response to luteinizing hormone released from the pituitary gland, can directly bind to androgen receptors and is converted in some tissues to dihydrotestosterone, which binds to androgen receptors with higher potency. Activation of androgen receptors results in differentiation of the external genitalia, increased hair growth during puberty, and stimulation of the prostate gland. Testosterone also contributes to the mass and strength of skeletal muscle. Testosterone is also converted to estrogen, which then binds to estrogen receptors and mediates closure of the epiphyses in the bone.

In general, an androgen antagonist (anti-androgen) is any of a group of hormone receptor antagonist compounds that are capable of preventing or inhibiting the biologic effects on normally responsive tissues in the body. Anti-androgens usually work by blocking the appropriate receptors, competing for binding sites on the cell's surface, obstructing the androgens' pathway. Xenobiotic chemicals acting as androgen receptor antagonists can disrupt the hypothalamic-pituitary-testis axis at one of several possible sites interfering with negative feedback control, resulting in hyperfunction of the pituitary gland, such as procymidone due to binding to the androgen receptor, increases circulating levels of LH and results in stimulation of Leydig cells, leading to an increased incidence of hyperplasia and adenomas in rats. Another indicator of reduced plasma testosterone levels is the size of the seminal vesicles and prostate. Their secretory function is androgen dependent and very sensitive to circulating concentrations of testosterone. However, secretory function may also be affected by inhibitors of 5-alpha reductase, which metabolizes testosterone to dihydrotestosterone.

Chemicals that bind to the androgen receptor in an antagonistic fashion include the pharmaceuticals cimetidine, cyproterone acetate, and hydroxyflutamide. Ketoconazole, an antifungal drug, blocks the synthesis of steroids, including testosterone and cortisol. Spironolactone, a diuretic, is also a weak inhibitor of the androgen receptor and a weak inhibitor of testosterone synthesis. Androgen-receptor antagonists can be used in combination with a gonadotropin-releasing hormone (GnRH) analog in the treatment of metastatic prostate cancer. Environmental chemicals that have been shown to act as androgen receptor antagonists include the metabolites of the agricultural fungicide vinclozolin, the DDT metabolite DDE, some hydroxylated PCBs, and the organophosphate insecticide fenitrothion. The consequence of androgen receptor antagonism is typically considered demasculinization and in laboratory animal studies have included reductions in the size of the ventral prostate and seminal vesicle weights along with deformities of the penis.

Chemicals often can function as either a receptor agonist or antagonist depending on the level of endogenous hormone. A weak agonist may bind to a receptor and stimulate some low-level receptor-mediated activity in the absence of the endogenous hormone. However, in the presence of the hormone, binding of the xenobiotic to the receptor may prevent binding of the endogenous hormone, and if the xenobiotic is a much weaker activator of receptor-mediated activity, then the net effect is loss of activity. Thus, in the presence of the endogenous hormone, the xenobiotic functions as a receptor antagonist. Whether a weak chemical-agonist functions as an agonist or antagonist depends on the concentration, the binding affinity to the receptor, the concentration of the endogenous hormone to the receptor, and the binding affinity of the endogenous hormone to the receptor.

In addition, chemicals can also act as receptor agonists and stimulate receptor-dependent physiological processes in the absence of the endogenous receptor hormone. Such inappropriate stimulation can result in the errant expression of hormone-dependent processes such as gynecomastia in males.

Due to the diversity of possible actions of agents to impair the androgen function as agonists or antagonists, the assessment is a rather complex process. The current methods usually comprise pathological and histopathological investigations as well as a biochemical and hormone analysis. However, the biomarkers are rather complex regulated and changes may sometimes occur even at rather progressed stages. Major drawbacks of the histopathological assessments are that they are invasive, and even when combined with the clinical pathology measurements or hormone analysis they are less reliable because they are in part based on the individual interpretations of toxicologist carrying out the investigations or the selected methods for hormone measurements (see Capen 2001, Toxicol. Pathol., 29, 8-33; Capen 2008, Toxic responses of the endocrine system, Chapter 21, in: Casarett & Doull's Toxicology, The basic science of poisons, Klaassen C D (ed.), McGraw-Hill P, 7th revised edition, New York (2008); Capen 1989, Toxicol. Pathol., 17, 234-249; Heindel 1989, Toxicol. Pathol., 17, 411-445; Foster P M D, Gray L E (2008) Toxic responses of the reproductive system, Chapter 20, 761-806, in: Casarett & Doull's Toxicology, The basic science of poisons, Klaassen C D (ed.), McGraw-Hill P, 7th revided edition, New York (2008)).

The importance to investigate impairments of the androgen function may become apparent if one considers that androgens are an integrated part of the endocrine system and as such can be impaired by chemically induced lesions. Moreover, chemical compounds which are used in any kind of industry in the European Community, e.g., will now need to comply with REACH (Registration, Evaluation and Authorisation of Chemicals). In other countries, similar toxicological risk assessments need to be done, e.g., the Material Safety Data Sheets (MSDS) in the US. It will be understood that the potential of a chemical compound to induce disorders of the adrenal gland cortex will be deemed as a high risk for the compound and, consequently, the compound will be available only for limited applications and when obeying high security standards.

Estrogens are a group of hormones that primarily influence the female reproductive tract in its development, maturation, and function. While estrogens are present in both men and women, they are usually present at significantly higher levels in women of reproductive age. There are three major estrogenic hormones, estradiol as the predominant one and estrone and estriol. The major sources of estrogens are the ovaries and the placenta; additional small amounts are secreted by the adrenal glands, the liver, the breast and by the male testes. The follicle and interstitial cells in the ovaries are the predominant production sites of estrogens in the female. Estrogen levels are highest during ovulation and after menstruation, when the corpus luteum replaces the empty follicle. Androgens are converted to estrogens by an aromatase. The ovaries are the richest source of aromatase. Estradiol, the most potent estrogen, is synthesized from testosterone. Estrone can be formed from estradiol, but its major precursor is androstenedione. Estriol, the weakest of the estrogens, is formed from both estrone and estradiol. Estrogens bind reversibly to a protein known as sex hormone-binding globulin and at its target tissues bind to the estrogen receptor. In females, estrogens affect the ovaries, vagina, fallopian tubes, uterus, and mammary glands. Estrogens influence the structural differences between the male and female bodies. In the male, traces of estrogens are present in the blood and urine; estrogens seem to be most evident in the male during puberty and old age. In males, estrogens regulate certain functions of the reproductive system important to the maturation of sperm and are necessary for a healthy libido.

Estrogens coordinate the growth and maintenance of the reproductive tract, pituitary, breasts, and other tissues. Estrogens are also responsible for maturation of the skeleton and development of female secondary sex characteristics when females enter puberty. The other important functions of estrogens include modulation of many metabolic processes, e.g., hepatic metabolism. The production of sex hormone-binding globulin, thyroxine-binding globulin, blood-clotting factors and plasminogen in the liver is stimulated by estrogens. Estrogens stimulate cellular proliferation, induce RNA and protein synthesis of uterine and ovary tissues, and increase the size of the cells. This effect leads to the growth and regeneration of the endometrial layer and increase in the number and size of endometrial glands. Under the influence of estrogen, vaginal mucosa becomes thicker, as cervical mucus becomes thinner.

Chemicals often can function as either a receptor agonist or antagonist depending on the level of endogenous hormone. A weak agonist may bind to a receptor and stimulate some low-level receptor-mediated activity in the absence of the endogenous hormone. However, in the presence of the hormone, binding of the xenobiotic to the receptor may prevent binding of the endogenous hormone, and if the xenobiotic is a much weaker activator of receptor-mediated activity, then the net effect is loss of activity. Thus, in the presence of the endogenous hormone, the xenobiotic functions as a receptor antagonist. Whether a weak chemical-agonist functions as an agonist or antagonist depends on the concentration, the binding affinity to the receptor, the concentration of the endogenous hormone to the receptor, and the binding affinity of the endogenous hormone to the receptor.

For example, the drug tamoxifen functions as an estrogen receptor antagonists in reproductive tissue but functions as an agonist with respect to the preservation of bone mineral density and reducing serum cholesterol concentrations. Accordingly tamoxifen can function as a therapeutic agent against the growth of estrogen-responsive breast cancers and osteoporosis via antagonism and agonism, respectively. Other drugs that bind to the estrogen receptor as an antagonist or mixed agonist/antagonist include raloxifene. Environmental estrogen receptor antagonists include some phytochemicals and PCBs. Consequences of estrogen receptor antagonism are typically considered de-feminization. In laboratory animal studies, estrogen receptor antagonists have been shown in females to disrupt estrous cycles, impair fertility, increase preimplantation loss, and cause embryolethality. Gynecomastia is a common side effect of estrogenic drugs such as DES and fosfestrol when administered to adult males. The physiological consequences of chemical-estrogenic activity is typically characteristic of feminization, that is, the acquisition of female characteristics.

Among the steroid hormone receptors, the estrogen receptor appears most susceptible to the agonistic action of xenobiotics. Estrogen receptor agonists are quite diverse in molecular structure but it is not clear why the estrogen receptor would be more susceptible to the agonistic action of xenobiotics as compared to other steroid hormone receptors. The estrogen receptor is often referred to as a promiscuous receptor because of this susceptibility to agonistic interactions with xenobiotics.

Due to the diversity of possible actions of agents to impair the estrogen function as agonists or antagonists, the assessment is a rather complex process. The current methods usually comprise pathological and histopathological investigations as well as a biochemical and hormone analysis. However, the biomarkers are rather complex regulated and changes may sometimes occur even at rather progressed stages. Major drawbacks of the histopathological assessments are that they are invasive, and even when combined with the clinical pathology measurements or hormone analysis they are less reliable because they are in part based on the individual interpretations of toxicologist carrying out the investigations or the selected methods for hormone measurements (see Capen 2001, Toxicol. Pathol., 29, 8-33; Capen 2008, Toxic responses of the endocrine system, Chapter 21, in: Casarett & Doull's Toxicology, The basic science of poisons, Klaassen C D (ed.), McGraw-Hill P, 7th revised edition, New York (2008); Capen 1989, Toxicol. Pathol., 17, 234-249; Foster 2008, Toxic responses of the reproductive system, Chapter 20, 761-806, in: Casarett & Doull's Toxicology, The basic science of poisons, Klaassen C D (ed.), McGraw-Hill P, 7th revised edition, New York (2008); Guo J Z, Hahn D W, Wachter M P, Johnson R W (2000) Hormones, estrogens and antiestrogens, 1-32, in: Kirk-Othmer, Encyclopedia of Chemical Toxicology, John Wiley & Sons, Inc. http://www.scribd.com/doc/30137262/Hormones-Estrogens-and-Antiestrogens).

The importance to investigate impairments of the estrogen function may become apparent if one considers that estrogens are an integrated part of the endocrine system and as such can be impaired by chemically induced lesions. Moreover, chemical compounds which are used in any kind of industry in the European Community, e.g., will now need to comply with REACH (Registration, Evaluation and Authorisation of Chemicals). In other countries, similar toxicological risk assessments need to be done, e.g., the Material Safety Data Sheets (MSDS) in the US. It will be understood that the potential of a chemical compound to induce disorders of the adrenal gland cortex will be deemed as a high risk for the compound and, consequently, the compound will be available only for limited applications and when obeying high security standards.

The pancreas is a retroperitoneal mixed exocrine-endocrine gland in the upper abdomen, which is located between the loop of the duodenum and the spleen. Most of the pancreatic tissue is devoted to exocrine function, in which digestive enzymes are secreted via the pancreatic ducts into the duodenum. The cells in the pancreas that produce digestive enzymes are called acinar cells. The endocrine pancreas consists of the islets of Langerhans. There are approximately one million islets that are scattered throughout the pancreas. Approximately 75 percent of the cells in each islet are insulin-producing beta cells, which are clustered centrally in the islet. The alpha cells of the islets of Langerhans produce an opposing hormone, glucagon, which releases glucose from the liver and fatty acids from fat tissue. In turn, glucose and free fatty acids favor insulin release and inhibit glucagon release. The delta cells produce somatostatin, a strong inhibitor of somatotropin, insulin, and glucagon; its role in metabolic regulation is not yet clear. The remainder of each islet consists of F (or PP) cells, which secrete pancreatic polypeptide, respectively, and are located at the periphery of the islet. Each islet is supplied by one or two very small arteries that branch into numerous capillaries. The islets also contain many nerve endings.

The pancreas receives regulatory innervation via hormones in the blood and through the autonomic nervous system. These two inputs regulate the secretory activity of the pancreas. Sympathetic (adrenergic) a2 decreases secretion from beta cells, increases and secretion from alpha cells while parasympathetic (muscarinic) M3 increases stimulation of alpha cells and beta cells.

The principal function of the endocrine pancreas is the secretion of insulin and other polypeptide hormones necessary for the cellular storage or mobilization of glucose, amino acids, and triglycerides. Islet function may be regulated by signals initiated by autonomic nerves, circulating metabolites, circulating hormones, or local hormones.

The importance of the endocrine pancreas is that insulin plays a central role in the regulation of energy metabolism. A relative or absolute deficiency of insulin leads to diabetes mellitus. Insulin stimulates the transport of glucose into tissues, the transport of amino acids into tissues, and the transport of fatty acids into tissues. In the fasting state, insulin secretion decreases and glucagon secretion increases. Liver glycogen stores, followed later by protein and fat stores, are mobilized to produce glucose. Ultimately, most nutrient needs are provided by fatty acids mobilized from fat stores. The pancreatic hormone glucagon also plays a key role in maintaining glucose homeostasis and in regulating nutrient storage. An adequate supply of glucose is required for optimal body growth and development and for the function of the central nervous system, for which glucose is the major source of energy.

The pancreas may be the site of acute and chronic infections, tumors, and cysts. Numerous chemical toxicants have been identified for the endocrine component. These include ethanol, alloxan, azaserine. dimethylbenzo[a]anthracene, ethionine, 4-hydroxyaminoquinoline-1-oxide, beta-oxidized derivatives of dipropylnitrosamine. oleic acid, and streptozotocin. Depending on the toxicant, the cellular effects span from acute injury and death, to hyperplasia, metaplasia and malignant transformation. In addition, therapeutic drugs are toxic for the pancreas and comprised of azathioprine, estrogens, furosemide, methyldopa, pentamidine, procainamide, sulfonamides, and thiazide diuretics. From the above it is evident that the endocrine pancreas can be influenced and impaired at different levels and by different stimuli. Besides genetic influences, exogenous stimuli such as xenobiotic chemicals may impair endocrine pancreas function, especially with regards to hormone homeostasis.

Due to the diversity of possible actions of agents to affect the endocrine pancreas, the assessment of disorders is a rather complex process. The current methods usually comprise clinical investigations (e.g. ultrasonography), pathological and histopathological investigations as well as a biochemical and hormone analyses.

However, the biomarkers are rather complex regulated and changes may sometimes occur even at rather progressed stages. Major drawbacks of the histopathological assessments are that they are invasive, and even when combined with the clinical pathology measurements or hormone analyses they are less reliable because they are in part based on the individual interpretations of toxicologist carrying out the investigations or the selected methods for hormone measurements (see Scarpelli 1989, Toxicol. Appl. Pharmacol., 101, 543-554; Turton 1998, (eds) Target organ pathology, a basic text, Taylor & Francis, London, United Kingdom, 1998; Utiger 2010, The pancreas, Anatomy and exocrine and endocrine functions in Encyclopedia Britannica. Retrieved Oct. 26, 2010, from Encyclopedia Britannica Online: http://www.britannica.com/EBchecked/topic/440971/pancreas).

Sensitive and specific methods for determining efficiently and reliably endocrine pancreas disorders and, in particular, the early onset thereof are not available but would, nevertheless, be highly appreciated. Moreover, chemical compounds which are used in any kind of industry in the European Community, e.g., will now need to comply with REACH (Registration, Evaluation and Authorisation of Chemicals). In other countries, similar toxicological risk assessments need to be done, e.g., the Material Safety Data Sheets (MSDS) in the US. It will be understood that the potential of a chemical compound to induce disorders of the adrenal gland cortex will be deemed as a high risk for the compound and, consequently, the compound will be available only for limited applications and when obeying high security standards.

Sensitive and specific methods for assessing the toxicological properties of a chemical compound and, in particular, with respect to their capability of inducing an endocrine disease or disorder, in an efficient and reliable manner are not yet available but would, nevertheless, be highly appreciated.

Thus, the technical problem underlying the present invention could be seen as the provision of means and methods for complying with the aforementioned needs. The technical problem is solved by the embodiments characterized in the claims and described herein below.

Accordingly, the present invention relates to a method for diagnosing an endocrine disease or disorder comprising:
(a) determining the amount of at least one biomarker selected from any one of Tables 1a, 1 b, 2a, 2b, 3a, 3b, 4a, 4b, 4c, 4d, 5a, 5b, 6a, 6b, 7, 8a, 8b, 9, 10a, 10b, 11a, 11 b, 12a, 12b, 12c, 12d, 13a, 13b, 14a, 14b, 15a, 15b, 15c, 15d, 16, 17a, or 17b in a test sample of a subject suspected to suffer from an endocrine disease or disorder, and
(b) comparing the amounts determined in step (a) to a reference, whereby an endocrine disease or disorder is to be diagnosed.

In a particular embodiment of the method of the invention, a method is provided for diagnosing endocrine disease or disorder comprising:
(a) selecting a male or female subject suspected to suffer from endocrine disease or disorder;
(b) obtaining a test sample from said selected subject;
(c) pre-treating said sample in preparation for analysis;
(d) determining the amount of at least one biomarker selected from any one of Tables 1a, 1 b, 2a, 2b, 3a, 3b, 4a, 4b, 4c, 4d, 5a, 5b, 6a, 6b, 7, 8a, 8b, 9, 10a, 10b, 11a, 11 b, 12a, 12b, 12c, 12d, 13a, 13b, 14a, 14b, 15a, 15b, 15c, 15d, 16, 17a, or 17b in said test sample, and
(e) comparing the amounts determined in step (d) to a reference; and
(f) based on the comparison of step (e), diagnose endocrine disease or disorder by monitoring, confirmation or classification of the endocrine disease or disorder or its symptoms.

In a preferred embodiment of the aforementioned method said subject has been brought into contact with a compound suspected to be capable of inducing an endocrine disease or disorder.

The present invention also relates to a method of determining whether a compound is capable of inducing an endocrine disease or disorder in a subject comprising:
(a) determining in a sample of a subject which has been brought into contact with a compound suspected to be capable of inducing an endocrine disease or disorder the amount of at least one biomarker selected from any one of Tables 1a, 1b, 2a, 2b, 3a, 3b, 4a, 4b, 4c, 4d, 5a, 5b, 6a, 6b, 7, 8a, 8b, 9, 10a, 10b, 11a, 11 b, 12a, 12b, 12c, 12d, 13a, 13b, 14a, 14b, 15a, 15b, 15c, 15d, 16, 17a, or 17b; and
(b) comparing the amounts determined in step (a) to a reference, whereby the capability of the compound to induce an endocrine disease or disorder is determined.

In a particular embodiment of the method of the invention, a method is provided for determining whether a compound is capable of inducing endocrine disease or disorder in a subject comprising:
(a1) (i) selecting a male or female subject;
   (ii) bringing said subject into contact with a compound suspected to be capable of inducing endocrine disease or disorder, or
(a2) selecting a male or female subject brought into contact with a compound capable of inducing endocrine disease or disorder;
(b) obtaining a test sample from said selected subject;
(c) pre-treating said sample in preparation for analysis;
(d) determining the amount of at least one biomarker selected from any one of Tables 1a, 1 b, 2a, 2b, 3a, 3b, 4a, 4b, 4c, 4d, 5a, 5b, 6a, 6b, 7, 8a, 8b, 9, 10a, 10b, 11a, 11 b, 12a, 12b, 12c, 12d, 13a, 13b, 14a, 14b, 15a, 15b, 15c, 15d, 16, 17a, or 17b in said test sample, and
(e) comparing the amounts determined in step (d) to a reference; and
(f) based on the comparison of step (e), identifying whether the compound is capable of inducing endocrine disease or disorder, or not.

In a preferred embodiment of the aforementioned method said compound is at least one compound selected from the group consisting of: 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, and Ziprasidone hydrochloride.

In another preferred embodiment of the methods of the present invention said reference is derived from (i) a subject or group of subjects which suffers from an endocrine disease or disorder or (ii) a subject or group of subjects which has been brought into contact with at least one compound selected from the group consisting of: 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, and Ziprasidone hydrochloride. In a more preferred embodinvent of said method essentially identical amounts for the biomarkers in the test sample and the reference are indicative for an endocrine disease or disorder.

In another preferred embodiment of the methods of the present invention said reference is derived from (i) a subject or group of subjects known to not suffer from an endocrine disease or disorder or (ii) a subject or group of subjects which has not been brought into contact with at least one compound selected from the group consisting of: 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, and Ziprasidone hydrochloride. In a more preferred embodiment of said methods amounts for the biomarkers which differ in the test sample in comparison to the reference are indicative for an endocrine disease or disorder.

In yet another embodiment of the methods of the present invention said reference is a calculated reference for the biomarkers for a population of subjects. In a more preferred embodiment of said methods amounts for the biomarkers which differ in the test sample in comparison to the reference are indicative for an endocrine disease or disorder.

The present invention also contemplates a method of identifying a substance for treating an endocrine disease or disorder comprising the steps of:
(a) determining in a sample of a subject suffering from an endocrine disease or disorder which has been brought into contact with a candidate substance suspected to be capable of treating an endocrine disease or disorder the amount of at least one biomarker selected from any one of Tables 1a, 1 b, 2a, 2b, 3a, 3b, 4a, 4b, 4c, 4d, 5a, 5b, 6a, 6b, 7, 8a, 8b, 9, 10a, 10b, 11a, 11 b, 12a, 12b, 12c, 12d, 13a, 13b, 14a, 14b, 15a, 15b, 15c, 15d, 16, 17a, or 17b; and
(b) comparing the amounts determined in step (a) to a reference, whereby a substance capable of treating an endocrine disease or disorder is to be identified.

In a particular embodiment of the method of the invention, a method is provided for identifying a substance for treating endocrine disease or disorder comprising:
(a1) (i) selecting a male or female subject;
   (ii) bringing said subject into contact with a compound suspected to be capable of inducing endocrine disease or disorder such that endocrine disease or disorder is elicited, or
(a2) selecting a male or female suffering from endocrine disease or disorder;
(b) obtaining a test sample from said selected subject;
(c) pre-treating said sample in preparation for analysis;
(d) determining the amount of at least one biomarker selected from any one of Tables 1a, 1 b, 2a, 2b, 3a, 3b, 4a, 4b, 4c, 4d, 5a, 5b, 6a, 6b, 7, 8a, 8b, 9, 10a, 10b, 11a, 11 b, 12a, 12b, 12c, 12d, 13a, 13b, 14a, 14b, 15a, 15b, 15c, 15d, 16, 17a, or 17b in said test sample, and
(e) comparing the amounts determined in step (d) to a reference; and
(f) based on the comparison of step (e), identifying and selecting the substance for treating endocrine disease or disorder.

In a preferred embodiment of the aforementioned method said reference is derived from (i) a subject or group of subjects which suffers from an endocrine disease or disorder or (ii) a subject or group of subjects which has been brought into contact with at least one compound selected from the group consisting of: 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, and Ziprasidone hydrochloride. In a more preferred embodiment of said method amounts for the biomarkers which differ in the test sample and the reference are indicative for a substance capable of treating an endocrine disease or disorder.

In another preferred embodiment of the aforementioned method said reference is derived from (i) a subject or group of subjects known to not suffer from an endocrine disease or disorder or (ii) a subject or group of subjects which has not been brought into contact with at least one compound selected from the group consisting of: 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, and Ziprasidone hydrochloride. In a more preferred embodiment of the said methods essentially identical amounts for the biomarkers in the test sample and the reference are indicative for a substance capable of treating an endocrine disease or disorder.

In yet another preferred embodiment of the aforementioned method said reference is a calculated reference for the biomarkers in a population of subjects. In a more preferred embodiment of the said methods essentially identical amounts for the biomarkers in the test sample and the reference are indicative for a substance capable of treating an endocrine disease or disorder.

The present invention also relates to the use of at least one biomarker selected from any one of Tables 1a, 1 b, 2a, 2b, 3a, 3b, 4a, 4b, 4c, 4d, 5a, 5b, 6a, 6b, 7, 8a, 8b, 9, 10a, 10b, 11a, 11 b, 12a, 12b, 12c, 12d, 13a, 13b, 14a, 14b, 15a, 15b, 15c, 15d, 16, 17a, or 17b or a detection agent for the said biomarker for diagnosing an endocrine disease or disorder in a sample of a subject.

Moreover, the present invention relates to a device for diagnosing an endocrine disease or disorder in a sample of a subject suspected to suffer therefrom comprising:
(a) an analyzing unit comprising a detection agent for at least one biomarker selected from any one of Tables 1a, 1 b, 2a, 2b, 3a, 3b, 4a, 4b, 4c, 4d, 5a, 5b, 6a, 6b, 7, 8a, 8b, 9, 10a, 10b, 11a, 11b, 12a, 12b, 12c, 12d, 13a, 13b, 14a, 14b, 15a, 15b, 15c, 15d, 16, 17a, or 17b which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby an endocrine disease or disorder is diagnosed.

In a preferred embodiment of the device of the invention said stored reference is a reference derived from a subject or a group of subjects known to suffer from an endocrine disease or disorder or a subject or group of subjects which has been brought into contact with at least one compound selected from the group consisting of 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, and Ziprasidone hydrochloride, and said data processor executes instructions for comparing the amount of the at least one biomarker determined by the analyzing unit to the stored reference, wherein an essentially identical amount of the at least one biomarker in the test sample in comparison to the reference is indicative for the presence of an endocrine disease or disorder or wherein an amount of the at least one biomarker in the test sample which differs in comparison to the reference is indicative for the absence of an endocrine disease or disorder.

In another preferred embodiment of the device of the invention said stored reference is a reference derived from a subject or a group of subjects known to not suffer from an endocrine disease or disorder or a subject or group of subjects which has not been brought into contact with at least one compound selected from the group consisting of 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, and Ziprasidone hydrochloride, and said data processor executes instructions for comparing the amount of the at least one biomarker determined by the analyzing unit to the stored reference, wherein an amount of the at least one biomarker in the test sample which differs in comparison to the reference is indicative for the presence of an endocrine disease or disorder or wherein an essential identical amount of the at least one biomarker in the test sample in comparison to the reference is indicative for the absence of an endocrine disease or disorder.

Further, the present invention relates to a kit for diagnosing an endocrine disease or disorder comprising a detection agent for the at least one biomarker selected from any one of Tables 1a, 1b, 2a, 2b, 3a, 3b, 4a, 4b, 4c, 4d, 5a, 5b, 6a, 6b, 7, 8a, 8b, 9, 10a, 10b, 11a, 11 b, 12a, 12b, 12c, 12d, 13a, 13b, 14a, 14b, 15a, 15b, 15c, 15d, 16, 17a, or 17b and standards for the at least one biomarker the concentration of which is derived from a subject or a group of subjects known to suffer from an endocrine disease or disorder or derived from a subject or a group of subjects known to not suffer from an endocrine disease or disorder.

In particular, the present invention relates to a method for diagnosing toxicity of the adrenal cortex comprising:
(a) determining the amount of at least one biomarker selected from any one of Tables 1a, 1b, 2a, 2b, 3a, 3b, 4a, 4b, 4c, 4d, 5a, 5b, 6a or 6b in a test sample of a subject suspected to suffer from toxicity of the adrenal cortex, and
(b) comparing the amounts determined in step (a) to a reference, whereby bone marrow toxicity is to be diagnosed.

In a particular embodiment of the method of the invention, a method is provided for diagnosing toxicity of the adrenal cortex comprising:
(a) selecting a male or female subject suspected to suffer from toxicity of the adrenal cortex;
(b) obtaining a test sample from said selected subject;
(c) pre-treating said sample in preparation for analysis;
(d) determining the amount of at least one biomarker selected from any one of Tables 1a, 1b, 2a, 2b, 3a, 3b, 4a, 4b, 4c, 4d, 5a, 5b, 6a or 6b in said test sample, and
(e) comparing the amounts determined in step (d) to a reference; and
(f) based on the comparison of step (e), diagnose toxicity of the adrenal cortex by monitoring, confirmation or classification of the toxicity of the adrenal cortex or its symptoms.

In a preferred embodiment of the aforementioned method said subject has been brought into contact with a compound suspected to be capable of inducing toxicity of the adrenal cortex.

The present invention also relates to a method of determining whether a compound is capable of inducing toxicity of the adrenal cortex in a subject comprising:
(a) determining in a sample of a subject which has been brought into contact with a compound suspected to be capable of inducing toxicity of the adrenal cortex the amount of at least one biomarker selected from any one of Tables 1a, 1b, 2a, 2b, 3a, 3b, 4a, 4b, 4c, 4d, 5a, 5b, 6a or 6b; and
(b) comparing the amounts determined in step (a) to a reference, whereby the capability of the compound to induce bone marrow toxicity is determined.

In a particular embodiment of the method of the invention, a method is provided for determining whether a compound is capable of inducing toxicity of the adrenal cortex in a subject comprising:
(a1) (i) selecting a male or female subject;
   (ii) bringing said subject into contact with a compound suspected to be capable of inducing toxicity of the adrenal cortex, or
(a2) selecting a male or female subject brought into contact with a compound capable of inducing toxicity of the adrenal cortex;
(b) obtaining a test sample from said selected subject;
(c) pre-treating said sample in preparation for analysis;
(d) determining the amount of at least one biomarker selected from any one of Tables 1a, 1b, 2a, 2b, 3a, 3b, 4a, 4b, 4c, 4d, 5a, 5b, 6a or 6b, in said test sample, and
(e) comparing the amounts determined in step (d) to a reference; and
(f) based on the comparison of step (e), identifying whether the compound is capable of inducing toxicity of the adrenal cortex, or not.

In a preferred embodiment of the aforementioned method said compound is at least one compound selected from the group consisting of: ACTH, Cabergoline, Cyproteron Acetate, Dexamethasone, Epoxiconazole, Fenarimol, Formestane, Methimazole, Mifepristone, Risperidone, Triticonazole, and Vinclozolin.

In another preferred embodiment of the methods of the present invention said reference is derived from (i) a subject or group of subjects which suffers from toxicity of the adrenal cortex or (ii) a subject or group of subjects which has been brought into contact with at least one compound selected from the group consisting of: ACTH, Cabergoline, Cyproteron Acetate, Dexamethasone, Epoxiconazole, Fenarimol, Formestane, Methimazole, Mifepristone, Risperidone, Triticonazole, and Vinclozolin. In a more preferred embodiment of said method essentially identical amounts for the biomarkers in the test sample and the reference are indicative for toxicity of the adrenal cortex.

In another preferred embodiment of the methods of the present invention said reference is derived from (i) a subject or group of subjects known to not suffer from toxicity of the adrenal cortex or (ii) a subject or group of subjects which has not been brought into contact with at least one compound selected from the group consisting of: ACTH, Cabergoline, Cyproteron Acetate, Dexamethasone, Epoxiconazole, Fenarimol, Formestane, Methimazole, Mifepristone, Risperidone, Triticonazole, and Vinclozolin. In a more preferred embodiment of said methods amounts for the biomarkers which differ in the test sample in comparison to the reference are indicative for toxicity of the adrenal cortex.

In yet another embodiment of the methods of the present invention said reference is a calculated reference for the biomarkers for a population of subjects. In a more preferred embodiment of said methods amounts for the biomarkers which differ in the test sample in comparison to the reference are indicative for toxicity of the adrenal cortex.

The present invention also contemplates a method of identifying a substance for treating toxicity of the adrenal cortex comprising the steps of:
(a) determining in a sample of a subject suffering from toxicity of the adrenal cortex which has been brought into contact with a candidate substance suspected to be capable of treating toxicity of the adrenal cortex the amount of at least one biomarker selected from any one of Tables 1a, 1b, 2a, 2b, 3a, 3b, 4a, 4b, 4c, 4d, 5a, 5b, 6a or 6b; and
(b) comparing the amounts determined in step (a) to a reference, whereby a substance capable of treating toxicity of the adrenal cortex is to be identified.

In a particular embodiment of the method of the invention, a method is provided for identifying a substance for treating toxicity of the adrenal cortex comprising:
(a1) (i) selecting a male or female subject;
   (ii) bringing said subject into contact with a compound suspected to be capable of inducing toxicity of the adrenal cortex such that toxicity of the adrenal cortex is elicited, or
(a2) selecting a male or female suffering from toxicity of the adrenal cortex;
(b) obtaining a test sample from said selected subject;
(c) pre-treating said sample in preparation for analysis;
(d) determining the amount of at least one biomarker selected from any one of Tables 1a, 1b, 2a, 2b, 3a, 3b, 4a, 4b, 4c, 4d, 5a, 5b, 6a or 6b in said test sample, and
(e) comparing the amounts determined in step (d) to a reference; and (f) based on the comparison of step (e), identifying and selecting the substance for treating toxicity of the adrenal cortex.

In a preferred embodiment of the aforementioned method said reference is derived from (i) a subject or group of subjects which suffers from toxicity of the adrenal cortex or (ii) a subject or group of subjects which has been brought into contact with at least one compound selected from the group consisting of: ACTH, Cabergoline, Cyproteron Acetate, Dexamethasone, Epoxiconazole, Fenarimol, Formestane, Methimazole, Mifepristone, Risperidone, Triticonazole, and Vinclozolin. In a more preferred embodiment of said method amounts for the biomarkers which differ in the test sample and the reference are indicative for a substance capable of treating toxicity of the adrenal cortex.

In another preferred embodiment of the aforementioned method said reference is derived from (i) a subject or group of subjects known to not suffer from toxicity of the adrenal cortex or (ii) a subject or group of subjects which has not been brought into contact with at least one compound selected from the group consisting of: ACTH, Cabergoline, Cyproteron Acetate, Dexamethasone, Epoxiconazole, Fenarimol, Formestane, Methimazole, Mifepristone, Risperidone, Triticonazole, and Vinclozolin. In a more preferred embodiment of the said methods essentially identical amounts for the biomarkers in the test sample and the reference are indicative for a substance capable of treating toxicity of the adrenal cortex.

In yet another preferred embodiment of the aforementioned method said reference is a calculated reference for the biomarkers in a population of subjects. In a more preferred embodiment of the said methods essentially identical amounts for the biomarkers in the test sample and the reference are indicative for a substance capable of treating toxicity of the adrenal cortex.

The present invention also relates to the use of at least one biomarker selected from any one of Tables 1a, 1b, 2a, 2b, 3a, 3b, 4a, 4b, 4c, 4d, 5a, 5b, 6a or 6b or a detection agent for the said biomarker for diagnosing toxicity of the adrenal cortex in a sample of a subject.

Moreover, the present invention relates to a device for diagnosing toxicity of the adrenal cortex in a sample of a subject suspected to suffer therefrom comprising:
(a) an analyzing unit comprising a detection agent for at least one biomarker selected from any one of Tables 1a, 1 b, 2a, 2b, 3a, 3b, 4a, 4b, 4c, 4d, 5a, 5b, 6a or 6b which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby toxicity of the adrenal cortex is diagnosed.

In a preferred embodiment of the device of the invention said stored reference is a reference derived from a subject or a group of subjects known to suffer from toxicity of the adrenal cortex or a subject or group of subjects which has been brought into contact with at least one compound selected from the group consisting of ACTH, Cabergoline, Cyproteron Acetate, Dexamethasone, Epoxiconazole, Fenarimol, Formestane, Methimazole, Mifepristone, Risperidone, Triticonazole, and Vinclozolin, and said data processor executes instructions for comparing the amount of the at least one biomarker determined by the analyzing unit to the stored reference, wherein an essentially identical amount of the at least one biomarker in the test sample in comparison to the reference is indicative for the presence of toxicity of the adrenal cortex or wherein an amount of the at least one biomarker in the test sample which differs in comparison to the reference is indicative for the absence of toxicity of the adrenal cortex.

In another preferred embodiment of the device of the invention said stored reference is a reference derived from a subject or a group of subjects known to not suffer from toxicity of the adrenal cortex or a subject or group of subjects which has not been brought into contact with at least one compound selected from the group consisting of ACTH, Cabergoline, Cyproteron Acetate, Dexamethasone, Epoxiconazole, Fenarimol, Formestane, Methimazole, Mifepristone, Risperidone, Triticonazole, and Vinclozolin, and said data processor executes instructions for comparing the amount of the at least one biomarker determined by the analyzing unit to the stored reference, wherein an amount of the at least one biomarker in the test sample which differs in comparison to the reference is indicative for the presence of toxicity of the adrenal cortex or wherein an essential identical amount of the at least one biomarker in the test sample in comparison to the reference is indicative for the absence of toxicity of the adrenal cortex.

Further, the present invention relates to a kit for diagnosing toxicity of the adrenal cortex comprising a detection agent for the at least one biomarker selected from any one of Tables 1a, 1b, 2a, 2b, 3a, 3b, 4a, 4b, 4c, 4d, 5a, 5b, 6a or 6b and standards for the at least one biomarker the concentration of which is derived from a subject or a group of subjects known to suffer from toxicity of the adrenal cortex or derived from a subject or a group of subjects known to not suffer from toxicity of the adrenal cortex.

In particular, the present invention relates to a method for diagnosing a disease or disorder related to improper sex hormone homeostasis comprising:
(a) determining the amount of at least one biomarker selected from any one of Tables 7, 8a, 8b, 9, 10a, 10b, 11a, 11b, 12a, 12b, 12c, 12d, 13a, 13b, 14a, 14b, 15a, 15b, 15c, 15d, or 16 in a test sample of a subject suspected to suffer from a disease or disorder related to improper sex hormone homeostasis, and
(b) comparing the amounts determined in step (a) to a reference, whereby a disease or disorder related to improper sex hormone homeostasis is to be diagnosed.

In a particular embodiment of the method of the invention, a method is provided for diagnosing a disease or disorder related to improper sex hormone homeostasis comprising:
(a) selecting a male or female subject suspected to suffer from a disease or disorder related to improper sex hormone homeostasis;
(b) obtaining a test sample from said selected subject;
(c) pre-treating said sample in preparation for analysis;
(d) determining the amount of at least one biomarker selected from any one of Tables 7, 8a, 8b, 9, 10a, 10b, 11a, 11b, 12a, 12b, 12c, 12d, 13a, 13b, 14a, 14b, 15a, 15b, 15c, 15d or 16, in said test sample, and
(e) comparing the amounts determined in step (d) to a reference; and
(f) based on the comparison of step (e), diagnose a disease or disorder related to improper sex hormone homeostasis by monitoring, confirmation or classification of the a disease or disorder related to improper sex hormone homeostasis or its symptoms.

In a preferred embodiment of the aforementioned method said subject has been brought into contact with a compound suspected to be capable of inducing a disease or disorder related to improper sex hormone homeostasis.

The present invention also relates to a method of determining whether a compound is capable of inducing a disease or disorder related to improper sex hormone homeostasis in a subject comprising:
(a) determining in a sample of a subject which has been brought into contact with a compound suspected to be capable of inducing a disease or disorder related to improper sex hormone homeostasis the amount of at least one biomarker selected from any one of Tables 7, 8a, 8b, 9, 10a, 10b, 11a, 11 b, 12a, 12b, 12c, 12d, 13a, 13b, 14a, 14b, 15a, 15b, 15c, 15d, or 16; and
(b) comparing the amounts determined in step (a) to a reference, whereby the capability of the compound to induce a disease or disorder related to improper sex hormone homeostasis is determined.

In a particular embodiment of the method of the invention, a method is provided for determining whether a compound is capable of inducing a disease or disorder related to improper sex hormone homeostasis in a subject comprising:
(a1) (i) selecting a male or female subject;
   (ii) bringing said subject into contact with a compound suspected to be capable of inducing a disease or disorder related to improper sex hormone homeostasis, or
(a2) selecting a male or female subject brought into contact with a compound capable of inducing a disease or disorder related to improper sex hormone homeostasis;
(b) obtaining a test sample from said selected subject;
(c) pre-treating said sample in preparation for analysis;
(d) determining the amount of at least one biomarker selected from any one of Tables 7, 8a, 8b, 9, 10a, 10b, 11a, 11 b, 12a, 12b, 12c, 12d, 13a, 13b, 14a, 14b, 15a, 15b, 15c, 15d or 16 in said test sample, and
(e) comparing the amounts determined in step (d) to a reference; and
(f) based on the comparison of step (e), identifying whether the compound is capable of inducing a disease or disorder related to improper sex hormone homeostasis, or not.

In a preferred embodiment of the aforementioned method said compound is at least one compound selected from the group consisting of: 17-alpha-Ethynylestradiol, 17-alpha-Methyltestosterone, Bromocriptine Mesylate, Buserelin acetate s.c., Cabergoline, Cyproteron Acetate, Diethanolamine, Diethylstilboestrol dipropionate, Fluoxetine hydrochloride, Flutamide, Genistein, Ifosfamide, Iprodione, Leuprolide acetate salt, Medroxyprogesterone acetat, Metazachlor, Methimazole, Mibolerone, Naphthylisothiocyanate, Norethindrone acetate, Raloxifene Hydrochloride, Tamoxifen, Trenbolone, Triticonazole, and Vinclozolin.

In another preferred embodiment of the methods of the present invention said reference is derived from (i) a subject or group of subjects which suffers from a disease or disorder related to improper sex hormone homeostasis or (ii) a subject or group of subjects which has been brought into contact with at least one compound selected from the group consisting of: 17-alpha-Ethynylestradiol, 17-alpha-Methyltestosterone, Bromocriptine Mesylate, Buserelin acetate s.c., Cabergoline, Cyproteron Acetate, Diethanolamine, Diethylstilboestrol dipropionate, Fluoxetine hydrochloride, Flutamide, Genistein, Ifosfamide, Iprodione, Leuprolide acetate salt, Medroxyprogesterone acetat, Metazachlor, Methimazole, Mibolerone, Naphthylisothiocyanate, Norethindrone acetate, Raloxifene Hydrochloride, Tamoxifen, Trenbolone, Triticonazole, and Vinclozolin. In a more preferred embodiment of said method essentially identical amounts for the biomarkers in the test sample and the reference are indicative for a disease or disorder related to improper sex hormone homeostasis.

In another preferred embodiment of the methods of the present invention said reference is derived from (i) a subject or group of subjects known to not suffer from a disease or disorder related to improper sex hormone homeostasis or (ii) a subject or group of subjects which has not been brought into contact with at least one compound selected from the group consisting of: 17-alpha-Ethynylestradiol, 17-alpha-Methyltestosterone, Bromocriptine Mesylate, Buserelin acetate s.c., Cabergoline, Cyproteron Acetate, Diethanolamine, Diethylstilboestrol dipropionate, Fluoxetine hydrochloride, Flutamide, Genistein, Ifosfamide, Iprodione, Leuprolide acetate salt, Medroxyprogesterone acetat, Metazachlor, Methimazole, Mibolerone, Naphthylisothiocyanate, Norethindrone acetate, Raloxifene Hydrochloride, Tamoxifen, Trenbolone, Triticonazole, and Vinclozolin. In a more preferred embodiment of said methods amounts for the biomarkers which differ in the test sample in comparison to the reference are indicative for a disease or disorder related to improper sex hormone homeostasis.

In yet another embodiment of the methods of the present invention said reference is a calculated reference for the biomarkers for a population of subjects. In a more preferred embodiment of said methods amounts for the biomarkers which differ in the test sample in comparison to the reference are indicative for a disease or disorder related to improper sex hormone homeostasis.

The present invention also contemplates a method of identifying a substance for treating a disease or disorder related to improper sex hormone homeostasis comprising the steps of:
(a) determining in a sample of a subject suffering from a disease or disorder related to improper sex hormone homeostasis which has been brought into contact with a candidate substance suspected to be capable of treating a disease or disorder related to improper sex hormone homeostasis the amount of at least one biomarker selected from any one of Tables 7, 8a, 8b, 9, 10a, 10b, 11a, 11 b, 12a, 12b, 12c, 12d, 13a, 13b, 14a, 14b, 15a, 15b, 15c, 15d, or 16; and
(b) comparing the amounts determined in step (a) to a reference, whereby a substance capable of treating a disease or disorder related to improper sex hormone homeostasis is to be identified.

In a particular embodiment of the method of the invention, a method is provided for identifying a substance for treating a disease or disorder related to improper sex hormone homeostasis comprising:
(a1) (i) selecting a male or female subject;
   (ii) bringing said subject into contact with a compound suspected to be capable of inducing a disease or disorder related to improper sex hormone homeostasis such that a disease or disorder related to improper sex hormone homeostasis is elicited, or
(a2) selecting a male or female suffering from a disease or disorder related to improper sex hormone homeostasis;
(b) obtaining a test sample from said selected subject;
(c) pre-treating said sample in preparation for analysis;
(d) determining the amount of at least one biomarker selected from any one of Tables 7, 8a, 8b, 9, 10a, 10b, 11a, 11 b, 12a, 12b, 12c, 12d, 13a, 13b, 14a, 14b, 15a, 15b, 15c, 15d or 16 in said test sample, and
(e) comparing the amounts determined in step (d) to a reference; and
(f) based on the comparison of step (e), identifying and selecting the substance for treating a disease or disorder related to improper sex hormone homeostasis.

In a preferred embodiment of the aforementioned method said reference is derived from (i) a subject or group of subjects which suffers from a disease or disorder related to improper sex hormone homeostasis or (ii) a subject or group of subjects which has been brought into contact with at least one compound selected from the group consisting of: 17-alpha-Ethynylestradiol, 17-alpha-Methyltestosterone, Bromocriptine Mesylate, Buserelin acetate s.c., Cabergoline, Cyproteron Acetate, Diethanolamine, Diethylstilboestrol dipropionate, Fluoxetine hydrochloride, Flutamide, Genistein, Ifosfamide, Iprodione, Leuprolide acetate salt, Medroxyprogesterone acetat, Metazachlor, Methimazole, Mibolerone, Naphthylisothiocyanate, Norethindrone acetate, Raloxifene Hydrochloride, Tamoxifen, Trenbolone, Triticonazole, and Vinclozolin. In a more preferred embodiment of said method amounts for the biomarkers which differ in the test sample and the reference are indicative for a substance capable of treating a disease or disorder related to improper sex hormone homeostasis.

In another preferred embodiment of the aforementioned method said reference is derived from (i) a subject or group of subjects known to not suffer from a disease or disorder related to improper sex hormone homeostasis or (ii) a subject or group of subjects which has not been brought into contact with at least one compound selected from the group consisting of: 17-alpha-Ethynylestradiol, 17-alpha-Methyltestosterone, Bromocriptine Mesylate, Buserelin acetate s.c., Cabergoline, Cyproteron Acetate, Diethanolamine, Diethylstilboestrol dipropionate, Fluoxetine hydrochloride, Flutamide, Genistein, Ifosfamide, Iprodione, Leuprolide acetate salt, Medroxyprogesterone acetat, Metazachlor, Methimazole, Mibolerone, Naphthylisothiocyanate, Norethindrone acetate, Raloxifene Hydrochloride, Tamoxifen, Trenbolone, Triticonazole, and Vinclozolin. In a more preferred embodiment of the said methods essentially identical amounts for the biomarkers in the test sample and the reference are indicative for a substance capable of treating a disease or disorder related to improper sex hormone homeostasis.

In yet another preferred embodiment of the aforementioned method said reference is a calculated reference for the biomarkers in a population of subjects. In a more preferred embodiment of the said methods essentially identical amounts for the biomarkers in the test sample and the reference are indicative for a substance capable of treating a disease or disorder related to improper sex hormone homeostasis.

The present invention also relates to the use of at least one biomarker selected from any one of Tables 7, 8a, 8b, 9, 10a, 10b, 11a, 11b, 12a, 12b, 12c, 12d, 13a, 13b, 14a, 14b, 15a, 15b, 15c, 15d, or 16 or a detection agent for the said biomarker for diagnosing a disease or disorder related to improper sex hormone homeostasis in a sample of a subject.

Moreover, the present invention relates to a device for diagnosing a disease or disorder related to improper sex hormone homeostasis in a sample of a subject suspected to suffer therefrom comprising:
(a) an analyzing unit comprising a detection agent for at least one biomarker selected from any one of Tables 7, 8a, 8b, 9, 10a, 10b, 11a, 11 b, 12a, 12b, 12c, 12d, 13a, 13b, 14a, 14b, 15a, 15b, 15c, 15d, or 16 which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby a disease or disorder related to improper sex hormone homeostasis is diagnosed.

In a preferred embodiment of the device of the invention said stored reference is a reference derived from a subject or a group of subjects known to suffer from a disease or disorder related to improper sex hormone homeostasis or a subject or group of subjects which has been brought into contact with at least one compound selected from the group consisting of 17-alpha-Ethynylestradiol, 17-alpha-Methyltestosterone, Bromocriptine Mesylate, Buserelin acetate s.c., Cabergoline, Cyproteron Acetate, Diethanolamine, Diethylstilboestrol dipropionate, Fluoxetine hydrochloride, Flutamide, Genistein, Ifosfamide, Iprodione, Leuprolide acetate salt, Medroxyprogesterone acetat, Metazachlor, Methimazole, Mibolerone, Naphthylisothiocyanate, Norethindrone acetate, Raloxifene Hydrochloride, Tamoxifen, Trenbolone, Triticonazole, and Vinclozolin, and said data processor executes instructions for comparing the amount of the at least one biomarker determined by the analyzing unit to the stored reference, wherein an essentially identical amount of the at least one biomarker in the test sample in comparison to the reference is indicative for the presence of a disease or disorder related to improper sex hormone homeostasis or wherein an amount of the at least one biomarker in the test sample which differs in comparison to the reference is indicative for the absence of a disease or disorder related to improper sex hormone homeostasis.

In another preferred embodiment of the device of the invention said stored reference is a reference derived from a subject or a group of subjects known to not suffer from a disease or disorder related to improper sex hormone homeostasis or a subject or group of subjects which has not been brought into contact with at least one compound selected from the group consisting of 17-alpha-Ethynylestradiol, 17-alpha-Methyltestosterone, Bromocriptine Mesylate, Buserelin acetate s.c., Cabergoline, Cyproteron Acetate, Diethanolamine, Diethylstilboestrol dipropionate, Fluoxetine hydrochloride, Flutamide, Genistein, Ifosfamide, Iprodione, Leuprolide acetate salt, Medroxyprogesterone acetat, Metazachlor, Methimazole, Mibolerone, Naphthylisothiocyanate, Norethindrone acetate, Raloxifene Hydrochloride, Tamoxifen, Trenbolone, Triticonazole, and Vinclozolin, and said data processor executes instructions for comparing the amount of the at least one biomarker determined by the analyzing unit to the stored reference, wherein an amount of the at least one biomarker in the test sample which differs in comparison to the reference is indicative for the presence of a disease or disorder related to improper sex hormone homeostasis or wherein an essential identical amount of the at least one biomarker in the test sample in comparison to the reference is indicative for the absence of a disease or disorder related to improper sex hormone homeostasis.

Further, the present invention relates to a kit for diagnosing a disease or disorder related to improper sex hormone homeostasis comprising a detection agent for the at least one biomarker selected from any one of Tables 7, 8a, 8b, 9, 10a, 10b, 11a, 11 b, 12a, 12b, 12c, 12d, 13a, 13b, 14a, 14b, 15a, 15b, 15c, 15d, or 16 and standards for the at least one biomarker the concentration of which is derived from a subject or a group of subjects known to suffer from a disease or disorder related to improper sex hormone homeostasis or derived from a subject or a group of subjects known to not suffer from a disease or disorder related to improper sex hormone homeostasis.

In particular, the present invention relates to a method for diagnosing a disease or disorder of the endocrine pancreas comprising:
(a) determining the amount of at least one biomarker selected from any one of Tables 17a or 17b in a test sample of a subject suspected to suffer from a disease or disorder of the endocrine pancreas, and
(b) comparing the amounts determined in step (a) to a reference, whereby a disease or disorder of the endocrine pancreas is to be diagnosed.

In a particular embodiment of the method of the invention, a method is provided for diagnosing a disease or disorder of the endocrine pancreas comprising:
(a) selecting a male or female subject suspected to suffer from a disease or disorder of the endocrine pancreas;
(b) obtaining a test sample from said selected subject;
(c) pre-treating said sample in preparation for analysis;
(d) determining the amount of at least one biomarker selected from any one of Tables 17a, or 17b in said test sample, and
(e) comparing the amounts determined in step (d) to a reference; and
(f) based on the comparison of step (e), diagnose a disease or disorder of the endocrine pancreas by monitoring, confirmation or classification of the a disease or disorder of the endocrine pancreas or its symptoms.

In a preferred embodiment of the aforementioned method said subject has been brought into contact with a compound suspected to be capable of inducing a disease or disorder of the endocrine pancreas.

The present invention also relates to a method of determining whether a compound is capable of inducing a disease or disorder of the endocrine pancreas in a subject comprising:
(a) determining in a sample of a subject which has been brought into contact with a compound suspected to be capable of inducing a disease or disorder of the endocrine pancreas the amount of at least one biomarker selected from any one of Tables 17a or 17b; and
(b) comparing the amounts determined in step (a) to a reference, whereby the capability of the compound to induce a disease or disorder of the endocrine pancreas is determined.

In a particular embodiment of the method of the invention, a method is provided for determining whether a compound is capable of inducing a disease or disorder of the endocrine pancreas in a subject comprising:
(a1) (i) selecting a male or female subject;
   (ii) bringing said subject into contact with a compound suspected to be capable of inducing a disease or disorder of the endocrine pancreas, or
(a2) selecting a male or female subject brought into contact with a compound capable of inducing a disease or disorder of the endocrine pancreas;
(b) obtaining a test sample from said selected subject;
(c) pre-treating said sample in preparation for analysis;
(d) determining the amount of at least one biomarker selected from any one of Tables 17a, or 17b in said test sample, and
(e) comparing the amounts determined in step (d) to a reference; and
(f) based on the comparison of step (e), identifying whether the compound is capable of inducing a disease or disorder of the endocrine pancreas, or not.

In a preferred embodiment of the aforementioned method said compound is at least one compound selected from the group consisting of: Clofibrate, Fluoroglycofen-ethyl, Glipizide, Pioglitazone hydrochloride, Rosiglitazone maleate, Streptozotocin, and Ziprasidone hydrochloride.

In another preferred embodiment of the methods of the present invention said reference is derived from (i) a subject or group of subjects which suffers from a disease or disorder of the endocrine pancreas or (ii) a subject or group of subjects which has been brought into contact with at least one compound selected from the group consisting of: Clofibrate, Fluoroglycofen-ethyl, Glipizide, Pioglitazone hydrochloride, Rosiglitazone maleate, Streptozotocin, and Ziprasidone hydrochloride. In a more preferred embodiment of said method essentially identical amounts for the biomarkers in the test sample and the reference are indicative for a disease or disorder of the endocrine pancreas.

In another preferred embodiment of the methods of the present invention said reference is derived from (i) a subject or group of subjects known to not suffer from a disease or disorder of the endocrine pancreas or (ii) a subject or group of subjects which has not been brought into contact with at least one compound selected from the group consisting of: Clofibrate, Fluoroglycofen-ethyl, Glipizide, Pioglitazone hydrochloride, Rosiglitazone maleate, Streptozotocin, and Ziprasidone hydrochloride. In a more preferred embodiment of said methods amounts for the biomarkers which differ in the test sample in comparison to the reference are indicative for a disease or disorder of the endocrine pancreas.

In yet another embodiment of the methods of the present invention said reference is a calculated reference for the biomarkers for a population of subjects. In a more preferred embodiment of said methods amounts for the biomarkers which differ in the test sample in comparison to the reference are indicative for a disease or disorder of the endocrine pancreas.

The present invention also contemplates a method of identifying a substance for treating a disease or disorder of the endocrine pancreas comprising the steps of:
(a) determining in a sample of a subject suffering from a disease or disorder of the endocrine pancreas which has been brought into contact with a candidate substance suspected to be capable of treating a disease or disorder of the endocrine pancreas the amount of at least one biomarker selected from any one of Tables 17a or 17b; and
(b) comparing the amounts determined in step (a) to a reference, whereby a substance capable of treating a disease or disorder of the endocrine pancreas is to be identified.

In a particular embodiment of the method of the invention, a method is provided for identifying a substance for treating a disease or disorder of the endocrine pancreas comprising:
(a1) (i) selecting a male or female subject;
   (ii) bringing said subject into contact with a compound suspected to be capable of inducing a disease or disorder of the endocrine pancreas such that a disease or disorder of the endocrine pancreas is elicited, or
(a2) selecting a male or female suffering from a disease or disorder of the endocrine pancreas;
(b) obtaining a test sample from said selected subject;
(c) pre-treating said sample in preparation for analysis;
(d) determining the amount of at least one biomarker selected from any one of Tables 17a, or 17b in said test sample, and
(e) comparing the amounts determined in step (d) to a reference; and
(f) based on the comparison of step (e), identifying and selecting the substance for treating a disease or disorder of the endocrine pancreas.

In a preferred embodiment of the aforementioned method said reference is derived from (i) a subject or group of subjects which suffers from a disease or disorder of the endocrine pancreas or (ii) a subject or group of subjects which has been brought into contact with at least one compound selected from the group consisting of: Clofibrate, Fluoroglycofen-ethyl, Glipizide, Pioglitazone hydrochloride, Rosiglitazone maleate, Streptozotocin, and Ziprasidone hydrochloride. In a more preferred embodiment of said method amounts for the biomarkers which differ in the test sample and the reference are indicative for a substance capable of treating a disease or disorder of the endocrine pancreas.

In another preferred embodiment of the aforementioned method said reference is derived from (i) a subject or group of subjects known to not suffer from a disease or disorder of the endocrine pancreas or (ii) a subject or group of subjects which has not been brought into contact with at least one compound selected from the group consisting of: Clofibrate, Fluoroglycofen-ethyl, Glipizide, Pioglitazone hydrochloride, Rosiglitazone maleate, Streptozotocin, and Ziprasidone hydrochloride. In a more preferred embodiment of the said methods essentially identical amounts for the biomarkers in the test sample and the reference are indicative for a substance capable of treating a disease or disorder of the endocrine pancreas.

In yet another preferred embodiment of the aforementioned method said reference is a calculated reference for the biomarkers in a population of subjects. In a more preferred embodiment of the said methods essentially identical amounts for the biomarkers in the test sample and the reference are indicative for a substance capable of treating a disease or disorder of the endocrine pancreas.

The present invention also relates to the use of at least one biomarker selected from any one of Tables 17a or 17b or a detection agent for the said biomarker for a disease or disorder of the endocrine pancreas in a sample of a subject.

Moreover, the present invention relates to a device for diagnosing a disease or disorder of the endocrine pancreas in a sample of a subject suspected to suffer therefrom comprising:
(a) an analyzing unit comprising a detection agent for at least one biomarker selected from any one of Tables 17a or 17b which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby a disease or disorder of the endocrine pancreas is diagnosed.

In a preferred embodiment of the device of the invention said stored reference is a reference derived from a subject or a group of subjects known to suffer a disease or disorder of the endocrine pancreas or a subject or group of subjects which has been brought into contact with at least one compound selected from the group consisting of Clofibrate, Fluoroglycofen-ethyl, Glipizide, Pioglitazone hydrochloride, Rosiglitazone maleate, Streptozotocin, and Ziprasidone hydrochloride, and said data processor executes instructions for comparing the amount of the at least one biomarker determined by the analyzing unit to the stored reference, wherein an essentially identical amount of the at least one biomarker in the test sample in comparison to the reference is indicative for the presence of a disease or disorder of the endocrine pancreas or wherein an amount of the at least one biomarker in the test sample which differs in comparison to the reference is indicative for the absence of a disease or disorder of the endocrine pancreas.

In another preferred embodiment of the device of the invention said stored reference is a reference derived from a subject or a group of subjects known to not suffer from a disease or disorder of the endocrine pancreas or a subject or group of subjects which has not been brought into contact with at least one compound selected from the group consisting of Clofibrate, Fluoroglycofen-ethyl, Glipizide, Pioglitazone hydrochloride, Rosiglitazone maleate, Streptozotocin, and Ziprasidone hydrochloride, and said data processor executes instructions for comparing the amount of the at least one biomarker determined by the analyzing unit to the stored reference, wherein an amount of the at least one biomarker in the test sample which differs in comparison to the reference is indicative for the presence of a disease or disorder of the endocrine pancreas or wherein an essential identical amount of the at least one biomarker in the test sample in comparison to the reference is indicative for the absence of a disease or disorder of the endocrine pancreas.

Further, the present invention relates to a kit for diagnosing a disease or disorder of the endocrine pancreas comprising a detection agent for the at least one biomarker selected from any one of Tables 17a or 17b and standards for the at least one biomarker the concentration of which is derived from a subject or a group of subjects known to suffer from a disease or disorder of the endocrine pancreas or derived from a subject or a group of subjects known to not suffer from a disease or disorder of the endocrine pancreas.

In particular the present invention contemplates also the following specific methods, uses, devices and kits.

The following definitions and explanations apply mutatis mutandis to all the previous embodiments of the present invention as well as the embodiments described in the following.

The methods referred to in accordance with the present invention may essentially consist of the aforementioned steps or may include further steps. Further steps may relate to sample pre-treatment or evaluation of the diagnostic results obtained by the methods. Preferred further evaluation steps are described elsewhere herein. The methods may partially or entirely be assisted by automation. For example, steps pertaining to the determination of the amount of a biomarker can be automated by robotic and automated reader devices. Likewise, steps pertaining to a comparison of amounts can be automated by suitable data processing devices, such as a computer, comprising a program code which when being executed carries out the comparison automatically. A reference in such a case will be provided from a stored reference, e.g., from a database. It is to be understood that the method is, preferably, a method carried out ex vivo on a sample of a subject, i.e. not practised on the human or animal body.

The term “diagnosing” as used herein refers to assessing the probability according to which a subject is suffering from a condition, such as intoxication, disease or disorder referred to herein, or has a predisposition for such a condition. Diagnosis of a predisposition may sometimes be referred to as prognosis or prediction of the likelihood that a subject will develop the condition within a predefined time window in the future. As will be understood by those skilled in the art, such an assessment, although preferred to be, may usually not be correct for 100% of the subjects to be diagnosed. The term, however, requires that a statistically significant portion of subjects can be identified as suffering from the condition or having a predisposition for the condition. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% or at least 95%. The p-values are, preferably, 0.2, 0.1, 0.05.

Diagnosing according to the present invention also includes monitoring, confirmation, and classification of a condition or its symptoms as well as a predisposition therefor. Monitoring refers to keeping track of an already diagnosed condition or predisposition. Monitoring encompasses, e.g., determining the progression of the condition or predisposition, determining the influence of a particular treatment on the progression of the condition or the influence of prophylactic measures such as a prophylactic treatment or diet on the development of the condition in a subject having a predisposition. Said treatment, prophylactic measure or diet may be adjusted and the influence of the adjustment may be investigated as an aspect of the monitoring. Moreover, if progression of the condition or a predisposition therefor is monitored, said monitoring may also include determining a monitoring frequency and to recommend and/or carry out additional monitoring measures such as measurement of additional biochemical or other health parameters. Confirmation relates to the strengthening or substantiating a diagnosis of the condition or a predisposition for the condition already determined using other indicators or markers. Confirmation may also include in an aspect the administration or adaptation of therapeutic measures based on the confirmed condition or predisposition therefor. Classification relates to (i) allocating the condition into different classes, e.g., corresponding to the strength of the symptoms accompanying the condition, or (ii) differentiating between different stages, disease or disorders accompanying the condition. Classification may also include in an aspect the administration or adaptation of therapeutic measures based on the classified condition, symptoms or predisposition therefor. A predisposition for the condition can be classified based on the degree of the risk, i.e. the probability according to which a subject will develop the condition later. Moreover, classification also, preferably, includes allocating a mode of action to a compound to be tested by the methods of the present invention. Specifically, the methods of the present invention allow for determination of a specific mode of action of a compound for which such mode of action is not yet known. This is, preferably, achieved by comparing the amount determined for the at least one biomarker or a biomarker profile representative for said compound to the amount of the biomarker or biomarker profile determined for a compound for which the mode of action is known as a reference. The classification of the mode of action allows an even more reliable assessment of toxicity of a compound because the molecular targets of the compound are identified. The methods of the present invention aiming at diagnosing a disease or condition may be used for screening compounds for toxicological effects and reporting thereon as well as in compound development, e.g., in increasing safety or in developing drugs or identifying effective concentrations.

In accordance with the present invention, a compound can also be identified as being capable of inducing endocrine disease or disorder. Such identification, preferably, also includes making suggestions for the manufacture, handling, storage and/or transport of the compound and its applications. Such suggestions include establishing safety protocols for manufacture, handling, storage, transport and/or application, labelling the compound according to its toxicity potential, limiting exposure to humans, animals and/or to the environment. Moreover, if a compound is identified as eliciting endocrine disease or disorder, safety levels such as LD50/LC50 and/or ED50/EC50 values and derived thresholds are, preferably, determined.

The term “an endocrine disease or disorder” as used herein relates to any damage or impairment of an organ or cells of the endocrine system which results in an impaired endocrine function, in particular, impaired adrenal hormone homeostasis or function, sex hormone homeostasis or function or pancreatic hormone homeostasis or function. Preferably, affected by an endocrine disease or disorder are the adreanl cortex, sex hormone producing organs and/or the pancreas. Accordingly, the term an endocrine disease or disorder as used herein encompasses toxicity of the adrenal cortex, diseases and disorders related to improper sex hormone homeostasis, and/or diseases and disorders of the endocrine pancreas, in general. Preferably, an endocrine disease or disorder as used herein is induced by or is the result of the administration of a chemical compound or drug, i.e. so-called toxin-induced endocrine disease or disorder.

The symptoms and clinical signs of the aforementioned manifestations of an endocrine disease or disorder are well known to the person skilled in the art and are described in detail in standard books of toxicology, e.g., H. Marquardt, S. G. Schafer, R. O. McClellan, F. Welsch (eds.), “Toxicology”, Chapter 13: The Liver, 1999, Academic Press, London.

The term “toxicity of the adrenal cortex” used herein refers, preferably, to an impairment of the function of the adrenal cortex. Preferably, toxicity of the adrenal cortex is acute toxicity of the adrenal cortex or chronic toxicity of the adrenal cortex. There are a variety of spontaneous lesions in the adrenal cortex that must be differentiated from chemically induced lesions considering the mechanisms of toxicity. Acute toxicity of the adrenal cortex can have multiple morphologic manifestations as a consequence of impaired steroid genesis. Chronic toxicity of the adrenal cortex can lead to atrophy, nodular regeneration, fibrosis, or primary proliferation of cortical cells. The reason the adrenal cortex is predisposed to the toxic effects of xenobiotic chemicals appears to be related to at least two factors. First, adrenal cortical cells of most animal species contain large stores of lipids used primarily as substrate for steroid genesis. Many adrenal cortical toxic compounds are lipophilic and therefore can accumulate in these lipid-rich cells. Second, adrenal cortical cells have enzymes capable of metabolizing of xenobiotic chemicals, including enzymes of the cytochrome P450 family. A number of toxic xenobiotic chemicals serve as pseudo substrates for these enzymes and can be metabolized to reactive toxic compounds. These reactive compounds subsequently cause direct toxic effects.

Preferably, the at least one biomarker to be determined by the methods of the present invention is selected from any one of Tables 1a, 1 b, 2a, 2b, 3a, 3b, 4a, 4b, 4c, 4d, 5a, 5b, 6a or 6b if the an endocrine disease or disorder is toxicity of the adrenal cortex. More preferably, said toxicity of the adrenal cortex is an impaired adrenal antiglucocorticoid homeostasis, adreanl cortex steroid syntheses inhibition, adrenal aromatase inhibiton or adrenal hypocortisolism.

More preferably, said toxicity of the adrenal cortex is characterized by impaired adrenal antiglucocorticoid homeostasis if the at least one biomarker is selected from the biomarkers shown in Table 1a or 1b.

More preferably, said toxicity of the adrenal cortex is characterized by adreanl cortex steroid syntheses inhibition if the at least one biomarker is selected from the biomarkers shown in Table 2a, 2b, 5a, 5b, 6a or 6b.

Also more preferably, said toxicity of the adrenal cortex is characterized by an adrenal aromatase inhibiton if the at least one biomarker is selected from the biomarkers shown in Table 3a or 3b.

More preferably, said toxicity of the adrenal cortex is characterized by adrenal hypocortisolism if the at least one biomarker is selected from the biomarkers shown in Table 4a, 4b, 4c, or 4d.

The term “disorder or disease related to improper sex hormone homeostasis” as used herein, preferably, refers to an impairment of the sex hormone homeostasis and, thus, an impairment of the sex hormone producing tissues. The term “sex hormones” as used herein refers to androgens and estrogens.

The physiologic balance between different sex steroid hormones is crucial for the development, maintenance, and function of the reproductive system as well as for the differentiation of the sexual phenotype during ontogeny. Estrogens (estrone and estradiol) are products of the androgens (androstenedione and testosterone), and the reaction is catalyzed by aromatase. Therefore, disturbances in aromatase expression and/or changes in its catalytic activity are expected to exhibit negative effects on reproduction parameters. Improper sex steroid hormone synthesis, preferably, effects fertility, sexual behaviour, and reproductive organ development. Moreover, inhibition of aromatase may, preferably, cause an increase in joint disorders; an increase in the incidence of osteoporosis and fractures, hypercholesterolemia and osteonecrosis, decreased rate of bone maturation and growth, decreased sperm production, infertility, aggressive behaviour, adrenal insufficiency, kidney failure and liver dysfunction.

Androgens are a group of hormones that stimulates or controls the development and maintenance of male characteristics by binding to androgen receptors that primarily influence the growth and development of the male reproductive system including the activity of the accessory male sex organs and development of male secondary sex characteristics. Androgens are also the original anabolic steroids and the precursor of all estrogens, the female sex hormones. The predominant and most active androgen is testosterone, which is produced by the interstitial Leydig cells of the testes. The actual secretion of androgens by these cells is controlled by luteinizing hormone (LH) from the pituitary gland. The other androgens, which support the functions of testosterone, are produced mainly by the adrenal cortex and only in relatively small quantities includes any of the 19-carbon steroids synthesized by the adrenal cortex, the inner portion of the adrenal gland, that function as weak steroids or steroid precursors, including dehydroepiandrosterone (DHEA), dehydroepiandrosterone sulfate (DHEA-S), androstenedione, androstenediol, androsterone, and dihydrotestosterone (DHT). Testosterone is the principal androgen in males, secreted in the response to luteinizing hormone released from the pituitary gland, can directly bind to androgen receptors and is converted in some tissues to dihydrotestosterone, which also binds to androgen receptors. Activation of androgen receptors results in differentiation of the external genitalia, increased hair growth during puberty, and stimulation of the prostate gland. Testosterone also contributes to the mass and strength of skeletal muscle. Testosterone is also converted to estrogen, which then binds to estrogen receptors and mediates closure of the epiphyses in the bone. In general, an androgen antagonist (anti-androgen) is any of a group of hormone receptor antagonist compounds that are capable of preventing or inhibiting the biologic effects on normally responsive tissues in the body. Anti-androgens usually work by blocking the appropriate receptors, competing for binding sites on the cell's surface, obstructing the androgens' pathway. Xenobiotic chemicals acting as androgen receptor antagonists can disrupt the hypothalamic-pituitary-testis axis at one of several possible sites interfering with negative feedback control, resulting in hyperfunction of the pituitary gland, such as procymidone due to binding to the androgen receptor, increases circulating levels of LH and results in stimulation of Leydig cells, leading to an increased incidence of hyperplasia and adenomas in rats. Another indicator of reduced plasma testosterone levels is the size of the seminal vesicles and prostate. Their secretory function is androgen dependent and very sensitive to circulating concentrations of testosterone. However, secretory function may also be affected by inhibitors of 5-alpha reductase, which metabolizes testosterone to dihydrotestosterone. Androgen-receptor antagonists can be used in combination with a gonadotropin-releasing hormone (GnRH) analog in the treatment of metastatic prostate cancer. Environmental chemicals that have been shown to act as androgen receptor antagonists include the metabolites of the agricultural fungicide vinclozolin, the DDT metabolite DDE, some hydroxylated PCBs, and the organophosphate insecticide fenitrothion. The consequence of androgen receptor antagonism is typically considered demasculinization and reductions in the size of the ventral prostate and seminal vesicle weights along with deformities of the penis. In addition, chemicals can also act as receptor agonists and stimulate receptor-dependent physiological processes in the absence of the endogenous receptor hormone. Such inappropriate stimulation can result in the errant expression of hormone-dependent processes such as gynecomastia in males.

Estrogens are a group of hormones that primarily influence the female reproductive tract in its development, maturation, and function. While estrogens are present in both male and female, they are usually present at significantly higher levels in women of reproductive age. There are three major estrogenic hormones, estradiol as the predominant one and estrone and estriol. The major sources of estrogens are the ovaries and the placenta; additional small amounts are secreted by the adrenal glands, the liver, the breast and by the male testes. The follicle and interstitial cells in the ovaries are the predominant production sites of estrogens in the female. Estrogen levels are highest during ovulation and after menstruation, when the corpus luteum replaces the empty follicle. Androgens are converted to estrogens by an aromatase. The ovaries are the richest source of aromatase. Estradiol, the most potent estrogen, is synthesized from testosterone. Estrone can be formed from estradiol, but its major precursor is androstenedione. Estriol, the weakest of the estrogens, is formed from both estrone and estradiol. Estrogens bind reversibly to a protein known as sex hormone-binding globulin and at its target tissues binds to the estrogen receptor. In females, estrogens affect the ovaries, vagina, fallopian tubes, uterus, and mammary glands. Estrogens influence the structural differences between the male and female bodies. In the male, traces of estrogens are present in the blood and urine; estrogens seem to be most evident in the male during puberty and old age. In males, estrogens regulate certain functions of the reproductive system important to the maturation of sperm and are necessary for a healthy libido. Estrogens coordinate the growth and maintenance of the reproductive tract, pituitary, breasts, and other tissues. Estrogens are also responsible for maturation of the skeleton and development of female secondary sex characteristics when females enter puberty. The other important functions of estrogens include moduletion of many metabolic processes, e.g., hepatic metabolism. The production of sex hormone-binding globulin, thyroxine-binding globulin, blood-clotting factors and plasminogen in the liver is stimulated by estrogens. Estrogens stimulate cellular proliferation, induce RNA and protein synthesis of uterine and ovary tissues, and increase the size of the cells. This effect leads to the growth and regeneration of the endometrial layer and increase in the number and size of endometrial glands. Under the influence of estrogen, vaginal mucosa becomes thicker, as cervical mucus becomes thinner. Chemicals often can function as either a receptor agonist or antagonist depending on the level of endogenous hormone. A weak agonist may bind to a receptor and stimulate some low-level receptor-mediated activity in the absence of the endogenous hormone. However, in the presence of the hormone, binding of the xenobiotic to the receptor may prevent binding of the endogenous hormone, and if the xenobiotic is a much weaker activator of receptor-mediated activity, then the net effect is loss of activity. Thus, in the presence of the endogenous hormone, the xenobiotic functions as a receptor antagonist. Whether a weak chemical-agonist functions as an agonist or antagonist depends on the concentration, the binding affinity to the receptor, the concentration of the endogenous hormone to the receptor, and the binding affinity of the endogenous hormone to the receptor. For example, the drug tamoxifen functions as an estrogen receptor antagonists in reproductive tissue but functions as an agonist with respect to the preservation of bone mineral density and reducing serum cholesterol concentrations. Accordingly tamoxifen can function as a prophylactic against the growth of estrogen-responsive breast cancers and osteoporosis via antagonism and agonism, respectively. Other drugs that bind to the estrogen receptor as an antagonist or mixed agonist/antagonist include raloxifene. Environmental estrogen receptor antagonists include some phytochemicals and PCBs. Consequences of estrogen receptor antagonism are typically considered de-feminization. In laboratory animal studies, estrogen receptor antagonists have been shown in females to disrupt estrous cycles, impair fertility, increase preimplantation loss, and cause embryolethality. Gynecomastia is a common side effect of estrogenic drugs such as DES and fosfestrol when administered to adult males. The physiological consequences of chemical-estrogenic activity is typically characteristic of feminization, that is, the acquisition of female characteristics. Among the steroid hormone receptors, the estrogen receptor appears most susceptible to the agonistic action of xenobiotics. Estrogen receptor agonists are quite diverse in molecular structure but it is not clear why the estrogen receptor would be more susceptible to the agonistic action of xenobiotics as compared to other steroid hormone receptors. The estrogen receptor is often referred to as a promiscuous receptor because of this susceptibility to agonistic interactions with xenobiotics.

Preferably, the at least one biomarker to be determined by the methods of the present invention is selected from any one of Tables 7, 8a, 8b, 9, 10a, 10b, 11a, 11 b, 12a, 12b, 12c, 12d, 13a, 13b, 14a, 14b, 15a, 15b, 15c, 15d or 16 if the an endocrine disease or disorder is disorder or disease related to improper sex hormone homeostasis.

More preferably, said disorder or disease related to improper sex hormone homeostasis is characterized by impaired estrogen function if the at least one biomarker is selected from the biomarkers shown in Table 7, 12a, 12b, 12c, 12d.

More preferably, said disorder or disease related to improper sex hormone homeostasis is characterized by impaired estrogen receptor modulation if the at least one biomarker is selected from the biomarkers shown in Table 8a or 8b.

More preferably, said disorder or disease related to improper sex hormone homeostasis is characterized by GnRH agonist functions if the at least one biomarker is selected from the biomarkers shown in Table 9.

More preferably, said disorder or disease related to improper sex hormone homeostasis is characterized by antiandrogenic function if the at least one biomarker is selected from the biomarkers shown in Table 10a or 10b.

More preferably, said disorder or disease related to improper sex hormone homeostasis is characterized by antiandrogen receptor antagonist function, prferably on prostate, if the at least one biomarker is selected from the biomarkers shown in Table 11a or 11 b.

More preferably, said disorder or disease related to improper sex hormone homeostasis is characterized by anti-prolactin function if the at least one biomarker is selected from the biomarkers shown in Table 13a or 13b.

More preferably, said disorder or disease related to improper sex hormone homeostasis is characterized by impaired sex hormone function if the at least one biomarker is selected from the biomarkers shown in Table 14a or 14b.

More preferably, said disorder or disease related to improper sex hormone homeostasis is characterized by impaired testosterone function if the at least one biomarker is selected from the biomarkers shown in Table 15a, 15b, 15c, or 15d.

The term “disorder or disease of the endocrine pancreas” as used herein, preferably, refers to an impairment of the endocrine pancreas. The pancreas receives regulatory innervation via hormones in the blood and through the autonomic nervous system. These two inputs regulate the secretory activity of the pancreas. Sympathetic (adrenergic) α2 decreases secretion from beta cells, increases and secretion from alpha cells while parasympathetic (muscarinic) M3 increases stimulation of alpha cells and beta cells. The principal function of the endocrine pancreas is the secretion of insulin and other polypeptide hormones necessary for the cellular storage or mobilization of glucose, amino acids, and triglycerides. Islet function may be regulated by signals initiated by autonomic nerves, circulating metabolites, circulating hormones, or local hormones. The importance of the endocrine pancreas is that insulin plays a central role in the regulation of energy metabolism. A relative or absolute deficiency of insulin leads to diabetes mellitus. Insulin stimulates the transport of glucose into tissues, the transport of amino acids into tissues, and the transport of fatty acids into tissues. In the fasting state, insulin secretion decreases and glucagon secretion increases. Liver glycogen stores, followed later by protein and fat stores, are mobilized to produce glucose. Ultimately, most nutrient needs are provided by fatty acids mobilized from fat stores. The pancreatic hormone glucagon also plays a key role in maintaining glucose homeostasis and in regulating nutrient storage. An adequate supply of glucose is required for optimal body growth and development and for the function of the central nervous system, for which glucose is the major source of energy. Intoxication of the pancreas will effect its endocrine functions. The cellular effects of an intoxication span from acute injury and death, to hyperplasia, metaplasia and malignant transformation. In addition, therapeutic drugs are toxic for the pancreas and comprised of azathioprine, estrogens, furosemide, methyldopa, pentamidine, procainamide, sulfonamides, and thiazide diuretics. From the above it is evident that the endocrine pancreas can be influenced and impaired at different levels and by different stimuli. Besides genetic influences, exogenous stimuli such as xenobiotic chemicals may impair endocrine pancreas function, especially with regards to hormone homeostasis.

Preferably, the at least one biomarker to be determined by the methods of the present invention is selected from any one of Tables 17a or 17b if the an endocrine disease or disorder is a disorder or disease of the endocrine pancreas.

It was found in accordance with the present invention that a combination of more than one of the biomarkers listed in any of the aforementioned Tables further strengthen the diagnosis since each of the biomarkers is an apparently statistically independent predictor for the diagnosis. Moreover, the specificity for an endocrine disease or disorder is also significantly increased since influences from other tissues on the marker abundance are counterbalanced. Thus, the term “at least one” as used herein, preferably, refers to a combination of at least 2, at least 3, at least 4, at least 5, at least 6, at least 7, at least 8, at least 9 or at least 10 of the biomarkers referred to in any one of the accompanying Tables. Preferably, all biomarkers recited in any one of the Tables are to be determined in combination in accordance with the methods of the present invention.

Preferred groups or combinations of biomarkers for an endocrine disease or disorder from the individual tables and for the indications referred to in the tables are as follows:

Tables 1a and 1b (Adrenal antiglucocorticoid): Citrulline, Cytosine, dihomo-gamma-Linolenic acid (C20:cis[8, 11, 14]3), Uric acid, Ceramide (d18:1, C24:0) or Ascorbic acid.

Tables 2a and 2b (Adrenal Cortex Steroid synthese inhibition): Tricosanoic acid (C23:0), Ceramide (d18:1, C24:1), trans-4-Hydroxyproline, 18-Hydroxy-11-deoxycorticosterone or alpha-Tocopherol.

Tables 3a and 3b (Adrenals aromatase inhibitor): Threonic acid, Lysophosphatidylcholine (C20:4) or Glucose.

Tables 4a and 4b (Adrenals Hypocortisolism female): Choline plasmalogen No 03, Biotin, alpha-Tocopherol, Citrate, 5-Oxoproline or Ketoleucine.

Tables 4c and 4d (Adrenals Hypocortisolism male): Lysophosphatidylethanolamine (C22:5), 3,4-Dihydroxyphenylalanine (DOPA), Phosphatidylcholine No 04, Biotin or 21-Hydroxyprogesterone (11-Deoxycorticosterone).

Tables 5a and 5b (adrenals cortex synth. inhibitors): 3-O-Methylsphingosine (d18:1), threo-Sphingosine (d18:1), erythro-Sphingosine (d18:1), 5-O-Methylsphingosine (d18:1) or Nervonic acid (C24:cis[15]1).

Tables 6a and 6b (Adrenals Steroid synth. Inhibitors): TAG (C18:2, C18:3), TAG (C18:3, C18:2, C18:1), Linoleic acid (C18:cis[9,12]2), Glycerol, lipid fraction or TAG (C18:1, C18:2).

Table 7 (Hormone (estronenic)): Behenic acid (C22:0), Arachidonic acid (C20:cis[5, 8, 11, 14]4), Nervonic acid (C24:cis[15]1), erythro-Sphingosine (d18:1) or myo-Inositol, lipid fraction.

Tables 8a and 8b (Hormones (Estrogen Rez. Modulator)): Phosphatidylcholine (C18:0, C20:3), Coenzyme Q9, Lysophosphatidylcholine (C17:0), TAG (C18:2, C18:3) or Proline.

Tables 9 (Hormones (GnRH agonist)): Testosterone, Androstenedione, Uric acid, Glycine or Heptadecanoic acid (C17:0).

Tables 10a and 10b (Hormones (antiandrogen)): trans-4-Hydroxyproline, threo-Sphingosine (d18:1), 3-O-Methylsphingosine (d18:1), Lignoceric acid (C24:0) or erythro-Sphingosine (d18:1).

Tables 11a 11b (homones (antiandrogen receptor antagonist prostate)): Tryptophan, Threonine, Lignoceric acid (C24:0), Nervonic acid (C24:cis[15]1) or Behenic acid (C22:0).

Tables 12a and 12b (Hormones (estrogens female)): Proline, Choline plasmalogen (C18, C20:4), Phosphatidylcholine (C18:0, C20:4), Sphingomyelin (d18:2, C18:0) or Choline plasmalogen No 02.

Tables 12c and 12d (Hormones (estrogens male)): Sphingomyelin (d18:2, C16:0), Choline plasmalogen No 02, Mannose, Lysophosphatidylcholine (C18:0) or Lysophosphatidylethanolamine (C22:0).

Tables 13a and 13b (Hormones (putative anti-prolactin)): Ceramide (d18:1, C24:1), Cholesterolester No 01, myo-Inositol-2-phosphate, lipid fraction, Phosphatidylcholine (C18:0, C20:3) or Ceramide (d18:1, C24:0).

Tables 14a and 14b (Hormones (Sexhormone)): Choline plasmalogen No 02, Cholesterol, erythro-Sphingosine (d18:1), Lignoceric acid (C24:0) or Sphingomyelin (d18:2, C16:0).

Tables 15a and 15b (hormones (testosterone female)): Lysine, trans-4-Hydroxyproline, Phosphatidylcholine (C16:0/C22:6), Glycine or 3-Hydroxybutyrate.

Tables 15c and 15d (Hormones (testosterone)): Kynurenic acid, Lysophosphatidylethanolamine (C22:5), Sphingomyelin (d18:1, C24:0), Homovanillic acid (HVA) or Phosphate, lipid fraction.

Table 16 (hormones (testosterone reduction prostate)): Phosphatidylcholine (C18:1, C18:2), Pantothenic acid, Tyrosine, 3,4-Dihydroxyphenylacetic acid (DOPAC) or Androstenedione.

Tables 17a and 17b (Pancreas endocrine modulation): Valine, Alanine, Proline, Ketoleucine or Serine.

Thus, preferably, the at least one biomarker is at least one biomarker selected from the aforementioned group or the at least one biomarker is a combination of biomarkers consisting or comprising the aforementioned group of biomarkers. The aforementioned biomarkers and combinations of biomarkers have been identified as key biomarkers having a particular high diagnostic value as described in more detail in the accompanying Examples.

Furthermore, other biomarkers or clinical parameters including known metabolites, genetic mutations, transcript and/or protein amounts or enzyme activities may still be determined in addition. Such, additional clinical or biochemical parameters which may be determined in accordance with the method of the present invention are well known in the art.

The term “biomarker” as used herein refers to a chemical compound whose presence or concentration in a sample is indicative for the presence or absence or strength of a condition, preferably, an endocrine disease or disorder as referred to herein. The chemical compound is, preferably, a metabolite or an analyte derived therefrom. An analyte is a chemical compound which can be identical to the actual metabolite found in an organism. However, the term also includes derivatives of such metabolites which are either endogenously generated or which are generated during the isolation or sample pre-treatment or as a result of carrying out the methods of the invention, e.g., during the purification and/or determination steps. In specific cases the analyte is further characterized by chemical properties such as solubility. Due to the said properties, the analyte may occur in polar or lipid fractions obtained during the purification and/or determination process. Thus, chemical properties and, preferably, the solubility shall result in the occurrence of an analyte in either polar or lipid fractions obtained during the purification and/or determination process. Accordingly, the said chemical properties and, in particular the solubility taken into account as the occurrence of an analyte in either polar or lipid fractions obtained during the purification and/or determination process shall further characterize the analyte and assist in its identification. Details on how these chemical properties can be determined and taken into account are found in the accompanying Examples described below. Preferably, the analyte represents the metabolite in a qualitative and quantitative manner and, thus, allows inevitably concluding on the presence or absence or the amount of the metabolite in a subject or at least in the test sample of said subject. Biomarker, analyte and metabolite are referred to herein in the singular but also include the plurals of the terms, i.e. refer to a plurality of biomarker, analyte or metabolite molecules of the same molecular species. Moreover, a biomarker according to the present invention is not necessarily corresponding to one molecular species. Rather, the biomarker may comprise stereoisomers or enantiomers of a compound. Further, a biomarker can also represent the sum of isomers of a biological class of isomeric molecules. Said isomers shall exhibit identical analytical characteristics in some cases and are, therefore, not distinguishable by various analytical methods including those applied in the accompanying Examples described below. However, the isomers will share at least identical sum formula parameters and, thus, in the case of, e.g., lipids an identical chain length and identical numbers of double bonds in the fatty acid and/or sphingo base moieties

The term “test sample” as used herein refers to samples to be used for the diagnosis of an endocrine disease or disorder by the methods of the present invention. Preferably, said test sample is a biological sample. Samples from biological sources (i.e. biological samples) usually comprise a plurality of metabolites. Preferred biological samples to be used in the method of the present invention are samples from body fluids, preferably, blood, plasma, serum, saliva, bile, urine or cerebrospinal fluid, or samples derived, e.g. by biopsy, from cells, tissues or organs, preferably from the liver. More preferably, the sample is a blood, plasma or serum sample, most preferably, a plasma sample. Biological samples are derived from a subject as specified elsewhere herein. Techniques for obtaining the aforementioned different types of biological samples are well known in the art. For example, blood samples may be obtained by blood taking while tissue or organ samples are to be obtained, e.g. by biopsy.

The aforementioned samples are, preferably, pre-treated before they are used for the methods of the present invention. As described in more detail below, said pre-treatment may include treatments required to release or separate the compounds or to remove excessive material or waste. Suitable techniques comprise centrifugation, extraction, fractioning, ultra-filtration, protein precipitation followed by filtration and purification and/or enrichment of compounds. Moreover, other pretreatments are carried out in order to provide the compounds in a form or concentration suitable for compound analysis. For example, if gas-chromatography coupled mass spectrometry is used in the method of the present invention, it will be required to derivatize the compounds prior to the said gas chromatography. Suitable and necessary pre-treatments depend on the means used for carrying out the method of the invention and are well known to the person skilled in the art. Pre-treated samples as described before are also comprised by the term “sample” as used in accordance with the present invention.

The term “subject” as used herein relates to animals, preferably to mammals such as mice, rats, guinea pigs, rabbits, hamsters, pigs, sheep, dogs, cats, horses, monkeys, or cows and, also preferably, to humans. More preferably, the subject is a rodent and, most preferably, a rat. Other animals which may be diagnosed applying the methods of the present invention are fishes, birds or reptiles. Preferably, said subject was in or has been brought into contact with a compound suspected to be capable of inducing an endocrine disease or disorder. A subject which has been brought into contact with a compound suspected to induce an endocrine disease or disorder may, e.g., be a laboratory animal such as a rat which is used in a screening assay for, e.g., toxicity of compounds. A subject suspected to have been in contact with a compound capable of inducing an endocrine disease or disorder may be also a subject to be diagnosed for selecting a suitable therapy. Preferably, a compound capable of inducing an endocrine disease or disorder as used herein is 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, or Ziprasidone hydrochloride.

Preferably, the at least one biomarker to be determined by the methods of the present invention is selected from any one of Tables 1a, 1 b, 4a, 4b, 6a, 6b, 7, 8a, 8b, 12a, 12b, 13a, 13b, 14a, 15a, 15b, 17a or 17b if the subject is a female.

Preferably, the at least one biomarker to be determined by the methods of the present invention is selected from any one of Tables 2a, 2b, 3a, 3b, 4c, 4d, 5a, 5b, 9, 10a, 10b, 11a, 11 b, 12c, 12d, 14b, 15c, 15d, or 16 if the subject is a male.

The term “determining the amount” as used herein refers to determining at least one characteristic feature of the biomarker, i.e. the metabolite or analyte. Characteristic features in accordance with the present invention are features which characterize the physical and/or chemical properties including biochemical properties of a biomarker. Such properties include, e.g., molecular weight, viscosity, density, electrical charge, spin, optical activity, colour, fluorescence, chemoluminescence, elementary composition, chemical structure, capability to react with other compounds, capability to elicit a response in a biological read out system (e.g., induction of a reporter gene) and the like. Values for said properties may serve as characteristic features and can be determined by techniques well known in the art. Moreover, the characteristic feature may be any feature which is derived from the values of the physical and/or chemical properties of a biomarker by standard operations, e.g., mathematical calculations such as multiplication, division or logarithmic calculus. Most preferably, the at least one characteristic feature allows the determination and/or chemical identification of the biomarker and its amount. Accordingly, the characteristic value, preferably, also comprises information relating to the abundance of the biomarker from which the characteristic value is derived. For example, a characteristic value of a biomarker may be a peak in a mass spectrum. Such a peak contains characteristic information of the biomarker, i.e. the m/z (mass to charge ratio) information, as well as an intensity value being related to the abundance of the said biomarker (i.e. its amount) in the sample.

As discussed before, the at least one biomarker to be determined in accordance with the methods of the present invention may be, preferably, determined quantitatively or semi-quantitatively. For quantitative determination, either the absolute or precise amount of the biomarker will be determined or the relative amount of the biomarker will be determined based on the value determined for the characteristic feature(s) referred to herein above. The relative amount may be determined in a case were the precise amount of a biomarker can or shall not be determined. In said case, it can be determined whether the amount in which the biomarker is present is enlarged or diminished with respect to a second sample comprising said biomarker in a second amount. Quantitatively analysing a biomarker, thus, also includes what is sometimes referred to as semi-quantitative analysis of a biomarker.

Moreover, determining as used in the methods of the present invention, preferably, includes using a compound separation step prior to the analysis step referred to before. Preferably, said compound separation step yields a time resolved separation of the at least one biomarker comprised by the sample. Suitable techniques for separation to be used preferably in accordance with the present invention, therefore, include all chromatographic separation techniques such as liquid chromatography (LC), high performance liquid chromatography (HPLC), gas chromatography (GC), thin layer chromatography, size exclusion or affinity chromatography. These techniques are well known in the art and can be applied by the person skilled in the art without further ado. Most preferably, LC and/or GC are chromatographic techniques to be envisaged by the methods of the present invention. Suitable devices for such determination of biomarkers are well known in the art. Preferably, mass spectrometry is used in particular gas chromatography mass spectrometry (GC-MS), liquid chromatography mass spectrometry (LC-MS), direct infusion mass spectrometry or Fourier transform ion-cyclotrone-resonance mass spectrometry (FT-ICR-MS), capillary electrophoresis mass spectrometry (CE-MS), high-performance liquid chromatography coupled mass spectrometry (HPLC-MS), quadrupole mass spectrometry, any sequentially coupled mass spectrometry, such as MS-MS or MS-MS-MS, inductively coupled plasma mass spectrometry (ICP-MS), pyrolysis mass spectrometry (Py-MS), ion mobility mass spectrometry or time of flight mass spectrometry (TOF). Most preferably, LC-MS and/or GC-MS are used as described in detail below. Said techniques are disclosed in, e.g., Nissen 1995, Journal of Chromatography A, 703: 37-57, U.S. Pat. No. 4,540,884 or U.S. Pat. No. 5,397,894, the disclosure content of which is hereby incorporated by reference. As an alternative or in addition to mass spectrometry techniques, the following techniques may be used for compound determination: nuclear magnetic resonance (NMR), magnetic resonance imaging (MRI), Fourier transform infrared analysis (FT-IR), ultraviolet (UV) spectroscopy, refraction index (RI), fluorescent detection, radiochemical detection, electrochemical detection, light scattering (LS), dispersive Raman spectroscopy or flame ionisation detection (FID). These techniques are well known to the person skilled in the art and can be applied without further ado. The method of the present invention shall be, preferably, assisted by automation. For example, sample processing or pre-treatment can be automated by robotics. Data processing and comparison is, preferably, assisted by suitable computer programs and databases. Automation as described herein before allows using the method of the present invention in high-throughput approaches.

Moreover, the biomarker can also be determined by a specific chemical or biological assay. Said assay shall comprise means which allow for specifically detecting the biomarker in the sample. Preferably, said means are capable of specifically recognizing the chemical structure of the biomarker or are capable of specifically identifying the biomarker based on its capability to react with other compounds or its capability to elicit a response in a biological read out system (e.g., induction of a reporter gene). Means which are capable of specifically recognizing the chemical structure of a biomarker are, preferably, detection agents which specifically bind to the biomarker, more preferably, antibodies or other proteins which specifically interact with chemical structures, such as receptors or enzymes, or aptameres. Specific antibodies, for instance, may be obtained using the biomarker as antigen by methods well known in the art. Antibodies as referred to herein include both polyclonal and monoclonal antibodies, as well as fragments thereof, such as Fv, Fab and F(ab)₂ fragments that are capable of binding the antigen or hapten. The present invention also includes humanized hybrid antibodies wherein amino acid sequences of a non-human donor antibody exhibiting a desired antigen-specificity are combined with sequences of a human acceptor antibody. Moreover, encompassed are single chain antibodies. The donor sequences will usually include at least the antigen-binding amino acid residues of the donor but may comprise other structurally and/or functionally relevant amino acid residues of the donor antibody as well. Such hybrids can be prepared by several methods well known in the art. Suitable proteins which are capable of specifically recognizing the metabolite are, preferably, enzymes which are involved in the metabolic conversion of the said biomarker. Said enzymes may either use the biomarker, e.g., a metabolite, as a substrate or may convert a substrate into the biomarker, e.g., metabolite. Moreover, said antibodies may be used as a basis to generate oligopeptides which specifically recognize the biomarker. These oligopeptides shall, for example, comprise the enzyme's binding domains or pockets for the said biomarker. Suitable antibody and/or enzyme based assays may be RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA) or solid phase immune tests. Aptameres which specifically bind to the biomarker can be generated by methods well known in the art (Ellington 1990, Nature 346:818-822; Vater 2003, Curr Opin Drug Discov Devel 6(2): 253-261). Moreover, the biomarker may also be identified based on its capability to react with other compounds, i.e. by a specific chemical reaction. Further, the biomarker may be determined in a sample due to its capability to elicit a response in a biological read out system. The biological response shall be detected as read out indicating the presence and/or the amount of the metabolite comprised by the sample. The biological response may be, e.g., the induction of gene expression or a phenotypic response of a cell or an organism.

The term “reference” refers to values of characteristic features of the at least one biomarker and, preferably, values indicative for an amount of the said biomarker which can be correlated to an endocrine disease or disorder.

Such references are, preferably, obtained from a sample derived from a subject or group of subjects which suffer from an endocrine disease or disorder or from a sample derived from a subject or group of subjects which have/has been brought into contact with 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, or Ziprasidone hydrochloride. A subject or group of subjects may be brought into contact with the said compounds by each topic or systemic administration mode as long as the compounds become bioavailable.

Preferably, the aforementioned compounds can be administered to the subject or the indiividuals of the group of subjects from which the reference is derived as described in the accompanying Exampies and Tables below.

In particular, ACTH, Cabergoline, Cyproteron Acetate, Dexamethasone, Epoxiconazole, Fenarimol, Formestane, Methimazole, Mifepristone, Risperidone, Triticonazole, or Vinclozolin shall be capable of inducing toxicity of the adrenal cortex. Moreover, 17-alpha-Ethynylestradiol, 17-alpha-Methyltestosterone, Bromocriptine Mesylate, Buserelin acetate s.c., Cabergoline, Cyproteron Acetate, Diethanolamine, Diethylstilboestrol dipropionate, Fluoxetine hydrochloride, Flutamide, Genistein, Ifosfamide, Iprodione, Leuprolide acetate salt, Medroxyprogesterone acetat, Metazachlor, Methimazole, Mibolerone, Naphthylisothiocyanate, Norethindrone acetate, Raloxifene Hydrochloride, Tamoxifen, Trenbolone, Triticonazole, or Vinclozolin shall be capable of inducing a disease or disorder related to impaired sex hormone homeostatsis. Clofibrate, Fluoroglycofen-ethyl, Glipizide, Pioglitazone hydrochloride, Rosiglitazone maleate, Streptozotocin, or Ziprasidone hydrochloride shall be, preferably, capable of inducing a disease or disorder of the endocrine pancreas.

Alternatively, but nevertheless also preferred, the reference may be obtained from sample derived from a subject or group of subjects which has not been brought into contact with 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, or Ziprasidone hydrochloride or a healthy subject or group of such subjects with respect to an endocrine disease or disorder and, more preferably, other diseases as well.

The reference may be determined as described hereinabove for the amounts of the biomarkers. In particular, a reference is, preferably, obtained from a sample of a group of subjects as referred to herein by determining the relative or absolute amounts of each of the at least one biomarker(s) in samples from each of the individuals of the group separately and subsequently determining a median or average value for said relative or absolute amounts or any parameter derived therefrom by using statistical techniques referred to elsewhere herein. Alternatively, the reference may be, preferably, obtained by determining the relative or absolute amount for each of the at least one biomarker in a sample from a mixture of samples of the group of subjects as referred to herein. Such a mixture, preferably, consists of portions of equal volume from samples obtained from each of the individuals of the said group.

Moreover, the reference, also preferably, could be a calculated reference, most preferably the average or median value, for the relative or absolute amount for each of the at least one biomarker derived from a population of individuals. Said population of individuals is the population from which the subject to be investigated by the method of the present invention originates. However, it is to be understood that the population of subjects to be investigated for determining a calculated reference, preferably, either consist of apparently healthy subjects (e.g. untreated) or comprise a number of apparently healthy subjects which is large enough to be statistically resistant against significant average or median changes due to the presence of the test subject(s) in the said population. The absolute or relative amounts of the at least one biomarker of said individuals of the population can be determined as specified elsewhere herein. How to calculate a suitable reference value, preferably, the average or median, is well known in the art. Other techniques for calculating a suitable reference include optimization using receiver operating characteristics (ROC) curve calculations which are also well known in the art and which can be performed for an assay system having a given specificity and sensitivity based on a given cohort of subjects without further ado. The population or group of subjects referred to before shall comprise a plurality of subjects, preferably, at least 5, 10, 50, 100, 1,000 or 10,000 subjects up to the entire population. More preferably, the group of subjects referred to in this context is a group of subjects having a size being statistically representative for a given population, i.e. a statistically representative sample. It is to be understood that the subject to be diagnosed by the methods of the present invention and the subjects of the said plurality of subjects are of the same species and, preferably, of the same gender.

More preferably, the reference will be stored in a suitable data storage medium such as a database and are, thus, also available for future diagnoses. This also allows efficiently diagnosing predisposition for an endocrine disease or disorder because suitable reference results can be identified in the database once it has been confirmed (in the future) that the subject from which the corresponding reference sample was obtained (indeed) developed an endocrine disease or disorder.

The term “comparing” refers to assessing whether the amount of the qualitative or quantitative determination of the at least one biomarker is identical to a reference or differs therefrom.

In case the reference results are obtained from a sample derived from a subject or group of subjects suffering from an endocrine disease or disorder or a subject or group of subjects which has been brought into contact with 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, or Ziprasidone hydrochloride, an endocrine disease or disorder can be diagnosed based on the degree of identity or similarity between the amounts obtained from the test sample and the aforementioned reference, i.e. based on an identical qualitative or quantitative composition with respect to the at least one biomarker. Identical amounts include those amounts which do not differ in a statistically significant manner and are, preferably, within at least the interval between 1st and 99th percentile, 5th and 95th percentile, 10th and 90th percentile, 20th and 80th percentile, 30th and 70th percentile, 40th and 60th percentile of the reference, more preferably, the 50th, 60th, 70th, 80th, 90th or 95th percentile of the reference. A reference obtained from a sample derived from a subject or group of subjects suffering from an endocrine disease or disorder or a subject or group of subjects which has been brought into contact with 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, or Ziprasidone hydrochloride, can be applied in the methods of the present invention in order to diagnose an endocrine disease or disorder or for determining whether a compound is capable of inducing an endocrine disease or disorder in a subject. In such a case, preferably, an amount of the at least one biomarker which is essentially identical to the reference will be indicative for the presence of an endocrine disease or disorder or a compound which is capable of inducing an endocrine disease or disorder, while an amount of the at least one biomarker which differs from the reference will be indicative for the absence of an endocrine disease or disorder or a compound which is not capable of inducing an endocrine disease or disorder.

Moreover, a reference obtained from a sample derived from a subject or group of subjects suffering from an endocrine disease or disorder or a subject or group of subjects which has been brought into contact with 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, or Ziprasidone hydrochloride, can be applied for identifying a substance for treating an endocrine disease or disorder. In such a case, preferably, an amount of the at least one biomarker which differs from the reference will be indicative for a substance suitable for treating an endocrine disease or disorder, while an amount of the at least one biomarker which is essentially identical to the reference will be indicative for a substance which is not capable of treating an endocrine disease or disorder.

In case the reference results are obtained from a sample of a subject or group of subjects which has not been brought into contact with 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, or Ziprasidone hydrochloride or which does not suffer from an endocrine disease or disorder, said an endocrine disease or disorder can be diagnosed based on the differences between the test amounts obtained from the test sample and the aforementioned reference, i.e. differences in the qualitative or quantitative composition with respect to the at least one biomarker.

The same applies if a calculated reference as specified above is used.

The difference may be an increase in the absolute or relative amount of the at least one biomarker (sometimes referred to as up-regulation of the biomarker; see also Examples) or a decrease in either of said amounts or the absence of a detectable amount of the biomarker (sometimes referred to as down-regulation of the biomarker; see also Examples). Preferably, the difference in the relative or absolute amount is significant, i.e. outside of the interval between 45th and 55th percentile, 40th and 60th percentile, 30th and 70th percentile, 20th and 80th percentile, 10th and 90th percentile, 5th and 95th percentile, 1st and 99th percentile of the reference.

A reference obtained from a sample derived from a subject or group of subjects which has not been brought into contact with 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, or Ziprasidone hydrochloride or which does not suffer from an endocrine disease or disorder can be applied in the methods of the present invention in order to diagnose the an endocrine disease or disorder or for determining whether a compound is capable of inducing an endocrine disease or disorder in a subject. In such a case, preferably, an amount of the at least one biomarker which differs from the reference will be indicative for the presence of an endocrine disease or disorder or a compound which is capable of inducing an endocrine disease or disorder, while an amount of the at least one biomarker which is essentially identical to the reference will be indicative for the absence of an endocrine disease or disorder or a compound which is not capable of inducing an endocrine disease or disorder. Moreover, a reference obtained from a sample derived from a subject or group of subjects which has not been brought into contact with 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, or Ziprasidone hydrochloride, or which does not suffer from an endocrine disease or disorder can be applied for identifying a substance for treating an endocrine disease or disorder. In such a case, preferably, an amount of the at least one biomarker which is essentially identical to the reference will be indicative for a substance suitable for treating an endocrine disease or disorder, while an amount of the at least one biomarker which differs from the reference will be indicative for a substance which is not suitable for treating an endocrine disease or disorder.

Preferred references are those referred to in the accompanying Tables or those which can be generated following the accompanying Examples. Moreover, relative differences, i.e. increases or decreases in the amounts for individual biomarkers, are preferably, those recited in the Tables below. Moreover, preferably, the extent of an observed difference, i.e. an increase or decrease, is preferably, an increase or decrease according to the factor indicated in the Tables, below.

Preferably, the at least one biomarker when selected from Tables 1a, 2a, 3a, 4a, 4c, 5a, 6a, 8a, 10a, 11a, 12a, 12c, 13a, 15a, 15c, or 17a is increased with respect to a reference obtained from a sample derived from a subject or group of subjects which has not been brought into contact with 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, or Ziprasidone hydrochloride or a sample obtained from a healthy subject or group of subjects as indicated in the said Tables.

Preferably, the at least one biomarker when selected from Tables 1 b, 2b, 3b, 4b, 4d, 5b, 6b, 7, 8b, 9, 10b, 11 b, 12b, 12d, 13b, 14a, 14b, 15b, 15d, 16, or 17b is decreased with respect to a reference obtained from a sample derived from a subject or group of subjects which has not been brought into contact with 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, or Ziprasidone hydrochloride or a sample obtained from a healthy subject or group of subjects as indicated in the said Tables.

The comparison is, preferably, assisted by automation. For example, a suitable computer program comprising algorithm for the comparison of two different data sets (e.g., data sets comprising the values of the characteristic feature(s)) may be used. Such computer programs and algorithm are well known in the art. Notwithstanding the above, a comparison can also be carried out manually.

The term “substance for treating an endocrine disease or disorder” refers to compounds which may directly interfere with the biological mechanisms inducing an endocrine disease or disorder referred to elsewhere in this specification Alternatively, but also preferred the compounds may interfere with the development or progression of symptoms associated with the an endocrine disease or disorder. Substances to be identified by the method of the present invention may be organic and inorganic chemicals, such as small molecules, polynucleotides, oligonucleotides including siRNA, ribozymes or micro RNA molecules, peptides, polypeptides including antibodies or other artificial or biological polymers, such as aptameres. Preferably, the substances are suitable as drugs, pro-drugs or lead substances for the development of drugs or pro-drugs. Thus, in an aspect of the invention, the method may further include a step comprising identifying and/or confirming the identified and selected substance a drug, pro-drug or drug or pro-drug candidate for further clinical development. Such clinical development may, preferably, includes pharmacological studies of the substance, toxicological determinations of the substance, animal and human drug testing, including clinical trials of all phases.

It is to be understood that if the methods of the present invention are to be used for identifying drugs for the therapy of an endocrine disease or disorder or for toxicological assessments of compounds (i.e. determining whether a compound is capable of inducing an endocrine disease or disorder), test samples of a plurality of subjects may be investigated for statistical reasons. Preferably, the metabolome within such a cohort of test subjects shall be as similar as possible in order to avoid differences which are caused, e.g., by factors other than the compound to be investigated. Subjects to be used for the said methods are, preferably, laboratory animals such as rodents and more preferably rats. It is to be understood further that the said laboratory animals shall be, preferably, sacrificed after completion of the methods of the present invention. All subjects of a cohort test and reference animals shall be kept under identical conditions to avoid any differential environmental influences. Suitable conditions and methods of providing such animals are described in detail in WO2007/014825. Said conditions are hereby incorporated by reference.

Accordingly, the methods of the invention aiming at identifying a substance for treating an endocrine disease or disorder and, in particular, impaired adrenal hormone homeostasis or function, sex hormone homeostasis or function or pancreatic hormone homeostasis or function, preferably, include additional steps. Preferably, further steps include carrying out preclinical studies with the substance in order to identify pharmacological and/or toxicological parameters thereof, such as ED50/EC50 and/or LD50/LC50 thresholds, carrying out clinical trials, e.g., for determining therapeutic efficacy and safety of the substance and the formulation of the identified substance in a pharmaceutically acceptable form.

The substance can, preferably, be formulated for topical or systemic administration. Conventionally, a drug will be administered intra-muscular or, subcutaneous. However, depending on the nature and the mode of action of a substance, it may, however, be administered by other routes as well. The substance is, preferably, formulated for administration in conventional dosage forms and prepared by combining the identified substance with standard pharmaceutical carriers according to conventional procedures. These procedures may involve mixing, granulating, and compression, or dissolving the ingredients as appropriate to the desired preparation. It will be appreciated that the form and character of the pharmaceutical acceptable carrier or diluent is dictated by the amount of active ingredient with which it is to be combined, the route of administration, and other well-known variables. A carrier must be acceptable in the sense of being compatible with the other ingredients of the formulation and being not deleterious to the recipient thereof. The pharmaceutical carrier employed may include a solid, a gel, or a liquid. Without being limiting, examples for solid carriers are lactose, terra alba, sucrose, talc, gelatin, agar, pectin, acacia, magnesium stearate, stearic acid and the like. Without being limiting, exemplary of liquid carriers are phosphate buffered saline solution, syrup, oil, water, emulsions, various types of wetting agents, and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pa. A diluent is selected so as not to affect the biological activity of the combination. Without being limiting, examples of such diluents are distilled water, physiological saline, Ringer's solutions, dextrose solution, and Hank's solution. In addition, the pharmaceutical composition or formulation may also include other carriers, adjuvants, or non-toxic, non-therapeutic, non-immunogenic stabilizers and the like. It is to be understood that the formulation of a substance as a drug takes place under GMP standardized conditions or the like in order to ensure quality, pharmaceutical security, and effectiveness.

The methods of the present invention can be, preferably, implemented by the device of the present invention. A device as used herein shall comprise at least the aforementioned units. The units of the device are operatively linked to each other. How to link the units in an operating manner will depend on the type of units included into the device. For example, where means for automatically qualitatively or quantitatively determining the at least one biomarker are applied in an analyzing unit, the data obtained by said automatically operating unit can be processed by the evaluation unit, e.g., by a computer program which runs on a computer being the data processor in order to facilitate the diagnosis. Preferably, the units are comprised by a single device in such a case. However, the analyzing unit and the evaluation unit may also be physically separate. In such a case operative linkage can be achieved via wire and wireless connections between the units which allow for data transfer. A wireless connection may use Wireless LAN (WLAN) or the internet. Wire connections may be achieved by optical and non-optical cable connections between the units. The cables used for wire connections are, preferably, suitable for high throughput data transport

A preferred analyzing unit for determining at least one biomarker comprises a detection agent, such as an antibody, protein or aptamere which specifically recognizes the at least one biomarker as specified elsewhere herein, and a zone for contacting said detection agent with the sample to be tested. The detection agent may be immobilized on the zone for contacting or may be applied to the said zone after the sample has been loaded. The analyzing unit shall be, preferably, adapted for qualitatively and/or quantitatively determine the amount of complexes of the detection agent and the at least one biomarker. It will be understood that upon binding of the detection agent to the at least one biomarker, at least one measurable physical or chemical property of either the at least one biomarker, the detection agent or both will be altered such that the said alteration can be measured by a detector, preferably, comprised in the analyzing unit. However, where analyzing units such as test stripes are used, the detector and the analyzing units may be separate components which are brought together only for the measurement. Based on the detected alteration in the at least one measurable physical or chemical property, the analyzing unit may calculate an intensity value for the at least one biomarker as specified elsewhere herein. Said intensity value can then be transferred for further processing and evaluation to the evaluation unit. Most preferably, the amount of the at least one biomarker can be determined by ELISA, EIA, or RIA based techniques using a detection agent as specified elsewhere herein. Alternatively, an analyzing unit as referred to herein, preferably, comprises means for separating biomarkers, such as chromatographic devices, and means for biomarker determination, such as spectrometry devices. Suitable devices have been described in detail above. Preferred means for compound separation to be used in the system of the present invention include chromatographic devices, more preferably devices for liquid chromatography, HPLC, and/or gas chromatography. Preferred devices for compound determination comprise mass spectrometry devices, more preferably, GC-MS, LC-MS, direct infusion mass spectrometry, FT-ICR-MS, CE-MS, HPLC-MS, quadrupole mass spectrometry, sequentially coupled mass spectrometry (including MS-MS or MS-MS-MS), ICP-MS, Py-MS or TOF. The separation and determination means are, preferably, coupled to each other. Most preferably, LC-MS and/or GC-MS is used in the analyzing unit referred to in accordance with the present invention.

The evaluation unit of the device of the present invention, preferably, comprises a data processing device or computer which is adapted to execute rules for carrying out the comparison as specified elsewhere herein. Moreover, the evaluation unit, preferably, comprises a database with stored references. A database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative for an endocrine disease or disorder (e.g. a query search). Thus, if an identical or similar data set can be identified in the data collection, the test data set will be associated with an endocrine disease or disorder. The evaluation unit may also preferably comprise or be operatively linked to a further database with recommendations for therapeutic or preventive interventions or life style adaptations based on the established diagnosis of an endocrine disease or disorder. Said further database can be, preferably, automatically searched with the diagnostic result obtained by the evaluation unit in order to identify suitable recommendations for the subject from which the test sample has been obtained in order to treat or prevent an endocrine disease or disorder.

In a preferred embodiment of the device of the present invention, said stored reference is a reference derived from a subject or a group of subjects known to suffer from an endocrine disease or disorder or a subject or group of subjects which has been brought into contact with at least one compound selected from the group consisting of 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, or Ziprasidone hydrochloride, and said data processor executes instructions for comparing the amount of the at least one biomarker determined by the analyzing unit to the stored reference, wherein an essentially identical amount of the at least one biomarker in the test sample in comparison to the reference is indicative for the presence of an endocrine disease or disorder or wherein an amount of the at least one biomarker in the test sample which differs in comparison to the reference is indicative for the absence of an endocrine disease or disorder.

In another preferred embodiment of the device of the present invention, said stored reference is a reference derived from a subject or a group of subjects known not to suffer from an endocrine disease or disorder or a subject or group of subjects which has not been brought into contact with at least one compound selected from the group consisting of 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, and Ziprasidone hydrochloride, and said data processor executes instructions for comparing the amount of the at least one biomarker determined by the analyzing unit to the stored reference, wherein an amount of the at least one biomarker in the test sample which differs in comparison to the reference is indicative for the presence of an endocrine disease or disorder or wherein an essentially identical amount of the at least one biomarker in the test sample in comparison to the reference is indicative for the absence of an endocrine disease or disorder.

The device, thus, can also be used without special medical knowledge by medicinal or laboratory staff or patients, in particular when an expert system making recommendations is included. The device is also suitable for near-patient applications since the device can be adapted to a portable format.

The term “kit” refers to a collection of the aforementioned components, preferably, provided separately or within a single container. The container also comprises instructions for carrying out the method of the present invention. These instructions may be in the form of a manual or may be provided by a computer program code which is capable of carrying out the comparisons referred to in the methods of the present invention and to establish a diagnosis accordingly when implemented on a computer or a data processing device. The computer program code may be provided on a data storage medium or device such as an optical or magnetic storage medium (e.g., a Compact Disc (CD), CD-ROM, a hard disk, optical storage media, or a diskette) or directly on a computer or data processing device. A “standard” as referred to in connection with the kit of the invention is an amount of the at least one biomarker when present in solution or dissolved in a predefined volume of a solution resembles the amount of the at least one biomarker which is present (i) in a subject or a group of subjects known to suffer from an endocrine disease or disorder or a subject or group of subjects which has been brought into contact with at least one compound selected from the group consisting of 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, and Ziprasidone hydrochloride or (ii) derived from a subject or a group of subjects known to not suffer from therefrom or a subject or group of subjects which has not been brought into contact with at least one compound selected from the group consisting of 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, and Ziprasidone hydrochloride.

Advantageously, it has been found in the study underlying the present invention that the amount of at least one biomarker as specified herein allows for diagnosing an endocrine disease or disorder, specifically an endocrine disease or disorder induced by 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, or Ziprasidone hydrochloride. The specificity and accuracy of the method will be even more improved by determining an increasing number or even all of the aforementioned biomarkers. A change in the quantitative and/or qualitative composition of the metabolome with respect to these specific biomarkers is indicative for an endocrine disease or disorder even before other signs of the said toxicity are clinically apparent. The morphological, physiological as well as biochemical parameters which are currently used for diagnosing an endocrine disease or disorder are less specific and less sensitive in comparison to the biomarker determination provided by the present invention. Thanks to the present invention, an endocrine disease or disorder of a compound can be more efficiently and reliably assessed. Moreover, based on the aforementioned findings, screening assays for drugs which are useful for the therapy of an endocrine disease or disorder are feasible.

In general, the present invention contemplates the use of at least one biomarker in a sample of a subject selected from any one of the Tables 1a, 1 b, 2a, 2b, 3a, 3b, 4a, 4b, 4c, 4d, 5a, 5b, 6a, 6b, 7, 8a, 8b, 9, 10a, 10b, 11a, 11b, 12a, 12b, 12c, 12d, 13a, 13b, 14a, 14b, 15a, 15b, 15c, 15d, 16, 17a, or 17b or a detection agent for said biomarker for diagnosing an endocrine disease or disorder, for determining whether a compound is capable of inducing an endocrine disease or disorder or for identifying a substance capable of treating an endocrine disease or disorder. Further, the present invention, in general, contemplates the use of the at least one biomarker in a sample of a subject or a detection agent therefor for identifying a subject being susceptible for a treatment of an endocrine disease or disorder. Preferred detection agents to be used in this context of the invention are those referred to elsewhere herein. Moreover, the methods of the present invention can be, advantageously, implemented into a device. Furthermore, a kit can be provided which allows for carrying out the methods.

The present invention also relates to a data collection comprising characteristic values for the biomarkers recited in any one of Tables 1a, 1 b, 2a, 2b, 3a, 3b, 4a, 4b, 4c, 4d, 5a, 5b, 6a, 6b, 7, 8a, 8b, 9, 10a, 10b, 11a, 11b, 12a, 12b, 12c, 12d, 13a, 13b, 14a, 14b, 15a, 15b, 15c, 15d, 16, 17a, or 17b. The term “data collection” refers to a collection of data which may be physically and/or logically grouped together. Accordingly, the data collection may be implemented in a single data storage medium or in physically separated data storage media being operatively linked to each other. Preferably, the data collection is implemented by means of a database. Thus, a database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative for an endocrine disease or disorder (e.g. a query search). Thus, if an identical or similar data set can be identified in the data collection, the test data set will be associated with an endocrine disease or disorder. Consequently, the information obtained from the data collection can be used to diagnose an endocrine disease or disorder based on a test data set obtained from a subject.

Moreover, the present invention pertains to a data storage medium comprising the said data collection. The term “data storage medium” as used herein encompasses data storage media which are based on single physical entities such as a CD, a CD-ROM, a hard disk, optical storage media, or a diskette. Moreover, the term further includes data storage media consisting of physically separated entities which are operatively linked to each other in a manner as to provide the aforementioned data collection, preferably, in a suitable way for a query search.

The present invention also relates to a system comprising
(a) means for comparing characteristic values of at least one biomarker of a sample operatively linked to
(b) the data storage medium of the present invention.

The term “system” as used herein relates to different means which are operatively linked to each other. Said means may be implemented in a single device or may be implemented in physically separated devices which are operatively linked to each other. The means for comparing characteristic values of the biomarker operate, preferably, based on an algorithm for comparison as mentioned before. The data storage medium, preferably, comprises the aforementioned data collection or database, wherein each of the stored data sets being indicative for an endocrine disease or disorder. Thus, the system of the present invention allows identifying whether a test data set is comprised by the data collection stored in the data storage medium. Consequently, the system of the present invention may be applied as a diagnostic means in diagnosing an endocrine disease or disorder. In a preferred embodiment of the system, means for determining characteristic values of biomakers of a sample are comprised. The term “means for determining characteristic values of biomarkers” preferably relates to the aforementioned devices for the determination of biomarkers such as mass spectrometry devices, ELISA devices, NMR devices or devices for carrying out chemical or biological assays for the analytes.

All references referred to above are herewith incorporated by reference with respect to their entire disclosure content as well as their specific disclosure content explicitly referred to in the above description.

The following Examples are merely for the purposes of illustrating the present invention. They shall not be construed, whatsoever, to limit the scope of the invention in any respect.

### EXAMPLES

### Example

### Biomarkers Associated with an Endocrine Disease or Disorder

A group of each 5 male and female rats was dosed once daily with the indicated compounds (see Table 10, below for compounds, applied doses and administration details) over 28 days.

Each dose group in the studies consisted of five rats per sex. Additional groups of each 5 male and female animals served as controls. Before starting the treatment period, animals, which were 62-64 days old when supplied, were acclimatized to the housing and environmental conditions for 7 days. All animals of the animal population were kept under the same constant temperature (20-24±3° C.) and the same constant humidity (30-70%). The animals of the animal population were fed ad libitum. The food to be used was essentially free of chemical or microbial contaminants. Drinking water was also offered ad libitum. Accordingly, the water was free of chemical and microbial contaminants as laid down in the European Drinking Water Directive 98/83/EG. The illumination period was 12 hours light followed by 12 hours darkness (12 hours light, from 6:00 to 18:00, and 12 hours darkness, from 18:00 to 6:00). The studies were performed in an AAALAC-approved laboratory in accordance with the German Animal Welfare Act and the European Council Directive 86/609/EE. The test system was arranged according to the OECD 407 guideline for the testing of chemicals for repeated dose 28-day oral toxicity study in rodents. The test substances (compounds) in the Tables 1 to 17 below were dosed and administered as described in the Table 18.

In the morning of day 7, 14, and 28, blood was taken from the retroorbital venous plexus from fasted anaesthetized animals. From each animal, 1 ml of blood was collected with EDTA as anticoagulant. The samples were centrifuged for generation of plasma. All plasma samples were covered with a N2 atmosphere and then stored at −80° C. until analysis.

For mass spectrometry-based metabolite profiling analyses plasma samples were extracted and a polar and a non-polar (lipid) fraction was obtained. For GC-MS analysis, the non-polar fraction was treated with methanol under acidic conditions to yield the fatty acid methyl esters. Both fractions were further derivatised with O-methyl-hydroxyamine hydrochloride and pyridine to convert Oxogroups to O-methyloximes and subsequently with a silylating agent before analysis. In LC-MS analysis, both fractions were reconstituted in appropriate solvent mixtures. HPLC was performed by gradient elution on reversed phase separation columns. Mass spectrometric detection which allows target and high sensitivity MRM (Multiple Reaction Monitoring) profiling in parallel to a full screen analysis was applied as described in WO2003073464.

Steroids and their metabolites were measured by online SPE-LC-MS (Solid phase extraction-LC-MS). Catecholamines and their metabolites were measured by online SPE-LC-MS as described by Yamada et al. (Yamada 2002, Journal of Analytical Toxicology, 26(1): 17-22))

Following comprehensive analytical validation steps, the data for each analyte were normalized against data from pool samples. These samples were run in parallel through the whole process to account for process variability. The significance of treatment group values specific for sex, treatmentduration and metabolite was determined by comparing means of the treated groups to the means of the respective untreated control groups using WELCH-test and quantified with treatment ratios versus control and p-values.

The identification of the most important biomarkers per toxicity pattern was done by a ranking of the analytes in the tables below. Therefore the metabolic changes in reference treatments of a given pattern (shown in the table) were compared with changes of the same metabolite in other unrelated treatments. For each metabolite T-values were obtained for the reference and control treatment and compared by the Welch test to assess whether these two groups are significantly different. The maximum absolute value of the respective TVALUE was taken to indicate the most important metabolite for the pattern.

The changes of the group of plasma metabolites being indicative for an endocrine disease or disorder after treatment of the rats are shown in the following tables:

TABLE 1a
Markers for Adrenal antiglucocorticoid in female rats; Significant
up-regulation changes (p-Value ≦ 0.2) are marked (*). For
some metabolites (marked with #), additional information are
provided in table 19. All compounds were administered in high dose.

| | Cabergoline | | | Mifepristone | | |
|---|---|---|---|---|---|---|
| Metabolite | f7 | f14 | f28 | f7 | f14 | f28 |
| Cytosine | 1.22 * | 1.33 * | 1.3 * | 1.11 | 1.16 * | 1.18 * |
| 3-Hydroxy- | 1.31 * | 1.32 * | 1.01 | 1.21 * | 1.18 * | 1.34 * |
| butyrate | | | | | | |

TABLE 1b
Markers for Adrenal antiglucocorticoid in female rats; Significant down-regulation changes
(p-Value ≦ 0.2) are marked (*). For some metabolites (marked with #), additional
information are provided in table 19. All compounds were administered in high dose.

| | Cabergoline | | | Mifepristone | | |
|---|---|---|---|---|---|---|
| Metabolite | f7 | f14 | f28 | f7 | f14 | f28 |
| Citrulline | 0.75 * | 0.93 * | 0.81 | 0.89 * | 0.89 * | 0.88 * |
| dihomo-gamma-Linolenic acid | 0.4 * | 0.58 * | 0.26 * | 0.68 * | 0.51 * | 0.74 |
| (C20:cis[8,11,14]3) | | | | | | |
| Uric acid | 0.67 * | 0.71 * | 0.65 * | 0.87 | 0.73 * | 0.74 * |
| Ceramide (d18:1, C24:0) | 0.53 * | 0.57 * | 0.36 * | 0.47 * | 0.64 * | 0.54 * |
| Ascorbic acid | 0.83 | 0.71 * | 0.96 * | 0.93 * | 0.8 * | 0.94 * |
| Eicosaenoic acid (C20:1) No 02# | 0.4 * | 0.32 * | 0.25 * | 0.48 * | 0.35 * | 1.12 |
| Citrate | 0.89 * | 0.91 * | 0.8 * | 0.83 * | 0.94 | 0.89 * |
| Choline plasmalogen No 02# | 0.76 * | 0.76 * | 0.3 * | 0.99 | 0.83 * | 0.85 * |
| Homovanillic acid (HVA) | 0.58 * | 0.68 * | 0.41 * | 0.7 * | 0.93 | 0.87 * |
| Docosahexaenoic acid | 1.07 | 0.48 * | 0.21 * | 0.73 * | 0.78 * | 0.75 * |
| (C22:cis[4,7,10,13,16,19]6) | | | | | | |
| Cholic acid | 2.54 | 0.07 * | 0.01 * | 0.12 * | 0.12 | 0 * |
| Creatinine | 0.67 * | 0.87 * | 0.9 * | 0.95 | 1 | 0.96 * |
| Heptadecanoic acid (C17:0) | 0.85 | 0.61 * | 0.47 * | 0.85 * | 0.71 * | 0.73 * |
| Behenic acid (C22:0) | 0.65 * | 0.56 * | 0.17 * | 0.71 * | 0.71 * | 1 |
| alpha-Tocopherol | 0.81 * | 0.56 * | 0.35 * | 0.75 | 0.76 * | 0.61 * |
| Glycochenodeoxycholic acid | 1.23 | 0.32 * | 0.74 * | 0.15 * | 2.07 | 0.17 * |
| Phosphatidylcholine No 04# | 0.75 * | 0.53 * | 0.34 * | 0.67 * | 0.8 * | 0.69 * |
| Progesterone | 0.17 * | 0.43 * | 0.2 * | 0.67 | 0.45 * | 0.38 * |
| Corticosterone | 0.28 * | 0.09 * | 0.68 * | 0.52 | 0.54 * | 0.53 * |
| Elaidic acid (C18:trans[9]1) | 0.66 * | 0.5 * | 0.33 * | 0.74 * | 0.71 * | 0.79 |
| Phosphate, lipid fraction | 0.73 * | 0.57 * | 0.36 * | 0.68 * | 0.84 * | 0.94 |
| Hippuric acid | 0.45 * | 0.71 * | 0.31 * | 0.49 * | 0.63 * | 1.18 |
| Stearic acid (C18:0) | 0.79 * | 0.42 * | 0.28 * | 0.61 * | 0.81 * | 0.8 * |
| myo-Inositol, lipid fraction | 0.87 * | 0.48 * | 0.23 * | 0.63 * | 0.77 * | 0.86 |
| Lignoceric acid (C24:0) | 0.54 * | 0.33 * | 0.15 * | 0.55 * | 0.62 * | 0.74 * |
| Cholesterol | 0.72 * | 0.36 * | 0.16 * | 0.65 * | 0.72 * | 0.76 |
| Arachidonic acid | 0.71 * | 0.32 * | 0.16 * | 0.64 * | 0.71 * | 0.78 |
| (C20:cis[5,8,11,14]4) | | | | | | |
| Isopalmitic acid (C16:0) | 0.59 * | 0.54 * | 0.36 * | 0.85 | 0.72 * | 0.65 * |
| Campesterol | 0.54 * | 0.3 * | 0.1 * | 0.61 * | 0.66 * | 0.83 |
| Choline plasmalogen No 03# | 0.81 * | 0.63 * | 0.39 * | 0.89 * | 0.77 * | 0.74 * |
| Lysophosphatidylcholine (C17:0)# | 0.83 * | 0.72 * | 0.57 * | 0.98 | 0.95 * | 0.89 * |
| Phosphatidylcholine (C18:0, C18:1)# | 0.87 * | 0.85 | 0.58 * | 0.85 | 0.8 * | 0.7 * |
| Galactose, lipid fraction | 0.79 | 0.48 * | 0.24 * | 0.63 * | 0.76 * | 0.92 |
| Alanine | 1.21 | 0.84 * | 0.89 * | 0.82 * | 0.75 * | 0.82 * |
| Glycerol, polar fraction | 0.7 * | 0.97 | 0.68 * | 0.9 | 1.01 | 0.88 * |

TABLE 2a
Markers for Adrenal Cortex Steroid synthese inhibition in male rats; Significant up-regulation
changes (p-Value ≦ 0.2) are marked (*). For some metabolites (marked with #), additional
information are provided in table 19. All compounds were administered in high dose.

| | Dexamethasone | | | Epoxiconazole | | | Cyproteron Acetate | | |
|---|---|---|---|---|---|---|---|---|---|
| Metabolite | m7 | m14 | m28 | m7 | m14 | m28 | m7 | m14 | m28 |
| Tricosanoic acid (C23:0) | 1.28 | 1.7 * | 1.86 * | 1.45 * | 2.1 * | 1.68 * | 1.48 * | 1.3 * | 1.39 * |
| Ceramide (d18:1, C24:1) | 1.62 | 1.99 * | 2.77 * | 1.69 * | 3.28 * | 2.21 * | 1 | 1.47 * | 2.22 * |
| alpha-Tocopherol | 0.93 | 1.53 * | 1.43 * | 1.23 | 1.37 * | 1.48 * | 1.22 * | 1.32 * | 1.44 * |

TABLE 2b
Markers for Adrenal Cortex Steroid synthese inhibition in male rats; Significant down-regulation
changes (p-Value ≦ 0.2) are marked (*). For some metabolites (marked with #), additional
information are provided in table 19. All compounds were administered in high dose.

| | | | | | | | Cyproteron | | |
|---|---|---|---|---|---|---|---|---|---|
| | Dexamethasone | | | Epoxiconazole | | | Acetate | | |
| Metabolite | m7 | m14 | m28 | m7 | m14 | m28 | m7 | m14 | m28 |
| trans-4-Hydroxyproline | 0.69 * | 0.45 * | 0.31 * | 0.77 * | 0.83 * | 0.88 * | 0.53 * | 0.44 * | 0.52 * |
| 18-Hydroxy-11- | 0.11 * | 0.07 * | 0.02 * | 0.31 * | 0.4* | 0.93 | 0.08 * | 0.08 * | 0.02 * |
| deoxycorticosterone | | | | | | | | | |
| 3-Hydroxybutyrate | 0.27 * | 0.25 * | 0.24 * | 0.62 * | 0.65 * | 0.71 * | 0.58 * | 0.73 * | 0.68 * |
| Coenzyme Q10 | 0.59 * | 0.67 * | 1.12 | 0.36 * | 0.24 * | 0.23 * | 0.49 * | 0.86 | 0.7 * |
| Corticosterone | 0.09 * | 0.03 * | 0 * | 0.31 | 0.39 * | 1.25 | 0.04 * | 0.01 * | 0 * |
| Metanephrine | 0.99 | 0.64 * | 0.68 * | 0.62 * | 0.53 * | 0.81 * | 0.91 | 0.66 * | 0.58 * |

TABLE 3a
Markers for Adrenals aromatase inhibitor in male rats; Significant up-regulation changes
(p-Value ≦ 0.2) are marked (*). For some metabolites (marked with #), additional
information are provided in table 19. All compounds were administered in low dose.

| | Formestane | | | Epoxiconazole | | | Fenarimol | | | Triticonazole | | |
|---|---|---|---|---|---|---|---|---|---|---|---|---|
| Metabolite | m7 | m14 | m28 | m7 | m14 | m28 | m7 | m14 | m28 | m7 | m14 | m28 |
| Threonic | 1.27 * | 1.24 * | 1.44 * | 1.11 * | 1.17 * | 1.39 * | 1.37 * | 1.3 * | 1.34 * | 1.28 * | 1.43 * | 1.79 * |
| acid | | | | | | | | | | | | |

TABLE 3b
Markers for Adrenals aromatase inhibitor in male rats; Significant down-regulation changes
(p-Value ≦ 0.2) are marked (*). For some metabolites (marked with #), additional information
are provided in table 19. All compounds were administered in low dose.

| | Formestane | | | Epoxiconazole | | | Fenarimol | | | Triticonazole | | |
|---|---|---|---|---|---|---|---|---|---|---|---|---|
| Metabolite | m7 | m14 | m28 | m7 | m14 | m28 | m7 | m14 | m28 | m7 | m14 | m28 |
| Lysophosphatidylcholine | 0.99 * | 0.97 * | 0.96 * | 0.96 * | 0.9 * | 0.98 | 0.99 | 0.96 * | 0.97 * | 0.96 * | 0.92 * | 0.91 * |
| (C20:4)# | | | | | | | | | | | | |
| Glucose | 0.8 * | 0.89 * | 0.9 * | 0.78 * | 0.72 * | 0.88 * | 0.9 | 0.9 * | 0.91 * | 0.82 * | 0.8 * | 1 |

TABLE 4a
Markers for Adrenals (Hypocortisolism) in female rats;
Significant up-regulation changes (p-Value ≦ 0.2) are marked (*). For some
metabolites (marked with #), additional information are provided in table 19.
All compounds were administered in high dose.

| | Dexamethasone | ACTH |
|---|---|---|
| Metabolite | f28 | f28 |
| 5-Oxoproline | 1.19 * | 1.05 * |
| Mannose | 1.2 * | 1.04 * |
| Lysophosphatidylcholine | 1.33 * | 1.06 * |
| (C18:2)# | | |
| Tyrosine | 1.69 * | 1.26 * |
| TAG No 059# | 2.19 * | 1.62 * |
| Lysophosphatidylcholine | 1.28 * | 1.11 * |
| (C20:4)# | | |

TABLE 4b
Markers for Adrenals (Hypocortisolism) in female rats; Significant
down-regulation changes (p-Value ≦ 0.2) are marked (*). For some
metabolites (marked with #), additional information are provided in
table 19. All compounds were administered in high dose.

| | Dexamethasone | ACTH |
|---|---|---|
| Metabolite | f28 | f28 |
| Choline plasmalogen No 03# | 0.83 * | 0.77 * |
| Biotin | 0.65 * | 0.7 * |
| alpha-Tocopherol | 0.85 * | 0.95 * |
| Citrate | 0.8 * | 0.92 * |
| Ketoleucine | 0.8 * | 0.76 * |
| Malate | 0.61 * | 0.76 * |
| 21-Hydroxyprogesterone (11- | 0.04 * | 0.31 * |
| Deoxycorticosterone) | | |
| Lactate | 0.65 * | 0.68 * |
| Corticosterone | 0 * | 0.67 * |
| 18-Hydroxy-11-deoxycorticosterone | 0 * | 0.69 * |
| 3-Hydroxybutyrate | 0.18 * | 0.85 * |

TABLE 4c
Markers for Adrenals (Hypocortisolism) in male rats; Significant
up-regulation changes (p-Value ≦ 0.2) are marked (*). For some metabolites
(marked with #), additional information are provided in table 19.
All compounds were administered in high dose.

| | ACTH | Dexamethasone |
|---|---|---|
| Metabolite | m28 | m28 |
| Lysophosphatidylethanolamine | 1.22 * | 1.35 * |
| (C22:5)# | | |
| Urea | 1.28 * | 1.13 |

TABLE 4d
Markers for Adrenals (Hypocortisolism) in male rats; Significant
down-regulation changes (p-Value ≦ 0.2) are marked (*). For some metabolites
(marked with #), additional information are provided in table 19.
All compounds were administered in high dose.

| | ACTH | Dexamethasone |
|---|---|---|
| Metabolite | m28 | m28 |
| 3,4-Dihydroxyphenylalanine (DOPA) | 0.86 * | 0.77 * |
| Phosphatidylcholine No 04# | 0.8 * | 0.89 * |
| Biotin | 0.64 * | 0.56 * |
| 21-Hydroxyprogesterone (11- | | |
| Deoxycorticosterone) | 0.18 * | 0.06 * |
| Ketoleucine | 0.81 * | 0.87 * |
| Citrate | 0.82 * | 0.36 * |
| Progesterone | 0.14 * | 0.05 * |
| 18-Hydroxy-11-deoxycorticosterone | 0.43 * | 0 * |
| Corticosterone | 0.42 * | 0 * |
| 3-Hydroxybutyrate | 0.65 * | 0.19 * |

TABLE 5a
Markers for adrenals cortex synth. inhibitors in male rats; Significant up-regulation
changes (p-Value ≦ 0.2) are marked (*). For some metabolites (marked with #), additional
information are provided in table 19. All compounds were administered in high dose.

| | Methimazole | | | Vinclozolin | | | Epoxiconazole | | |
|---|---|---|---|---|---|---|---|---|---|
| Metabolite | m7 | m14 | m28 | m7 | m14 | m28 | m7 | m14 | m28 |
| 3-O-Methylsphingosine | 3.59 * | 3.98 * | 4.59 * | 1.63 * | 1.39 * | 1.9 * | 2.4 * | 3.66 * | 3.22 * |
| (d18:1)# | | | | | | | | | |
| threo-Sphingosine | 2.91 * | 3.72 * | 4.28 * | 1.63 * | 1.53 * | 1.34 * | 2.24 * | 2.98 * | 2.77 * |
| (d18:1) | | | | | | | | | |
| erythro-Sphingosine | 2.91 * | 3.71 * | 3.44 * | 1.51 * | 1.37 * | 1.61 * | 2.15 * | 2.94 * | 2.8 * |
| (d18:1) | | | | | | | | | |
| 5-O-Methylsphingosine | 3.41 * | 4.75 * | 4.37 * | 1.65 * | 1.51 * | 1.72 * | 2.84 * | 3.5 * | 3.34 * |
| (d18:1)# | | | | | | | | | |
| Nervonic acid | 2.59 * | 3 * | 2.23 * | 1.74 * | 1.93 * | 1.63 * | 2.65 * | 3.96 * | 3.59 * |
| (C24:cis[15]1) | | | | | | | | | |
| Docosapentaenoic acid | 1.16 | 1.17 | 1.24 * | 1.32 * | 1.28 * | 1.14 | 1.53 * | 1.89 * | 1.74 * |
| (C22:cis[7, 10, 13, 16, 19]5) | | | | | | | | | |
| Lignoceric acid (C24:0) | 2.12 * | 2.82 * | 2.16 * | 1.45 * | 1.25 * | 1.26 * | 2.01 * | 2.38 * | 2.29 * |
| Choline plasmalogen | 1.35 * | 1.45 * | 1.23 * | 1.1 | 1.23 * | 1.24 * | 1.29 * | 1.05 | 1.09 |
| (C18, C20:4) | | | | | | | | | |
| Ceramide (d18:1, C24:1) | 1.88 * | 2.41 * | 1.89 * | 1.19 * | 1.45 * | 1.37 * | 1.69 * | 3.28 * | 2.21 * |
| Sphingomyelin | 1.35 * | 1.64 * | 1.41 * | 1.12 | 1.18 * | 1.24 * | 1.63 * | 1.89 * | 1.53 * |
| (d18:1, C24:0)# | | | | | | | | | |
| Ceramide (d18:1, C24:0) | 2.58 * | 2.83 * | 2.72 * | 1.23 * | 1.44 * | 1.21 * | 1.42 * | 2.26 * | 1.75 * |
| Phosphatidylcholine | 1.17 * | 0.97 | 1.22 * | 1.1 * | 1.46 * | 1.17 * | 1.47 * | 1.81 * | 1.74 * |
| (C18:0, C22:6)# | | | | | | | | | |
| alpha-Tocopherol | 2.26 * | 3.65 * | 2.04 * | 1.28 * | 1.14 * | 1.33 * | 1.23 | 1.37 * | 1.48 * |
| Phytosphingosine | 2 * | 3.12 * | 3.11 * | 1.28 * | 1.33 * | 1.22 * | 1.76 * | 2.06 * | 1.8 * |
| Behenic acid (C22:0) | 2.19 * | 3.12 * | 3.13 * | 1.26 * | 1.31 * | 1.29 | 1.68 * | 2.26 * | 1.89 * |
| Tricosanoic acid (C23:0) | 1.93 * | 2.69 * | 2.5 * | 1.29 * | 1.14 * | 1.21 * | 1.45 * | 2.1 * | 1.68* |
| 1,5-Anhydrosorbitol | 0.81 * | 0.98 | 1.06 | 1.18 * | 1.25 * | 1.61 * | 1.24 * | 1.27 * | 1.19 * |
| Threonic acid | 0.72 * | 0.69 * | 0.69 * | 1.51 * | 1.95 * | 2.14 * | 1.56 * | 1.32 * | 1.68 * |
| Sphingomyelin | 2.24 * | 2.27 * | 1.92 * | 1.17 | 1.29 * | 1.34 * | 1.79 * | 1.72 * | 1.93 * |
| (d18:1, C16:0) | | | | | | | | | |
| Isoleucine | 0.98 | 1.18 * | 1.38 * | 1.07 * | 1.33 * | 1.15 * | 1.04 * | 1.05 | 1.09 * |

TABLE 5b
Markers for adrenals cortex synth. inhibitors in male rats; Significant down-regulation
changes (p-Value ≦ 0.2) are marked (*). For some metabolites (marked with #), additional
information are provided in table 19. All compounds were administered in high dose.

| | Methimazole | | | Vinclozolin | | | Epoxiconazole | | |
|---|---|---|---|---|---|---|---|---|---|
| Metabolite | m7 | m14 | m28 | m7 | m14 | m28 | m7 | m14 | m28 |
| Linolenic acid | 0.7 * | 0.8 * | 0.7 * | 0.99 | 0.67 | 0.64 * | 0.73 * | 0.59 * | 0.43 * |
| (C18:cis[9, 12, 15]3) | | | | | | | | | |
| trans-4-Hydroxyproline | 0.85 * | 0.76 * | 0.92 * | 0.95 | 0.79 * | 0.85 * | 0.77 * | 0.83 * | 0.88 * |
| 18-Hydroxy-11- | 0.38 * | 0.82 | 0.24 * | 0.27 * | 0.29 * | 0.51 * | 0.31 * | 0.4 * | 0.93 |
| deoxycorticosterone | | | | | | | | | |
| Glycerol, polar fraction | 0.74 * | 0.64 * | 0.71 * | 0.78 * | 0.84 * | 0.77 * | 0.81 * | 0.87 * | 0.91 |
| Glucose | 1.02 | 1.11 | 1.06 * | 0.97 | 0.96 * | 0.83 * | 0.72 * | 0.79 * | 0.71 * |
| Corticosterone | 0.38 * | 1.47 * | 0.63 | 0.25 * | 0.14 * | 0.58 * | 0.31 | 0.39 * | 1.25 |
| Coenzyme Q9 | 1.23 | 1.54 * | 2.14 * | 0.82 | 0.86 * | 0.79 | 0.64 * | 0.66 * | 0.52 * |

TABLE 6a
Markers for Adrenals Steroid synth. Inhibitors in female rats; Significant up-regulation
changes (p-Value ≦ 0.2) are marked (*). For some metabolites (marked with #),
additional information are provided in table 19. All compounds were administered in high dose.

| | Epoxiconazole | | | Risperidone | | | Dexamethasone | | |
|---|---|---|---|---|---|---|---|---|---|
| Metabolite | f7 | f14 | f28 | f7 | f14 | f28 | f7 | f14 | f28 |
| TAG (C18:2, C18:3)# | 2.23 * | 2.56 * | 2.58 * | 5.67 * | 4.97 * | 2.97 * | 2.57 * | 1.74 * | 3.28 * |
| TAG | 3.01 * | 3.04 * | 2.97 * | 2.3 * | 3.51 * | 1.79 * | 3.22 * | 1.89 * | 2.9 * |
| (C18:3, C18:2, C18:1)# | | | | | | | | | |
| Linoleic acid | 1.78 * | 1.83 * | 1.62 * | 1.87 * | 1.67 * | 1.46 * | 1.66 * | 1.27 * | 1.26 * |
| (C18:cis[9,12]2) | | | | | | | | | |
| Glycerol, lipid fraction | 1.7 * | 1.95 * | 1.73 * | 2.2 * | 2.51 * | 1.93 * | 1.8 * | 1.32 * | 2.1 * |
| TAG (C18:1, C18:2)# | 2.75 * | 3.52 * | 1.92 * | 3.33 * | 4.13 * | 2.04 * | 2.29 * | 1.39 * | 2.12 * |
| TAG (C16:0, C18:2)# | 2.98 * | 3.29 * | 1.91 * | 3 * | 3.04 * | 2.23 * | 2.47 * | 1.8 * | 2.17 * |
| TAG (C18:2, C18:2)# | 2.67 * | 3.85 * | 2.16 * | 4.31 * | 4.14 * | 2.33 * | 2.71 * | 1.87 * | 2.87 * |
| Docosapentaenoic acid | 1.89 * | 2.47 * | 1.22 * | 4.1 * | 3.68 * | 2.78 * | 1.46 * | 1.55 * | 2.3 * |
| (C22:cis[7,10,13,16,19]5) | | | | | | | | | |
| TAG No 01 # | 1.7 * | 2.34 * | 2.54 * | 3.52 * | 5.09 * | 3.64 * | 2.95 * | 1.54 * | 2.56 * |
| Cholesterolester No 01# | 1.46 * | 1.49 * | 1.47 * | 1.1 * | 1.09 * | 1.07 * | 1.04 * | 0.94 | 1.1 * |
| Lysophosphatidylcholine | 1.36 * | 1.34 * | 1.21 * | 1.3 * | 1.23 * | 1.16 * | 1.15 * | 1.13 * | 1.18 * |
| (C18:1)# | | | | | | | | | |
| DAG (C18:1, C18:2)# | 1.55 * | 1.89 * | 1.61 * | 2.05 * | 2.29 * | 2.09 * | 1.64 * | 1.45 * | 1.69 * |
| TAG No 07# | 1.81 * | 2.4 * | 1.73 * | 5.76 * | 6.65 * | 3.47 * | 2.69 * | 1.91 * | 2.9 * |
| TAG | 2.29 * | 2.25 * | 2.3 * | 5.42 * | 3.14 * | 2.91 * | 3.09 * | 2.31 * | 2.83 * |
| (C16:0, C18:1, C18:3)# | | | | | | | | | |
| Glycerol-3-phosphate, | 1.84 * | 1.84 * | 1.59 * | 1.43 * | 1.32 * | 1.06 * | 1.25 * | 1.8 * | 1.54 * |
| polar fraction | | | | | | | | | |
| Palmitic acid (C16:0) | 1.58 * | 1.8 * | 1.34 * | 1.94 * | 1.95 * | 1.69 * | 1.52* | 1.15 * | 1.39 * |
| dihomo-gamma-Linolenic | 2.15 * | 3.63 * | 2.01 * | 2.46 * | 2.76 * | 1.7 * | 1.36 * | 1.58 * | 1.8 * |
| acid (C20:cis[8,11,14]3) | | | | | | | | | |
| TAG (C16:0, C16:1)# | 1.13 | 1.55 * | 1.61 * | 5.33 * | 9.21 * | 4.05 * | 1.95 * | 1.6 | 2.51 * |
| Phosphatidylcholine | 1.05 * | 1.15 * | 1.06 * | 1 * | 1 * | 1 * | 1.03 * | 1.01 * | 1 |
| (C16:0, C18:2)# | | | | | | | | | |
| TAG (DAG-Fragment)# | 2.8 * | 2.98 * | 2.07 * | 4.03 * | 4.45 * | 2.21 * | 3.42 * | 2.05 * | 3.46 * |
| TAG No 059# | 3.39 * | 3.79 * | 2.3 * | 1.95 * | 2.24 * | 1.41 * | 2.1 * | 1.52 * | 2.19 * |

TABLE 6b
Markers for Adrenals Steroid synth. Inhibitors in female rats; Significant down-regulation
changes (p-Value ≦ 0.2) are marked (*). For some metabolites (marked with #),
additional information are provided in table 19. All compounds were administered in high dose.

| | Epoxiconazole | | | Risperidone | | | Dexamethasone | | |
|---|---|---|---|---|---|---|---|---|---|
| Metabolite | f7 | f14 | f28 | f7 | f14 | f28 | f7 | f14 | f28 |
| TAG No 02# | 0.79 * | 0.97 * | 0.81 * | 0.71 * | 0.51 * | 0.6 * | 0.7 * | 0.55 | 0.52 * |
| 18-Hydroxy-11- | 0.45 * | 0.37 * | 0.4 * | 0.28 * | 0.55 * | 0.29 * | 0.02 * | 0.09 * | 0.01 * |
| deoxycorticosterone | | | | | | | | | |
| Corticosterone | 0.48 | 0.59 | 0.62 * | 0.38 * | 0.62 * | 0.33 * | 0.01 * | 0.02 * | 0 * |
| 21-Hydroxyprogesterone | 0.35 * | 0.45 * | 0.32 * | 0.5 * | 0.64 | 0.15 * | 0.05 * | 0.1 * | 0.04 |
| (11-Deoxycorticosterone) | | | | | | | | | |

TABLE 7
Markers for Hormone (estronenic) in female rats; Significant down-regulation changes
(p-Value ≦ 0.2) are marked (*). For some metabolites (marked with #), additional
information are provided in table 19. All compounds were administered in low dose.

| | 17-alpha- | | | | | |
|---|---|---|---|---|---|---|
| | Ethynylestradiol | | | Genistein | | |
| Metabolite | f7 | f14 | f28 | f7 | f14 | f28 |
| Behenic acid (C22:0) | 0.32 * | 0.52 * | 0.72 * | 0.45 * | 0.52 * | 0.57 * |
| Arachidonic acid | 0.23 * | 0.48 * | 0.63 * | 0.59 * | 0.66 * | 0.46 * |
| (C20:cis[5,8,11,14]4) | | | | | | |
| Nervonic acid (C24:cis[15]1) | 0.42 * | 0.44 * | 0.75 * | 0.45 * | 0.7 * | 0.43 * |
| erythro-Sphingosine (d18:1) | 0.16 * | 0.37 * | 0.65 * | 0.29 * | 0.61 * | 0.51 * |
| myo-Inositol, lipid fraction | 0.29 * | 0.61 * | 0.8 * | 0.62 * | 0.76 * | 0.75 * |
| Cholesterol | 0.22 * | 0.52 * | 0.65 * | 0.55 * | 0.66 * | 0.43 * |
| 5-O-Methylsphingosine (d18:1)# | 0.2 * | 0.35 * | 0.69 * | 0.27 * | 0.58 * | 0.47 * |
| Tricosanoic acid (C23:0) | 0.25 * | 0.44 * | 0.56 * | 0.32 * | 0.65 * | 0.42 * |
| Docosahexaenoic acid | 0.24 * | 0.45 * | 0.58 * | 0.52 * | 0.71 * | 0.43 * |
| (C22:cis[4,7,10,13,16,19]6) | | | | | | |
| Galactose, lipid fraction | 0.29 * | 0.56 * | 0.74 * | 0.51 * | 0.66 * | 0.62 * |
| Uric acid | 0.72 * | 0.67 * | 0.82 * | 0.73 * | 0.72 * | 0.57 * |
| threo-Sphingosine (d18:1) | 0.19 * | 0.44 * | 0.76 * | 0.33 * | 0.65 * | 0.57 * |
| 3-O-Methylsphingosine (d18:1)# | 0.17 * | 0.33 * | 0.64 * | 0.25 * | 0.55 * | 0.44 * |
| 16-Methylheptadecanoic acid | 0.53 * | 0.58 * | 0.73 * | 0.64 * | 0.72 * | 0.69 * |
| Sphingomyelin (d18:2, C16:0)# | 0.31 * | 0.58 * | 0.72 * | 0.55 * | 0.74 * | 0.62 * |
| Choline plasmalogen No 03# | 0.55 * | 0.57 * | 0.65 * | 0.74 * | 0.8 * | 0.57 * |
| myo-Inositol-2-phosphate, | 0.2 * | 0.6 * | 0.72 * | 0.48 * | 0.66 | 0.54 * |
| lipid fraction | | | | | | |
| Stearic acid (C18:0) | 0.39 * | 0.77 * | 0.95 * | 0.63 * | 0.83 * | 0.66 * |
| Lignoceric acid (C24:0) | 0.23 * | 0.44 * | 0.63 * | 0.45 * | 0.53 * | 0.44 * |
| 17-Methyloctadecanoic acid | 0.44 * | 0.66 * | 0.78 * | 0.7 * | 0.73 * | 0.86 * |
| Lysophosphatidylcholine (C17:0)# | 0.42 * | 0.73 * | 0.75 * | 0.8 * | 0.9 * | 0.81 * |
| alpha-Tocopherol | 0.16 * | 0.46 * | 0.58 * | 0.5 * | 0.65 * | 0.46 * |
| Malate | 0.6 * | 0.75 * | 0.64 * | 0.98 * | 0.75 * | 0.66 * |

TABLE 8a
Markers for Hormones (Estrogen Rez. Modulator) in female
rats; Significant up-regulation changes (p-Value ≦
0.1) are marked (*). For some metabolites (marked with
#), additional information are provided in table 19.
All compounds were administered in high dose.

| | | | | Raloxifen | | |
|---|---|---|---|---|---|---|
| | Tamoxifen | | | Hydrochloride | | |
| Metabolite | f7 | f14 | f28 | f7 | f14 | f28 |
| Coenzyme Q9 | 1.68 * | 1.49 * | 1.63 * | 1.18 * | 1.29 * | 1.31 * |
| TAG | 2.14 * | 1.9 * | 1.4 * | 1.84 * | 1.31 * | 1.64 * |
| (C18:2, | | | | | | |
| C18:3)# | | | | | | |
| Proline | 1.08 * | 1.05 * | 0.99 | 1.16 * | 1.2 * | 1.14 * |
| Glycine | 1.23 * | 1.26 * | 1.32 * | 1.53 * | 1.51 * | 1.4 * |
| Phosphatidyl- | 1.12 * | 1.19 * | 1.16 * | 1.17 * | 1.25 * | 1.26 * |
| choline | | | | | | |
| (C16:0, | | | | | | |
| C20:5)# | | | | | | |
| TAG | 2.67 * | 2.73 * | 2.07 * | 2.05 * | 1.27 * | 1.95 * |
| (C16:0, | | | | | | |
| C18:2)# | | | | | | |
| Thyroxine (T4) | 1.51 * | 0.7 * | 1.43 * | 1.36 | 2.5 * | 1.57 * |
| TAG No 059# | 1.91 * | 1.43 * | 1.89 * | 1.61 * | 1.47 * | 1.61 * |
| TAG (DAG- | 3 * | 2.73 * | 2.37 * | 1.8 * | 1.4 * | 1.76 * |
| Fragment)# | | | | | | |

TABLE 8b
Markers for Hormones (Estrogen Rez. Modulator) in female
rats; Significant down-regulation changes (p-Value ≦
0.1) are marked (*). For some metabolites (marked with
#), additional information are provided in table 19.
All compounds were administered in high dose.

| | | | | Raloxifene | | |
|---|---|---|---|---|---|---|
| | Tamoxifen | | | Hydrochloride | | |
| Metabolite | f7 | f14 | f28 | f7 | f14 | f28 |
| Phosphatidyl- | 0.81 * | 0.89 * | 0.88 * | 0.73 * | 0.86 * | 0.81 * |
| choline | | | | | | |
| (C18:0, | | | | | | |
| C20:3)# | | | | | | |
| Lysophospha- | 0.76 * | 0.87 * | 0.89 | 0.74 * | 0.72 * | 0.72 * |
| tidylcholine | | | | | | |
| (C17:0)# | | | | | | |
| Phosphatidyl- | 0.87 | 0.84 * | 0.76 * | 0.87 * | 0.85 * | 0.72 * |
| choline | | | | | | |
| (C18:0, | | | | | | |
| C22:6)# | | | | | | |
| Lysine | 0.89 | 0.6 * | 0.74 * | 0.8 * | 0.61 * | 0.81 * |

TABLE 9
Markers for Hormones (GnRH agonist) in male rats; Significant down-regulation changes
(p-Value ≦ 0.2) are marked (*). For some metabolites (marked with #), additional
information are provided in table 19. All compounds were administered in high dose.

| | Leuprolide | | | Medroxyprogesterone | | | | | |
|---|---|---|---|---|---|---|---|---|---|
| | acetate salt | | | acetat | | | Mibolerone | | |
| Metabolite | m7 | m14 | m28 | m7 | m14 | m28 | m7 | m14 | m28 |
| Testosterone | 0.35 * | 0.31 * | 0.3 * | 2 | 0.22 * | 0.08 * | 0.07 * | 0.68 * | 0.26 * |
| Androstenedione | 0.5 * | 0.56 * | 0.78 | 1.68 | 0.28 * | 0.1 * | 0.05 * | 0.96 | 0.46 * |
| Uric acid | 0.91 * | 0.62 * | 0.68 * | 0.6 * | 1.07 | NA | 0.66 * | 0.7 * | 0.5 * |
| Glycine | 0.87 * | 0.89 * | 0.99 | 1.05 * | 1.06 | NA | 0.86 * | 0.86 | 0.72 * |
| Heptadecanoic | 0.91 * | 1 | 0.84 * | 0.98 | 0.92 | NA | 0.37 * | 0.57 * | 0.82 * |
| acid (C17:0) | | | | | | | | | |
| alpha- | 0.9 * | 0.97 * | 0.96 | 0.82 * | 0.82 * | NA | 0.07 * | 0.33 * | 0.64 * |
| Tocopherol | | | | | | | | | |
| Alanine | 0.72 * | 0.72 * | 0.81 * | 0.73 * | 0.97 | NA | 0.72 * | 0.93 * | 1.14 |
| Threonine | 0.78 * | 0.77 * | 0.87 * | 0.82 * | 1 | NA | 0.76 * | 1.02 | 1.04 |
| Cholesterol | 0.81 * | 0.89 | 0.9 | 0.9 | 0.69 * | NA | NA | 0.27 * | 0.52 * |

| | Napthyl- | Norethin- | | | | | | |
|---|---|---|---|---|---|---|---|---|
| | isothio- | drone | Buserelin | | | | | |
| | cyanate | acetate | acetate a.c. | | | Ifosfamide | | |
| Metabolite | m7 | m7 | m7 | m14 | m28 | m7 | m14 | m28 |
| Testosterone | 0.26 * | 0.59 * | 0.11 * | 0.19 * | 0.08 * | 2.35 | 0.21 * | 0.22 * |
| Androstenedione | 0.5 * | 0.42 * | 0.17 * | 0.5 * | 0.35 * | 3.68 | 0.21 * | 0.24 * |
| Uric acid | 0.59 | 0.7 * | 0.59 * | 076 * | 0.85 * | 0.57 | NA | 0.65 |
| Glycine | 0.76 | 0.86 * | 0.83 * | 0.91 | 0.91 * | 1.33 | NA | 1.28 |
| Heptadecanoic | 2.56 | 0.56 * | 0.81 * | 0.94 * | 0.91 * | 0.94 | NA | 1.08 |
| acid (C17:0) | | | | | | | | |
| alpha- | 2.64 | 0.32 * | 0.88 * | 0.93 * | 1.03 | 0.99 | NA | 0.95 |
| Tocopherol | | | | | | | | |
| Alanine | 0.72 | 0.88 * | 0.67 * | 0.74 * | 0.78 | 1.07 | NA | 0.76 |
| Threonine | 1.08 | 0.88 * | 1.01 * | 0.92 * | 1.01 | 1.21 | NA | 0.88 |
| Cholesterol | 4.86 | 0.28 * | 0.9 * | 0.84 * | 0.91 | 1.12 | NA | 1.13 |

TABLE 10a
Markers for Hormones (antiandrogen) in male rats; Significant up-regulation changes
(p-Value ≦ 0.1) are marked (*). For some metabolites (marked with #), additional
information are provided in table 19. All compounds were administered in high dose.

| | Iprodione | | | Metazachlor | | | Methimazole | | |
|---|---|---|---|---|---|---|---|---|---|
| Metabolite | m7 | m14 | m28 | m7 | m14 | m28 | m7 | m14 | m28 |
| threo- | 1.18 * | 1.24 | 1.41 * | 2.18 * | 3.13 * | 3.33 * | 2.91 * | 3.72 * | 4.28 * |
| Sphingosine | | | | | | | | | |
| (d18:1) | | | | | | | | | |
| 3-O- | 1.23 * | 1.28 * | 1.36 * | 2.88 * | 4.31 * | 4.4 * | 3.59 * | 3.98 * | 4.59 * |
| Methylsphingosine | | | | | | | | | |
| (d18:1)# | | | | | | | | | |
| Lignoceric acid | 1.11 | 1.29 | 1.38 * | 1.6 * | 2.38 * | 2.11 * | 2.12 * | 2.82 * | 2.16 * |
| (C24:0) | | | | | | | | | |
| erythro- | 1.25 * | 1.13 * | 1.43 * | 2.3 * | 3.47 * | 3.68 * | 2.91 * | 3.71 * | 3.44 * |
| Sphingoside | | | | | | | | | |
| (d18:1) | | | | | | | | | |
| 5-O- | 1.25 * | 1.15 * | 1.4 * | 3.04 * | 4.66 * | 4.51 * | 3.41 * | 4.75 * | 4.37 * |
| Methylsphingosine | | | | | | | | | |
| (d18:1)# | | | | | | | | | |
| Stearic acid | 1.13 | 1.04 | 1.33 * | 1.45 * | 2.36 * | 2.59 * | 1.34 * | 1.4 * | 1.16 * |
| (C18:0) | | | | | | | | | |
| Nervonic acid | 1.49 * | 1.28 * | 1.37 * | 3.17 * | 4 * | 3.63 * | 2.59 * | 3 * | 2.23 * |
| (C24:cis[15]1) | | | | | | | | | |
| Cysteine | 1.36 * | 1.02 | 1.19 | 1.07 | 0.86 | 1.41 * | 0.74 | 1.25 | 0.92 |
| Oleic acid | 0.98 | 0.89 | 1.24 * | 1.09 | 1.82 * | 1.82 * | 0.95 | 0.97 | 0.87 |
| (C18:cis[9]1) | | | | | | | | | |
| alpha-Tocopherol | 1.18 | 1 | 1.27 * | 1.71 * | 2.2 * | 2.11 * | 1.76 * | 2.66 * | 2.18 * |

| | | | | | | | Fluoxetine | | |
|---|---|---|---|---|---|---|---|---|---|
| | Vinclozolin | | | Flutamide | | | hydrochloride | | |
| Metabolite | m7 | m14 | m28 | m7 | m14 | m28 | m7 | m14 | m28 |
| threo- | 1.63 * | 1.53 * | 1.34 * | 1.39 * | 0.95 | 1.38 * | 1.2 | 1.45 * | 2.2 * |
| Sphingosine | | | | | | | | | |
| (d18:1) | | | | | | | | | |
| 3-O- | 1.63 * | 1.39 * | 1.9 * | 1.27 * | 0.78 | 1.52 | 1.33 | 1.43 | 2.23 * |
| Methylsphingosine | | | | | | | | | |
| (d18:1)# | | | | | | | | | |
| Lignoceric acid | 1.45 * | 1.25 * | 1.26 * | 1.43 * | 0.9 | 1.3 * | 1.33 | 1.35 | 1.79 * |
| (C24:0) | | | | | | | | | |
| erythro- | 1.51 * | 1.37 * | 1.61 * | 1.28 * | 0.79 | 1.31 | 1.07 | 1.56 * | 2.12 * |
| Sphingoside | | | | | | | | | |
| (d18:1) | | | | | | | | | |
| 5-O- | 1.65 * | 1.51 * | 1.72 * | 1.24 * | 0.81 | 1.43 | 1.43 | 1.67 * | 2.39 * |
| Methylsphingosine | | | | | | | | | |
| (d18:1)# | | | | | | | | | |
| Stearic acid | 1.07 | 1.3 * | 1.16 * | 1.15 * | 1.01 | 1.29 * | 1.21 | 1.2 | 1.42 * |
| (C18:0) | | | | | | | | | |
| Nervonic acid | 1.74 * | 1.93 * | 1.63 * | 1.39 * | 0.93 | 1.46 * | 1.37 | 1.68 | 1.57 * |
| (C24:cis[15]1) | | | | | | | | | |
| Cysteine | 2.38 * | 2.49 * | 2.41 * | 1.21 | 0.96 | 1.41 * | 1.81 * | 2.03 * | 1.37 * |
| Oleic acid | 0.86 | 0.81 | 0.88 | 1.37 * | 1.1 | 1.14 | 0.83 | 0.88 | 0.98 |
| (C18:cis[9]1) | | | | | | | | | |
| alpha-Tocopherol | 1.21 | 1.44 * | 1.51 * | 1.55 * | 1.24 | 1.54 * | 1.37 | 1.64 * | 1.74 * |

TABLE 10b
Markers for Hormones (antiandrogen) in male rats; Significant down-regulation changes
(p-Value ≦ 0.1) are marked (*). For some metabolites (marked with #), additional
information are provided in table 19. All compounds were administered in high dose.

| | Iprodione | | | Metazachlor | | | Methimazole | | | Vinclozolin |
|---|---|---|---|---|---|---|---|---|---|---|
| Metabolite | m7 | m14 | m28 | m7 | m14 | m28 | m7 | m14 | m28 | m7 |
| trans-4- | 0.84 * | 0.92 | 0.96 | 0.93 | 0.72 * | 0.84 * | 0.85 * | 0.76 * | 0.92 | 0.81 * |
| Hydroxyproline | | | | | | | | | | |
| Thyroxine (T4) | 0.15 * | 0.53 * | 0.4 * | 0.98 | 0.56 * | 0.74 | 0.11 * | 0.04 * | 0.02 * | 0.62 |
| Phosphatidylcholine | 0.85 | 0.94 | 0.96 * | 0.98 | 0.83 * | 0.79 * | 1.13 * | 1.09 | 0.92 * | 1.01 |
| (C16:0, C16:0)# | | | | | | | | | | |
| Oleic acid | 0.98 | 0.89 | 1.24 | 1.09 | 1.82 | 1.82 | 0.95 | 0.97 | 0.87 * | 0.86 * |
| (C18:cis[9]1) | | | | | | | | | | |

| | | | | | | Fluoxetine | | |
|---|---|---|---|---|---|---|---|---|
| | Vinclozolin | | Flutamide | | | hydrochloride | | |
| Metabolite | m14 | m28 | m7 | m14 | m28 | m7 | m14 | m28 |
| trans-4- | 0.79 * | 0.85 * | 0.79 * | 0.89 * | 0.72 * | 0.87 * | 0.83 * | 0.72 * |
| Hydroxyproline | | | | | | | | |
| Thyroxine (T4) | 0.38 | 0.4 * | 0.46 * | 0.57 | 0.72 | 0.42 * | 0.59 | 0.72 |
| Phosphatidylcholine | 0.93 | 0.9 * | 1.12 | 0.7 * | 0.81 * | 0.99 | 1.03 | 0.99 |
| (C16:0, C16:0)# | | | | | | | | |
| Oleic acid | 0.81 | 0.88 * | 1.37 | 1.1 | 1.14 | 0.83 | 0.88 * | 0.98 |
| (C18:cis[9]1) | | | | | | | | |

TABLE 11a
Markers for hormones (antiandrogen receptor antagonist prostate) in male rats; Significant up-
regulation changes (p-Value ≦ 0.2) are marked (*). For some metabolites (marked with #), additional
information are provided in table 19. All compounds were administered in high dose.

| | Cyproteron | | | | | | | | |
|---|---|---|---|---|---|---|---|---|---|
| | Acetate | | | Flutamide | | | Vinclozolin | | |
| Metabolite | m7 | m14 | m28 | m7 | m14 | m28 | m7 | m14 | m28 |
| Tryptophan | 1.1 * | 1.11 * | 1.14 * | 1.22 * | 1.15 | 1.08 * | 1.13 * | 1.12 * | 1.04 |
| Threonine | 1.78 * | 2.58 * | 1.73 * | 1.33 * | 1.24 * | 1.22 * | 1.16 * | 1.08 | 1.16 * |
| Lignoceric acid | 1.46 * | 1.78 * | 1.9 * | 1.43 * | 0.9 | 1.3 * | 1.45 * | 1.25 * | 1.26 * |
| (C24:0) | | | | | | | | | |
| Nervonic acid | 1.69 * | 2.55 * | 2.83 * | 1.39 * | 0.93 | 1.46 * | 1.74 * | 1.93 * | 1.63 * |
| (C24:cis[15]1) | | | | | | | | | |
| Behenic acid | 1.38 * | 1.52 * | 1.57 * | 1.89 * | 1.12 * | 1.34 * | 1.26 * | 1.31 * | 1.29 |
| (C22:0) | | | | | | | | | |
| Stearic acid (C18:0) | 1.44 * | 2.3 * | 2.71 * | 1.15 * | 1.01 | 1.29 * | 1.07 * | 1.3 * | 1.16 * |
| Dodecanol | 1.26 * | 1.71 * | 1.52 * | 1.2 * | 1.09 * | 1.25 * | 1.1 | 1.16 * | 0.95 |
| Galactose, lipid fraction | 1.37 * | 2.57 * | 1.68 * | 1.44 * | 1.07 | 1.23 * | 1.21 * | 1.23 * | 1.19 * |
| Phosphate, lipid | 1.32 * | 1.8 * | 1.86 * | 1.35 * | 1.08 | 1.17 * | 1.11 | 1.21 * | 1.05 |
| fraction | | | | | | | | | |
| Androstenedione | 4.65 | 3.8 * | 0.94 | 19.42 * | 46.48 * | 18.79 * | 2.11 * | 3.46 * | 1.78 * |
| alpha-Tocopherol | 1.22 * | 1.32 * | 1.44 * | 1.43 * | 1.16 | 1.19 | 1.28 * | 1.14 * | 1.33 * |
| Indole-3-lactic acid | 1.13 * | 1.15 * | 1.15 * | 1.25 * | 1.06 | 1.16 | 1.23 * | 1.06 | 1.09 * |
| myo-Inositol-2- | 1.65 * | 2.78 * | 3.26 * | 2.52 * | 1.06 * | 1.44 * | 1.39 * | 1.43 | 1.49 * |
| phosphate, lipid fraction | | | | | | | | | |
| Tricosanoic acid | 1.48 * | 1.3 * | 1.39 * | 1.16 * | 0.9 | 1.05 | 1.29 * | 1.14 * | 1.21 |
| (C23:0) | | | | | | | | | |
| Phytosphingosine | 1.05 | 1.23 * | 1.68 * | 2.29 * | 1.28 * | 1.26 * | 1.28 * | 1.33 * | 1.22 * |
| Arachidonic acid | 1.35 * | 2.03 * | 2.35 * | 1.28 * | 0.99 | 1.21 * | 1.02 | 1.07 * | 1.08 |
| (C20:cis[5,8,11,14]4) | | | | | | | | | |
| Glycerol phosphate, | 1.57 * | 2.05 * | 2.16 * | 1.57 * | 1.18 * | 1.28 * | 1.08 * | 1.04 | 1.06 |
| lipid fraction# | | | | | | | | | |
| Testosterone | 3.32 | 2.63 * | 0.56 | 10.33 * | 16.89 * | 8.45 * | 1.41 | 2.44 * | 1.09 |
| 3-Methoxytyrosine | 1.37 * | 1.81 * | 2.05 * | 1.97 * | 1.47 * | 1.55 * | 1.33 * | 1.16 * | 1.11 * |
| Cholesterol | 1.33 * | 1.63 * | 1.97 * | 1.43 * | 1.07 | 1.22 * | 1.02 | 1.1 * | 1.03 |
| 3-O-Methylsphingosine | 1.73 * | 2.19 * | 2.98 * | 1.27 * | 0.78 | 1.52 * | 1.63 * | 1.39 * | 1.9 * |
| (d18:1)# | | | | | | | | | |
| threo-Sphingosine | 1.39 * | 2 * | 2.13 * | 1.39 * | 0.95 | 1.38 * | 1.63 * | 1.53 * | 1.34 * |
| (d18:1) | | | | | | | | | |
| erythro-Sphingosine | 1.68 * | 1.92 * | 2.2 * | 1.28 * | 0.79 | 1.31 * | 1.51 * | 1.37 * | 1.61 * |
| (d18:1) | | | | | | | | | |
| 5-O-Methylsphingosine | 1.83 * | 2.18 * | 2.54 * | 1.24 * | 0.81 | 1.43 * | 1.65 * | 1.51 * | 1.72 * |
| (d18:1)# | | | | | | | | | |
| Cysteine | 1.3 * | 1.28 * | 1.54 * | 1.21 | 0.96 | 1.41 * | 2.38 * | 2.9 * | 2.41 * |
| Phosphatidylcholine | 1.03 | 1.16 * | 1.18 * | 1.14 * | 1.01 | 0.94 | 1.01 | 1.11 * | 0.98 |
| (C18:0, C20:3)# | | | | | | | | | |
| Phosphatidylcholine | 1.09 * | 1.48 * | 1.55* | 1.43 * | 1.15 | 1.05 | 1.29 * | 1.3 | 1.13 * |
| (C18:0, C18:1)# | | | | | | | | | |
| Noradrenaline | 1.61 * | 1.22 | 1.39 * | 1.36 * | 1.85 * | 0.93 | 0.96 | 0.93 | 1.18 |
| (Norepinephrine) | | | | | | | | | |
| Phosphatidylcholine | 1.31 * | 1.5 * | 1.67 * | 1.01 | 0.96 | 1.34 * | 1.1 * | 1.46 * | 1.17 * |
| (C18:0, C22:6)# | | | | | | | | | |
| Phosphatidylcholine | 1.04 * | 1.04 | 1.03 | 1.38 * | 1.17 | 1.13 | 1.13 * | 1.08 * | 1.05 |
| (C18:0, C18:2)# | | | | | | | | | |
| beta-Sitosterol | 2.11 * | 2.16 * | 2.6 * | 1.24 * | 0.91 | 0.89 | 0.88 | 1.11 * | 0.82 |
| dihomo-gamma- | 1.66 * | 2.78 * | 4.01 * | 1.55 * | 1.46 * | 2.58 * | 2.04 * | 1.59 * | 1.77 * |
| Linolenic acid | | | | | | | | | |
| (C20:cis[8,11,14]3) | | | | | | | | | |
| Eicosanoic acid | 1.06 | 1.15 | 1.71 * | 2.11 * | 1.84 * | 1.93 * | 1.25 | 1.26 * | 1.05 |
| (C20:0) | | | | | | | | | |
| Histidine | 1.39 * | 1.17 | 1.37 * | 1.04 | 1.12 * | 0.98 | 1.56 * | 1.16 * | 1.04 |

TABLE 11b
Markers for hormones (antiandrogen receptor antagonist prostate) in male rats; Significant down-
regulation changes (p-Value ≦ 0.2) are marked (*). For some metabolites (marked with #), additional
information are provided in table 19. All compounds were administered in high dose.

| | Cyproteron | | | | | | | | |
|---|---|---|---|---|---|---|---|---|---|
| | Acetate | | | Flutamide | | | Vinclozolin | | |
| Metabolite | m7 | m14 | m28 | m7 | m14 | m28 | m7 | m14 | m28 |
| 3-Hydroxybutyrate | 0.58 * | 0.73 * | 0.68 * | 0.53 * | 0.72 * | 1.08 | 0.71 * | 0.59 | 0.85 |
| Progesterone | 0.4 * | 0.54 * | 0.03 * | 0.64 | 0.33 * | 0.25 * | 0.18 * | 0.18 * | 0.43 * |
| Phosphatidylcholine | 0.78 * | 0.87 * | 0.79 * | 1.12 | 0.7 * | 0.81 * | 1.01 | 0.93 | 0.9 * |
| (C16:0, C16:0)# | | | | | | | | | |
| trans-4-Hydroxyproline | 0.53 * | 0.44 * | 0.52 * | 0.79 * | 0.89 * | 0.72 * | 0.95 | 0.79 * | 0.85 * |
| 21-Hydroxyprogesterone | 0.3 * | 0.41 * | 0.01 * | 0.65 | 0.31 * | 0.26 * | 0.18 * | 0.09 * | 0.21 * |
| (11-Deoxycorticosterone) | | | | | | | | | |
| Coenzyme Q10 | 0.49 * | 0.86 | 0.7 * | 0.58 * | 0.43 * | 0.4 * | 0.56 * | 0.46 * | 0.67 |
| Coenzyme Q9 | 0.58 * | 0.74 * | 0.59 * | 0.73 * | 1.04 | 0.95 | 0.82 | 0.86 * | 0.79 |
| Palmitoleic acid | 0.88 * | 0.74 * | 1.42 * | 0.77 * | 0.68 * | 0.6 * | 0.56 * | 0.7 * | 0.73 * |
| (C16:cis[9]1) | | | | | | | | | |
| 18-Hydroxy-11- | 0.03 * | 0.01 * | 0 * | 0.75 | 0.48 * | 0.07 * | 0.07 * | 0.12 * | 0.23 * |
| deoxycorticosterone | | | | | | | | | |
| Corticosterone | 0.04 * | 0.01 * | 0 * | 0.83 | 0.38 * | 0.07 * | 0.25 * | 0.14 * | 0.58 * |
| Metanephrine | 0.91 | 0.66 * | 0.58 * | 0.5 * | 0.61 * | 0.48 * | 1 | 0.87 | 0.84 |
| 4-Hydroxy-3- | 1.11 | 0.99 | 0.82 * | 0.67 * | 0.7 * | 0.8 * | 1.09 * | 1.01 | 0.95 * |
| methoxyphenylglycol | | | | | | | | | |
| (HMPG) | | | | | | | | | |
| Citrate | 1.24 | 0.73 * | 0.81 | 0.74 * | 0.88 * | 0.9 | 0.81 * | 0.85 * | 0.74 * |

TABLE 12a
Markers for Hormones (Estrogens) in female rats; Significant
up-regulation changes (p-Value ≦ 0.2) are marked (*). For some
metabolites (marked with #), additional information are provided in
table 19. All compounds were administered in high dose.

| | 17-alpha- | | |
|---|---|---|---|
| | Ethynylestradiol | | |
| Metabolite | f7 | f14 | f28 |
| Proline | 1.04 * | 1.04 * | 1.15 * |
| Threonine | 1.19 * | 1.21 * | 1.42 * |
| Mannose | 1.47 * | 1.37 * | 1.48 * |
| Histidine | 1.18 * | 1.42 * | 1.31 * |
| Serine | 1.15 * | 1.22 * | 1.2 * |
| Alanine | 0.93 | 1.09 * | 1.12 |
| TAG | 2.35 * | 5.85 * | 3.25 * |
| (C16:0, C18:1, C18:3)# | | | |
| TAG (C18:2, C18:2)# | 1.85 | 3.16 * | 1.98 * |
| TAG | 1.52 * | 2.65 * | 1.52 * |
| (C18:3, C18:2, C18:1)# | | | |
| Glucose | 1.37 * | 1.13 | 1.31 * |

TABLE 12b
Markers for Hormones (Estrogens) in female rats; Significant
down-regulation changes (p-Value ≦ 0.2) are marked (*). For
some metabolites (marked with #), additional information are
provided in table 19. All compounds were administered in high dose.

| | 17-alpha- | | |
|---|---|---|---|
| | Ethynylestradiol | | |
| Metabolite | f7 | f14 | f28 |
| Choline plasmalogen (C18, C20:4) | 0.19 * | 0.32 * | 0.38 * |
| Phosphatidylcholine (C18:0, C20:4)# | 0.63 * | 0.86 * | 0.95 * |
| Sphingomyelin (d18:2, C18:0)# | 0.12 * | 0.27 * | 0.24 * |
| Choline plasmalogen No 02# | 0.21 * | 0.41 * | 0.42 * |
| Lysophosphatidylcholine (C18:0)# | 0.83 * | 0.89 * | 0.91 * |
| Phosphatidylcholine (C18:0, C22:6)# | 0.28 * | 0.42 * | 0.5 * |
| Tyrosine | 0.73 * | 0.69 * | 0.74 * |
| Arachidonic acid | NA | 0.3 * | 0.27 * |
| (C20:cis[5,8,11,14]4) | | | |
| Lysophosphatidylcholine (C17:0)# | 0.39 * | 0.51 * | 0.55 * |
| alpha-Tocopherol | 0.27 * | 0.33 * | 0.41 * |
| Sphingomyelin (d18:1, C16:0) | 0.27 * | 0.52 * | 0.54 * |
| Isopalmitic acid (C16:0) | NA | 0.55 * | 0.51 * |
| Choline plasmalogen No 03# | 0.25 * | 0.48 * | 0.38 * |
| Sphingomyelin (d18:1, C23:0)# | 0.15 * | 0.34 * | 0.37 * |
| Docosahexaenoic acid | NA | 0.21 * | 0.24 * |
| (C22:cis[4,7,10,13,16,19]6) | | | |
| Lysophosphatidylethanolamine | 0.34 * | 0.42 * | 0.48 * |
| (C22:0) | | | |
| TAG No 02# | 0.24 * | 0.17 * | 0.31 * |
| Behenic acid (C22:0) | NA | 0.31 * | 0.37 * |
| Lignoceric acid (C24:0) | NA | 0.28 * | 0.25 * |
| 3-O-Methylsphingosine (d18:1)# | NA | 0.15 * | 0.17 * |
| Metanephrine | 0.84 | 0.67 * | 0.53 * |
| erythro-Sphingosine (d18:1) | NA | 0.12 * | 0.16 * |
| Phosphatidylcholine No 04# | 0.19 * | 0.62 * | 0.39 * |
| Sphingomyelin (d18:2, C16:0)# | 0.18 * | 0.3 * | 0.22 * |
| 5-O-Methylsphingosine (d18:1)# | NA | 0.16 * | 0.14 * |
| Hippuric acid | 0.95 | 0.68 * | 0.31 * |
| Lactate | 0.57 * | 0.71 * | 0.99 |
| Coenzyme 010 | 0.49 * | 0.91 | 0.81 * |
| 16-Methylheptadecanoic acid | NA | 0.31 * | 0.29 * |
| 17-Methyloctadecanoic acid | NA | 0.43 * | 0.46 * |
| Stearic acid (C18:0) | NA | 0.52 * | 0.44 * |
| Campesterol | NA | 0.45 * | 0.54 |

TABLE 12c
Markers for Hormones (estrogen) in male rats;
Significant up-regulation changes (p-Value ≦ 0.2) are marked (*).
For some metabolites (marked with #), additional information are
providedin table 19. All compounds were administered in high dose.

| | 17-alpha- | | |
|---|---|---|---|
| | Ethynylesbadiol | | |
| Metabolite | m7 | m14 | m28 |
| Mannose | 1.34 * | 1.25 * | 1.2 * |
| 1,5-Anhydrosorbitol | 1.22 * | 1.56 * | 1.54 * |
| Lysine | 1.2 | 1.29 * | 1.17 * |
| Serine | 1.21 * | 1.33 * | 1.29 * |
| Proline | 1.06 | 1.29 * | 1.19 * |
| Alanine | 1.09 | 1.48 * | 1.29 * |
| Threonine | 1.22 * | 1.46 * | 1.22 * |
| Serotonin (5-HT) | 3.71 * | 3.13 * | 2.48 * |
| Histidine | 1.34 * | 1.52 * | 1.23 * |

TABLE 12d
Markers for Hormones (estrogen) in male rats; Significant
down-regulation changes (p-Value ≦ 0.2) are marked (*).
For some metabolites (marked with #), additional information
are provided in table 19. All compounds were administered in high dose.

| | 17-alpha- | | |
|---|---|---|---|
| | Ethynylestradiol | | |
| Metabolite | m7 | m14 | m28 |
| Sphingomyelin (d18:2, C16:0)# | 0.19 * | 0.24 * | 0.34 * |
| Choline plasmalogen No 02# | 0.28 * | 0.46 * | 0.55 * |
| Lysophosphatidylcholine (C18:0)# | 0.77 * | 0.84 * | 0.89 * |
| Lysophosphatidylethanolamine | 0.28 * | 0.45 * | 0.54 * |
| (C22:0) | | | |
| Threonic acid | 0.47 * | 0.52 * | 0.54 * |
| Sphingomyelin (d18:2, C18:0)# | 0.24 * | 0.18 * | 0.41 * |
| Lysophosphatidylcholine (C20:4)# | 0.78 * | 0.83 * | 0.88 * |
| Lysophosphatidylcholine (C17:0)# | 0.38 * | 0.58 * | 0.67 * |
| Sphingomyelin (d18:1, C16:0)# | 0.32 * | 0.4 * | 0.65 * |
| Coenzyme Q10 | 0.16 * | 0.31 * | 0.37 * |
| Phosphatidylcholine | 0.21 * | 0.47 * | 0.79 * |
| (C18:0, C20:3)# | | | |
| beta-Sitosterol | 0.3 * | 0.38 * | 0.7 * |
| Phosphatidylcholine No 04# | 0.17 * | 0.42 * | 0.64 * |
| myo-Inositol, lipid fraction | 0.22 * | 0.29 * | 0.53 * |
| Glycerol phosphate, lipid fraction# | 0.22 * | 0.31 * | 0.29 * |
| Glycerol, polar fraction | 0.68 * | 0.84 | 0.63 * |
| 3,4-Dihydroxyphenylacetic acid | 1.01 | 0.58 * | 0.96 |
| (DOPAC) | | | |
| Choline plasmalogen No 03# | 0.41 * | 0.43 * | 0.48 * |
| Elaidic acid (C18:trans[9]1) | 0.54 * | 0.63 * | 0.48 * |
| Tyrosine | 1.09 | 0.96 | 0.83 * |

TABLE 13a
Markers for Hormones (putative anti-prolactin) in female rats; Significant
up-regulation changes (p-Value ≦ 0.2) are marked (*). For some
metabolites (marked with #), additional information are provided
in table 19. All compounds were administered in high dose.

| | Bromocriptine | | | | | |
|---|---|---|---|---|---|---|
| | Mesylate | | | Cabergoline | | |
| Metabolite | f7 | f14 | f28 | f7 | f14 | f28 |
| 3-Hydroxybutyrate | 1.19 | 1.11 | 1.13 | 1.31 * | 1.32 * | 1.01 |

TABLE 13b
Markers for Hormones (putative anti-prolactin) in female rats; Significant
down-regulation changes (p-Value ≦ 0.2) are marked (*). For
some metabolites (marked with #), additional information are
provided in table 19. All compounds were administered in high dose.

| | Bromocriptine | | | | | |
|---|---|---|---|---|---|---|
| | Mesylate | | | Cabergoline | | |
| Metabolite | f7 | f14 | f28 | f7 | f14 | f28 |
| Ceramide (d18:1, C24:1) | 0.7 * | 0.59 * | 0.6 * | 0.57 * | 0.6 * | 0.39 * |
| Cholesterolester No 01# | 0.83 * | 0.82 * | 0.85 * | 0.84 | 0.79 * | 0.56 * |
| myo-Inositol-2-phosphate, | 0.74 * | 0.42 * | 0.35 * | 0.61 * | 0.31 * | 0.08 * |
| lipid fraction | | | | | | |
| Phosphatidylcholine | 0.85 * | 0.7 * | 0.71 * | 0.85 * | 0.72 * | 0.59 * |
| (C18:0, C20:3)# | | | | | | |
| Ceramide (d18:1, C24:0) | 0.74 * | 0.72 * | 0.52 * | 0.53 * | 0.57 * | 0.36 * |
| myo-Inositol, lipid fraction | 0.77 * | 0.72 * | 0.54 * | 0.87 * | 0.48 * | 0.23 * |
| Stearic acid (C18:0) | 0.78 * | 0.66 * | 0.57 * | 0.79 * | 0.42 * | 0.28 * |
| TAG No 07# | 0.45 * | 0.71 * | 0.9 | 0.91 | 0.73 * | 0.4 * |
| Glycerol phosphate, lipid | 0.83 * | 0.6 * | 0.56 * | 0.72 * | 0.34 * | 0.19 * |
| fraction# | | | | | | |
| Sphingomyelin | 0.85 * | 0.87 * | 0.79 * | 0.94 | 0.79 * | 0.51 * |
| (d18:1, C16:0)# | | | | | | |
| Tricosanoic acid (C23:0) | 0.94 | 0.75 * | 0.51 * | 0.71 * | 0.55 * | 0.23 * |
| Heptadecanoic acid (C17:0) | 0.89 * | 0.7 * | 0.59 * | 0.85 | 0.61 * | 0.47 * |
| beta-Sitosterol | 0.81 * | 0.62 * | 0.24 * | 0.46 * | 0.3 * | 0.13 * |
| Arachidonic acid | 0.77 * | 0.64 * | 0.49 * | 0.71 * | 0.32 * | 0.16 * |
| (C20:cis[5,8,11,14]4) | | | | | | |
| Phytosphingosine | 0.79 * | 0.63 * | 0.46 * | 0.64 * | 0.55 * | 0.34 * |
| Lysophosphatidylcholine | 0.9 | 0.84 * | 0.81 * | 0.83 * | 0.72 * | 0.57 * |
| (C17:0)# | | | | | | |
| Sphingomyelin | 0.87 * | 0.71 * | 0.62 * | 0.89 * | 0.67 * | 0.3 * |
| (d18:2, C18:0)# | | | | | | |
| 3-O-Methylsphingosine | 0.83 * | 0.67 * | 0.34 * | 0.68 * | 0.36 * | 0.11 * |
| (d18:1)# | | | | | | |
| Sphingomyelin | 0.93 | 0.82 * | 0.53 * | 0.67 * | 0.58 * | 0.33 * |
| (d18:2, C16:0)# | | | | | | |
| Lignoceric acid (C24:0) | 0.81 * | 0.69 * | 0.43 * | 0.54 * | 0.33 * | 0.15 * |
| 16-Methylheptadecanoic | 0.81 * | 0.51 * | 0.51 * | 0.57 * | 0.59 * | 0.28 * |
| acid | | | | | | |
| Cholesterol | 0.77 * | 0.69 * | 0.48 * | 0.72 * | 0.36 * | 0.16 * |
| erythro-Sphingosine (d18:1) | 0.86 | 0.72 * | 0.36 * | 0.66 * | 0.37 * | 0.13 * |
| threo-Sphingosine (d18:1) | 0.77 * | 0.74 * | 0.4 * | 0.75 * | 0.44 * | 0.24 * |
| Galactose, lipid fraction | 0.78 * | 0.68 * | 0.57 * | 0.79 | 0.48 * | 0.24 * |
| 5-O-Methylsphingosine | 0.8 | 0.68 * | 0.36 * | 0.66 * | 0.34 * | 0.13 * |
| (d18:1)# | | | | | | |
| Phosphate, lipid fraction | 0.97 | 0.83 * | 0.71 * | 0.73 * | 0.57 * | 0.36 * |
| Docosahexaenoic acid | 0.94 | 0.76 * | 0.56 * | 1.07 | 0.48 * | 0.21 * |
| (C22:cis[4,7,10,13,16,19]6) | | | | | | |
| Elaidic acid | 1.05 | 0.73 * | 0.81 * | 0.66 * | 0.5 * | 0.33 * |
| (C18:trans[9]1) | | | | | | |
| Dodecanol | 0.89 | 0.66 * | 0.53 * | 0.84 * | 0.44 * | 0.27 * |
| Sphingomyelin | 0.82 * | 0.8 * | 0.81 * | 0.84 * | 0.62 * | 0.36 * |
| (d18:1, C24:0)# | | | | | | |
| alpha-Tocopherol | 0.8 * | 0.69 * | 0.49 * | 0.82 | 0.4 * | 0.16 * |

TABLE 14a
Markers for Hormones (Sexhormone) in female rats; Significant down-regulation changes
(p-Value ≦ 0.1) are marked (*). For some metabolites (marked with #), additional
information are provided in table 19. All compounds were administered in high dose.

| | Norethindrone | 17-alpha- | | | Diethylstilboestrol | | |
|---|---|---|---|---|---|---|---|
| | acetate | Ethynylestradiol | | | dipropionate | | |
| Metabolite | f7 | f7 | f14 | f28 | f7 | f14 | f28 |
| Choline plasmalogen | 0.3 * | 0.21 * | 0.41 * | 0.42 * | 0.7 * | 0.75 * | 0.67 * |
| No 02# | | | | | | | |
| Cholesterol | 0.17 * | NA | 0.29 * | 0.32 * | 0.48 * | 0.69 * | 0.58 * |
| erythro- | 0.03 * | NA | 0.12 * | 0.16 * | 0.38 * | 0.67 * | 0.46 * |
| Sphingosine | | | | | | | |
| (d18:1) | | | | | | | |
| Lignoceric acid | 0.27 | NA | 0.28 * | 0.25 * | 0.49 * | 0.6 * | 0.59 * |
| (C24:0) | | | | | | | |
| Sphingomyelin | 0.19 * | 0.18 * | 0.3 * | 0.22 * | 0.67 * | 0.71 * | 0.58 * |
| (d18:2, C16:0)# | | | | | | | |
| Sphingomyelin | 0.12 * | 0.21 * | 0.35 * | 0.29 * | 0.72 * | 0.59 | 0.73 * |
| (d18:1, C16:0) | | | | | | | |

TABLE 14b
Markers for Hormones (Sexhormone) in male rats; Significant down-regulation
changes (p-Value ≦ 0.1) are marked (*). For some metabolites (marked with #),
additional information are provided in table 19. All compounds were administered in high dose.

| | 17-alpha- | | | | | | 17-alpha- | | | | | | Raloxifene Hy- | | | | | |
|---|---|---|---|---|---|---|---|---|---|---|---|---|---|---|---|---|---|---|
| Meta- | Ethynylestradiol | | | Diethanolamine | | | Methyltestosterone | | | Tamoxifen | | | drochloride | | | Trenbolone | | |
| bolite | m7 | m14 | m28 | m7 | m14 | m28 | m7 | m14 | m28 | m7 | m14 | m28 | m7 | m14 | m28 | m7 | m14 | m28 |
| Choline | 0.28 | 0.46 | 0.55 | 0.89 | 0.68 | 0.74 | 0.55 | 0.64 | 0.49 | 0.59 | 0.6 | 0.68 | 0.67 | 0.69 | 0.65 | 0.44 | 0.52 | 0.55 |
| plasma- | * | * | * | | * | * | * | * | * | * | * | * | * | * | * | * | * | * |
| logen | | | | | | | | | | | | | | | | | | |
| No 02# | | | | | | | | | | | | | | | | | | |
| Cholest- | 0.19 | 0.24 | 0.39 | 0.75 | 0.72 | 0.69 | 0.29 | 0.41 | 0.39 | 0.45 | 0.51 | 0.51 | 0.44 | 0.47 | 0.63 | 0.22 | 0.22 | 0.4 |
| erol | * | * | * | * | * | * | * | * | * | * | * | * | * | * | * | * | * | * |
| Sphingo- | 0.19 | 0.24 | 0.34 | 0.74 | 0.73 | 0.63 | 0.39 | 0.38 | 0.37 | 0.49 | 0.52 | 0.6 | 0.82 | 0.78 | 0.63 | 0.22 | 0.18 | 0.28 |
| myelin | * | * | * | * | * | * | * | * | * | * | * | * | * | * | * | * | * | * |
| (d18:2, | | | | | | | | | | | | | | | | | | |
| C16:0)# | | | | | | | | | | | | | | | | | | |
| erythro- | 0.09 | 0.13 | 0.24 | 0.62 | 0.58 | 0.52 | 0.17 | 0.21 | 0.19 | 0.28 | 0.41 | 0.42 | 0.38 | 0.49 | 0.57 | 0.1 | 0.06 | 0.21 |
| Sphingo- | * | * | * | * | * | * | * | * | * | * | * | * | * | * | * | * | * | * |
| sine | | | | | | | | | | | | | | | | | | |
| (d18:1) | | | | | | | | | | | | | | | | | | |
| Ligno- | 0.2 | 0.21 | 0.43 | 0.7 | 0.68 | 0.67 | 0.28 | 0.39 | 0.19 | 0.49 | 0.63 | 0.63 | 0.45 | 0.39 | 0.64 | 0.14 | 0.17 | 0.35 |
| ceric | | * | * | * | * | * | * | * | * | * | * | * | * | * | * | * | * | * |
| acid | | | | | | | | | | | | | | | | | | |
| (C24:0) | | | | | | | | | | | | | | | | | | |
| Sphingo- | 0.2 | 0.37 | 0.44 | 0.68 | 0.71 | 0.7 | 0.34 | 0.28 | 0.27 | 0.41 | 0.63 | 0.6 | 0.52 | 0.62 | 0.65 | 0.23 | 0.22 | 0.35 |
| myelin | * | * | * | * | * | * | * | * | * | * | * | * | * | * | * | * | * | * |
| (d18:1, | | | | | | | | | | | | | | | | | | |
| C16:0) | | | | | | | | | | | | | | | | | | |

TABLE 15a
Markers for hormones (testosterone) in female rats; Significant up-regulation
changes (p-Value ≦ 0.2) are marked (*). For some metabolites (marked with #), additional
information are provided in table 19. All compounds were administered in high dose.

| | | | | | | | 17-alpha- | | |
|---|---|---|---|---|---|---|---|---|---|
| | Trenbolone | | | Mibolerone | | | Methyltestosterone | | |
| Metabolite | f7 | f14 | f28 | f7 | f14 | f28 | f7 | f14 | f28 |
| trans-4- | 1.31 * | 1.37 * | 1.66 * | 1.8 * | 2.44 * | 2 * | 1.4 * | 1.56 * | 1.61 * |
| Hydroxyproline | | | | | | | | | |
| Glycine | 1.18 * | 1.2 * | 1.37 * | 1.19 * | 1.22 * | 1.18 * | 1.28 * | 1.15 * | 1.29 * |
| 3-Hydroxybutyrate | 2.05 * | 1.73 * | 1.32 * | 1.36 * | 1.73 * | 1.06 | 1.5* | 2.03 * | 1.49 * |
| Glutamate | 1.18 | 1.13 * | 1.23 * | 1.66 * | 1.67 * | 1.73 * | 1.56 * | 1.43 * | 1.58 * |
| TAG (C16:0,C18:2)# | 1.83 * | 1.9 | 2.08 * | 0.87 | 6.82 * | 4.89 * | 4.63 * | 4.63 * | 5.09 * |
| TAG | 1.48 * | 2.26 * | 2.48 * | 1.22 | 8.88 * | 6.23 * | 8.68 * | 7.54 * | 9.21* |
| (C16:0,C18:1,C18:3)# | | | | | | | | | |
| TAG | 1.92 * | 2.39 * | 3.06 * | 0.99 | 5.98 * | 4.74 * | 6.36* | 5.29 * | 7.3* |
| (C18:3, C18:2, C18:1) | | | | | | | | | |
| TAG (C18:2,C18:2)# | 1.61 * | 2.21 * | 2.7 * | 0.9 | 6.69 * | 4.92 * | 4.67 * | 5.09 * | 8.15 * |
| TAG (C18:1,C18:2)# | 1.72 * | 1.97 * | 2.25 * | 0.77 | 8.37 * | 4.95 * | 5.83 * | 4.5 * | 5.32 * |
| TAG (C16:0,C16:1)# | 1.49 * | 1.41 | 1.28 * | 2.26 | 12.22 * | 5.83 * | 3.85 * | 2.59 * | 2.04 * |
| Threonic acid | 1.28 * | 1.76 * | 0.84 | 1.15 * | 1.18 * | 1.94 * | 1.65 * | 2.84 * | 2.29 * |
| DAG (C18:1,C18:2)# | 1.42 * | 1.56 | 1.79 * | 0.43 | 2.88 * | 2.27 * | 3.01 * | 3.17 * | 2.97 * |
| Phosphatidylcholine | 1.09 | 1.18 * | 1.17 * | 0.56 | 1.54 * | 1.32 * | 1.22 * | 1.15 * | 1.17* |
| (C16:0,C20:5)# | | | | | | | | | |
| TAG No 059# | 1.62 * | 2.14 * | 2.7 * | 1.08 * | 4.67 * | 3.5 * | 3.27 * | 4.08 * | 5.2 * |
| TAG (DAG- | 1.8 * | 2.67 * | 3.12 * | 0.95 | 7.86 * | 5.74 * | 5.82 * | 5.43 * | 6.9 * |
| Fragment)# | | | | | | | | | |

TABLE 15b
Markers for hormones (testosterone) in female rats; Significant down-regulation
changes (p-Value ≦ 0.2) are marked (*). For some metabolites (marked with #),
additional information are provided in table 19. All compounds were administered in high dose.

| | | | | | | | 17-alpha- | | |
|---|---|---|---|---|---|---|---|---|---|
| | Trenbolone | | | Mibolerone | | | Methyltestosterone | | |
| Metabolite | f7 | f14 | f28 | f7 | f14 | f28 | f7 | f14 | f28 |
| Lysine | 0.68 * | 0.62 * | 0.65 * | 0.68 * | 0.63 * | 0.57 * | 0.49 * | 0.49 * | 0.52 * |
| Phosphatidylcholine | 0.89 | 0.87 * | 0.93 | 0.18 * | 0.84 * | 0.74 * | 0.86 * | 0.78 * | 0.79 * |
| (C16:0,C22:6)# | | | | | | | | | |
| Normetanephrine | 0.83 * | 0.83 * | 0.75 * | 1.15 | 0.66 * | 0.78 * | 0.61 * | 0.65 * | 0.66 * |
| Phosphatidylcholine | 0.84 | 0.66 * | 0.81 * | 0.21 * | 0.93 | 0.86 | 0.78 * | 0.94 * | 0.81 * |
| (C18:0,C18:1)# | | | | | | | | | |
| Arginine | 0.76 * | 0.69 * | 0.78 * | 0.85 * | 0.98 * | 0.83 * | 0.69 * | 0.67 * | 0.59 * |
| Androstenedione | 0.3 * | 0.15 * | 0.23 * | 0.14 * | 0.11 * | 0.07 * | NA | NA | NA |
| Sphingomyelin | 0.51 * | 0.46 * | 0.64 * | 0.06 * | 1.26 | 0.63 * | 0.56 * | 0.52 * | 0.42 * |
| (d18:1,C23:0)# | | | | | | | | | |
| Campesterol | 0.53 * | 0.4 * | 0.9 | 0.16 | 0.89 | 0.54 * | 0.65 * | 0.62 * | 0.66 * |
| Lactate | 0.65 * | 0.56 * | 0.59 * | 0.62 * | 0.74 * | 0.73 * | 0.47 * | 0.34 * | 0.52 * |
| Pyruvate | 0.57 * | 0.61 * | 0.72 * | 0.75 * | 0.83 * | 0.65 * | 0.52 * | 0.35 * | 0.4 * |
| Phosphatidylcholine | 0.77 * | 0.75 * | 0.87 * | 0.2 * | 0.98 | 0.91 * | 0.69 * | 0.76 * | 0.68 * |
| (C18:0,C20:3)# | | | | | | | | | |
| Sphingomyelin | 0.62 * | 0.64 * | 0.74 * | 0.11 * | 1.33 * | 1.05 | 0.67 * | 0.6 * | 0.52 * |
| (d18:1,C24:0)# | | | | | | | | | |
| Metanephrine | 1 | 1.03 | 0.47 * | 0.62 * | 0.72 * | 0.66 * | 0.78 * | 0.66 * | 0.81 * |
| Ceramide | 0.5 * | 0.41 * | 0.69 * | 0.12 * | 2.02 * | 1.53 * | 0.79 * | 0.7 * | 0.52 * |
| (d18:1,C24:0) | | | | | | | | | |
| Progesterone | 0.34 * | 0.17 * | 0.3 * | 0.62 | 0.13 * | 0.06 * | NA | NA | NA |
| Phosphatidylcholine | 1.09 | 1.18 | 1.17 | 0.56 * | 1.54 | 1.32 | 1.22 | 1.15 | 1.17 |
| (C16:0,C20:5)# | | | | | | | | | |
| Urea | 0.74 * | 0.72 * | 0.54 * | 0.65 * | 0.48 * | 0.88 * | 0.72 * | 0.63 * | 0.8* |

TABLE 15c
Markers for Hormones (testosterone) in male rats; Significant up-regulation changes
(p-Value ≦ 0.2) are marked (*). For some metabolites (marked with #), additional
information are provided in table 19. All compounds were administered in high dose.

| | | | | | | | 17-alpha- | | |
|---|---|---|---|---|---|---|---|---|---|
| | Mibolerone | | | Trenbolone | | | Methyltestosterone | | |
| Metabolite | m7 | m14 | m28 | m7 | m14 | m28 | m7 | m14 | m28 |
| Mannose | 1.41 * | 1.08 * | 1.07 * | 1.35 * | 1.4 * | 1.31 * | 1.14 * | 1.03 | 1.2 * |
| Glycochenodeoxy- | 1.31 | 2.14 * | 5.73 * | NA | NA | NA | NA | NA | NA |
| cholic acid | | | | | | | | | |

TABLE 15d
Markers for Hormones (testosterone) in male rats; Significant down-regulation
changes (p-Value ≦ 0.2) are marked (*). For some metabolites (marked with #), additional
information are provided in table 19. All compounds were administered in high dose.

| | | | | | | | 17-alpha- | | |
|---|---|---|---|---|---|---|---|---|---|
| | Mibolerone | | | Trenbolone | | | Methyltestosterone | | |
| Metabolite | m7 | m14 | m28 | m7 | m14 | m28 | m7 | m14 | m28 |
| Kynurenic acid | 0.73 * | 0.5 | 0.54 * | 0.66 * | 0.8 * | 0.72 * | NA | NA | NA |
| Lysophosphatidyl- | 0.54 * | 0.87 | 0.85 * | 0.75 * | 0.83 * | 0.8 | 0.77 * | 0.88 * | 0.77 * |
| ethanolamine (C22:5)# | | | | | | | | | |
| Sphingomyelin | 0.1 * | 0.63 * | 1.01 | 0.37 * | 0.41 * | 0.54 * | 0.46 * | 0.47 * | 0.44 * |
| (d18:1,C24:0)# | | | | | | | | | |
| Homovanillic acid (HVA) | 0.5 * | 0.69 * | 0.6 * | 0.6 * | 0.5 * | 0.49 * | 0.74 * | 0.68 * | 0.92 |
| Phosphate, lipid fraction | 0.32 * | 0.62 * | 0.81 * | 0.47 * | 0.44 * | 0.66 * | 0.51 * | 0.64 * | 0.6 * |
| TAG No 02# | 0.34 * | 0.24 * | 0.23 * | 0.57 * | 0.74 * | 0.51 * | 0.41 * | 0.3 * | 0.34 * |
| Phosphatidylcholine | 0.19 * | 0.61 * | 1.1 | 0.62 * | 0.63 * | 0.87 | 0.78 | 0.69 * | 0.7 * |
| (C18:0,C18:1)# | | | | | | | | | |
| Lysophosphatidyl- | 0.53 * | 0.81 * | 0.91 * | 0.89 * | 0.82 * | 0.92 | 0.66 * | 0.78 * | 0.73 * |
| choline (C18:1)# | | | | | | | | | |
| Sphingomyelin | 0.17 * | 0.3 * | 0.33 * | 0.24 * | 0.26 * | 0.35 * | 0.4 | 0.31 * | 0.36 * |
| (d18:2,C18:0)# | | | | | | | | | |
| myo-Inositol, | 0.08 * | 0.29 * | 0.5 * | 0.24 * | 0.29 * | 0.54 * | 0.4 * | 0.49 * | 0.51 * |
| lipid fraction | | | | | | | | | |
| Glycerol phosphate, | 0.08 * | 0.35 * | 0.7 | 0.26 * | 0.24 * | 0.61 * | 0.28 * | 0.63 * | 0.47 * |
| lipid fraction# | | | | | | | | | |
| 3,4-Dihydroxyphenyl- | 0.83 * | 0.96 | 0.79 * | 1.08 | 0.75 * | 0.54 * | 0.6 * | 0.69 * | 0.74 |
| acetic acid (DOPAC) | | | | | | | | | |
| Sphingomyelin | 0.07 * | 0.53 * | 1.09 | 0.25 * | 0.33 * | 0.55 * | 0.49 * | 0.76 | 0.48 * |
| (d18:1,C23:0)# | | | | | | | | | |
| Taurocholic acid | 1.11 | 0.62 * | 0.75 * | NA | NA | NA | NA | NA | NA |
| Sphingomyelin | 0.12 * | 0.37 * | 0.51 * | 0.23 * | 0.22 * | 0.35 * | 0.34 * | 0.28 * | 0.27 * |
| (d18:1,C16:0) | | | | | | | | | |
| 18-Hydroxy-11- | 0.83 * | 0.93 | 0.63 * | 0.4 | 0.47 * | 0.52 * | 0.35 * | 0.94 | 0.55 * |
| deoxycorticosterone | | | | | | | | | |

TABLE 16
Markers for hormones (testosterone reduction) prostate in male rats; Significant
down-regulation changes (p-Value ≦ 0.2) are marked (*). For some metabolites (marked with #),
additional information are provided in table 19. All compounds were administered in high dose.

| | Trenbolone | | | Triticonazole | | |
|---|---|---|---|---|---|---|
| Metabolite | m7 | m14 | m28 | m7 | m14 | m28 |
| Phosphatidylcholine | 0.76 * | 0.74 * | 0.96 | 0.9 * | 0.86 * | 0.92 * |
| (C18:1,C18:2)# | | | | | | |
| Pantothenic acid | 0.65 * | 0.82 * | 0.77 * | 0.55 * | 0.93 | 0.85 * |
| Tyrosine | 0.93 * | 1 | 0.82 * | 0.78 * | 0.72 * | 0.82 * |
| 3,4-Dihydroxyphenylacetic | 1.08 | 0.75 * | 0.54 * | 0.61 * | 0.65 * | 0.64 * |
| acid (DOPAC) | | | | | | |
| Androstenedione | 0.09 * | 0.02 * | 0.02 * | 0.65 * | 1.37 | 0.42 * |
| Phosphatidylcholine No | 0.44 * | 0.42 * | 0.81 * | 0.68 * | 0.69 * | 0.67 * |
| 04# | | | | | | |
| Testosterone | 0.1 * | 0.06 * | 0.06 * | 0.55 * | 1.03 | 0.23 * |
| TAG (C18:1,C18:2)# | 0.77 * | 0.45 * | 1.26 | 0.4 * | 0.66 * | 0.52 * |
| Metanephrine | 0.86 | 0.48 * | 0.66 * | 0.91 | 0.75 * | 0.47 * |
| 3,4-Dihydroxyphenylglycol | 1.12 | 0.86 * | 0.72 * | 0.85 * | 0.81 * | 0.62 * |
| (DOPEG) | | | | | | |
| Glycerol, polar fraction | 0.66 * | 0.51 * | 0.61 * | 1.03 | 0.87 | 0.73 * |
| Ketoleucine | 0.93 | 0.85 * | 0.72 * | 0.99 | 0.7 | 0.75 * |
| Citrate | 0.89 * | 0.89 * | 1.03 | 0.88 * | 1.01 | 0.95 |
| Lactate | 0.68 * | 0.94 | 0.76 * | 0.82 * | 0.91 | 0.95 * |
| Histamine | 1.35 | 1.6 | 0.65 * | 0.85 | 0.74 * | 1.02 |

TABLE 17a
Markers for Pancreas endocrine modulation in female rats; Significant up-regulation
changes (p-Value ≦ 0.1) are marked (*). For some metabolites (marked with #), additional
information are provided in table 19. All compounds were administered in high dose.

| | | | | Fluoroglyco- | | | | | | Pioglitazone | | | Rosiglitazone | | | | | | Ziprasidone | | |
|---|---|---|---|---|---|---|---|---|---|---|---|---|---|---|---|---|---|---|---|---|---|
| Meta- | Clofibrate | | | fenethyl | | | Glipizide | | | hydrochloride | | | maleate | | | Streptozotocin | | | hydrochloride | | |
| bolite | f7 | f14 | f28 | f7 | f14 | f28 | f7 | f14 | f28 | f7 | f14 | f28 | f7 | f14 | f28 | f7 | f14 | f28 | f7 | f14 | f28 |
| Isopal- | 0.83 * | 0.84 | 1.53 * | 1.1 | 1.1 | 1.26 * | 0.75 | 1.42 * | 1.39 * | 1.22 * | 1.35 * | 1.2 | 1.46 * | 1.41 * | 1.52 * | 0.99 | 1.12 * | 1.3 * | 1.5 * | 1.41 | 1.1 |
| mitic | | | | | | | | | | | | | | | | | | | | | |
| acid | | | | | | | | | | | | | | | | | | | | | |
| (C16:0) | | | | | | | | | | | | | | | | | | | | | |

TABLE 17b
Markers for Pancreas endocrine modulation in female rats; Significant down-regulation changes
(p-Value ≦ 0.1) are marked (*). For some metabolites (marked with #), additional information
are provided in table 19. All compounds were administered in high dose.

| | | | | Fluorogly- | | | | | | Pioglitazone | | |
|---|---|---|---|---|---|---|---|---|---|---|---|---|
| Meta- | Clofibrate | | | cofenethyl | | | Glipizide | | | hydrochloride | | |
| bolite | f7 | f14 | f28 | f7 | f14 | f28 | f7 | f14 | f28 | f7 | f14 | f28 |
| Valine | 0.91 | 0.94 | 1.14 | 0.92 | 0.83 | 0.78 | 0.88 | 0.79 | 0.9 | 0.84 | 0.86 | 0.68 |
| | * | | | * | | * | * | * | * | * | * | * |
| Alanine | 0.63 | 0.72 | 0.84 | 0.74 | 1.06 | 1.1 | 0.59 | 0.6 | 0.67 | 0.82 | 0.71 | 0.61 |
| | * | * | * | * | | | * | * | * | * | * | * |
| Proline | 0.81 | 0.88 | 0.85 | 0.85 | 1 | 0.96 | 0.85 | 0.84 | 0.8 | 0.83 | 0.77 | 0.78 |
| | * | | * | * | | | * | * | * | * | * | * |
| Ketoleucine | 1.04 | 0.94 | 0.88 | 0.72 * | 0.87 | 0.82 | 0.86 | 0.83 | 0.89 | 0.81 | 0.74 | 0.81 |
| | | * | | | * | | * | * | * | * | * | * |
| Serine | 0.85 | 0.98 | 1.03 | 0.93 | 1.12 | 1 | 0.91 | 0.89 | 0.85 | 1.05 | 0.91 | 0.96 |
| | * | | | * | | | | * | * | | * | * |
| Lactate | 1.22 | 0.66 | 0.83 | 0.69 | 0.81 | 1.19 | 0.52 | 0.57 | 0.63 | 0.78 | 0.76 | 0.79 |
| | | * | | * | | | * | * | * | | * | * |
| Arginine | 0.74 | 0.93 | 0.92 | 0.91 | 1.06 | 1 | 0.8 | 0.74 | 0.83 | 0.9 | 0.85 | 0.84 |
| | * | | | * | | | * | * | * | | * | * |

| | |
|---|---|
| Rosiglitazone | Ziprasidone |

| Meta- | maleate | | | Streptozotocin | | | hydrochloride | | |
|---|---|---|---|---|---|---|---|---|---|
| bolite | f7 | f14 | f28 | f7 | f14 | f28 | f7 | f14 | f28 |
| Valine | 0.92 | 0.72 | 0.98 | 0.77 | 0.85 | 0.98 | 1.13 | 0.84 | 0.97 |
| | | * | | | * | | | * | |
| Alanine | 0.83 | 0.81 | 0.92 | 0.79 | 0.91 | 0.87 | 0.83 | 0.82 | 0.76 |
| | * | * | * | * | * | * | * | * | * |
| Proline | 0.91 | 0.81 | 1.03 | 0.81 | 0.83 | 0.9 | 1.1 | 0.94 | 0.84 |
| | | * | | * | * | | | | * |
| Ketoleucine | 0.72 | 0.71 | 0.67 | 0.85 | 0.9 | 0.87 | 0.9 | 1.01 | 0.95 |
| | * | * | * | * | * | * | * | | * |
| Serine | 1 | 0.91 | 1.06 | 0.88 | 0.83 | 0.98 | 1.12 | 1.04 | 0.83 |
| | | * | | * | * | | * | | * |
| Lactate | 0.56 | 1.24 | 0.65 | 0.77 | 0.83 | 0.57 | 0.6 | 0.66 | 0.62 |
| | * | | * | * | * | * | * | | * |
| Arginine | 0.85 | 0.73 | 0.93 | 0.78 | 0.88 | 1.03 | 1.13 | 1.11 | 0.93 |
| | | * | * | * | * | | * | | * |

TABLE 18
Compounds and dosing (CMC = Carboxymethyl cellulose)

| Compound | Dose | Synonym | CAS no | Dosage administered | Details |
|---|---|---|---|---|---|
| 17-alpha-Ethynylestradiol (*) | low | 17-ethynyl-13-methyl- | 57-63-6 | 50 μg/kg body weight | in corn oil; |
| | | 7,8,9,11,12,13,14,15,16,17- | | by gavage | administration volume: |
| | | decahydro-6H- | | | 5 ml/kg body weight |
| | | cyclopenta[a]phenanthrene-3,17-diol | | | |
| 17-alpha-Ethynylestradiol | high | 17-ethynyl-13-methyl- | 57-63-6 | 200 mg/kg by gavage | in corn oil; |
| | | 7,8,9,11,12,13,14,15,16,17- | | | administration volume: |
| | | decahydro-6H- | | | 5 ml/kg body weight |
| | | cyclopenta[a]phenanthrene-3,17-diol | | | |
| 17-alpha-Methyltestosterone | high | na | 58-18-4 | 80 mg/kg body weight | in corn oil; |
| | | | | by gavage | administration volume: |
| | | | | | 5 ml/kg body weight |
| ACTH | high | Tetracosactide | 16960-16-0 | 0.05 mg/kg bw | in 0.9% NaCl; |
| | | | | subcutaneous | administration volume: |
| | | | | | 1 ml/kg bw; daily |
| Bromocriptine Mesylate | high | na | 22260-51-1 | 10 mg/kg body weight | in aqua bidest. |
| | | | | by gavage | containing 0.5% CMC |
| | | | | | (Tylose CB30000), |
| | | | | | administration volume: |
| | | | | | 10 ml/kg body weight |
| Buserelin acetate s.c. | high | na | 68630-75-1 | 25 μg/kg body weight | in 0.9% NaCl; |
| | | | | subcutaneous | administration volume: |
| | | | | | 1 ml/kg bw |
| Cabergoline | high | Dostinex | 81409-90-7 | 5 mg/kg body weight by | In drinking water |
| | | | | gavage (days 0 to 2), | containing 1% CMC; |
| | | | | 2.5 mg/kg body weight | administration volume: |
| | | | | by gavage (from day 3 | 10 ml/kg bw. |
| | | | | on) | |
| Clofibrate | high | Ethyl 2-(4-chlorophenoxy)-2-methyl- | 637-07-0 | 200 mg/kg body weight | in corn oil, |
| | | propanoate | | by gavage | administration volume: |
| | | | | | 5 ml/kg bw |
| Cyproteron Acetate | high | 6-chloro-1-beta,2-beta-dihydro-17- | 427-51-0 | 250 mg/kg body weight | in drinking water |
| | | hydroxy-3′H-cyclopropa[1,2]pregna- | | by gavage | containing 0.5% CMC |
| | | 1,4,6-triene-3,20-dione 17-acetate | | | (Tylose CB30000), |
| | | | | | administration volume: |
| | | | | | 10 ml/kg bw |
| Dexamethasone | high | 9alpha-Fluoro-11beta,17alpha,21- | 50-02-2 | 30 μg/kg body weight | in drinking water |
| | | trihydroxy-16alpha-methylpregn-1,4- | | by gavage | containing 0.5% CMC |
| | | diene-3,20-dione | | | (Tylose CB30000), |
| | | | | | administration volume: |
| | | | | | 10 ml/kg bw |
| Diethanolamine | high | 2,2′-Dihydroxydiethylamine | 111-42-2 | 50 (males) and 100 | in aqua bidest., |
| | | | | (females) mg/kg body | administration volume: |
| | | | | weight by gavage | 10 ml/kg body weight |
| Diethylstilboestrol dipropionate | high | (E)-4,4′-(1,2-Diethyl-1,2- | 130-80-3 | 5 μg/kg body weight | in corn oil; |
| | | ethenediyl)bisphenol dipropionate | | by gavage | administration volume: |
| | | | | | 5 ml/kg body weight |
| Epoxiconazole | high | na | 106325-08-0 | 2,000 ppm in the diet | mixture in the diet |
| Epoxlconazole (*) | low | na | 106325-08-0 | 500 ppm in the diet | mixture in the diet |
| Fenarimol | low | na | 60168-88-9 | 250 ppm in the diet | mixture in the diet |
| Fluoroglycofen-ethyl | high | O-[5-(2-chloro-a,a,a-trifluoro-p- | 77501-90-7 | 3,200 ppm in the diet | mixture in the diet |
| | | tolyloxy)-2-nitrobenzoyl]glycolic acid | | | |
| Fluoxetine hydrochloride | high | na | 59333-67-4 | 20 mg/kg body weight | in drinking water |
| | | | | by gavage | containing 0.5% CMC |
| | | | | | (Tylose CB30000), |
| | | | | | administration volume: |
| | | | | | 10 ml/kg bw |
| Flutamide | high | na | 13311-84-7 | 100 mg/kg body weight | in corn oil, |
| | | | | by gavage | administration volume: |
| | | | | | 5 ml/kg body weight |
| Formestane | low | na | 566-48-3 | 100 mg/kg body weight | in 0.9% NaCl; |
| | | | | i.p. | administration volume: |
| | | | | | 3 ml/kg bw; twice a week |
| Genistein | low | 5,7-dihydroxy-3-(4-hydroxyphenyl)- | 446-72-0 | 300 mg/kg bw by | in drinking water |
| | | chromen-4-one | | gavage | containing 1% CMC; |
| | | | | | administration volume: |
| | | | | | 10 ml/kg bw |
| Glipizide | high | na | 29094-61-9 | 75 mg/kg body weight | in drinking water |
| | | | | by gavage | containing 0.5% CMC |
| | | | | | (Tylose CB30000), |
| | | | | | administration volume: |
| | | | | | 10 ml/kg bw |
| Ifosfamide | high | N,3-Bis(2-Chloroethyl)tetrahydro-2H- | 3778-73-2 | 40 mg/kg bw/d i.p | in saline; |
| | | 1,3,2-oxazaphosphorin-2-amine-2- | | | administration volume: |
| | | oxide | | | 3 ml/kg bw. |
| Iprodione | high | 3-(3,5-dichlorophenyl)-N-isopropyl- | 36734-19-7 | 2,500 ppm in the diet | mixture in the diet |
| | | 2,4-dioxoimidazolidine-1-carboxamide | | | |
| Leuprolide acetate salt | high | na | 74381-53-6 | 15 μg/kg body weight | in 0.9% NaCl; |
| | | | | s.c. | administration volume: |
| | | | | | 1 ml/kg bw |
| Medroxyprogesterone acetat | high | 17-alpha-hydroxy-6-alpha-methyl- | 71-58-9 | 334 mg/kg bw/d by | in 1% CMC |
| | | progesteronacetate | | gavage | administration volume: |
| | | | | | 10 ml/kg bw. |
| Metazachlor | high | N-(2,6-dimethylphenyl)-N-(pyrazol-1- | 67129-08-2 | 20,000 ppm in the diet | mixture in the diet |
| | | ylmethyl)chloroacetamid | | | |
| Methimazole | high | na | 60-56-0 | 100 mg/kg body weight | in drinking water, |
| | | | | by gavage | administration volume: |
| | | | | | 10 ml/kg bw |
| Mibolerone | high | na | 3704-09-4 | 10 mg/kg bw by gavage | in drinking water |
| | | | | | containing 1% CMC; |
| | | | | | administration volume: |
| | | | | | 10 ml/kg bw |
| Mifepristone | high | (11b,17b)-11-[4- | 84371-65-3 | 0.3 mg/kg body weight | in drinking water |
| | | (Dimethylamino)phenyl]-17-hydroxy- | | by gavage | containing 1% CMC; |
| | | 17-(1-propynyl)estra-4,9-dien-3-one | | | administration volume |
| | | | | | 10 ml/kg |
| Naphthylisothiocyanate | high | 1-isothiocyanate-naphthalene | 551-06-4 | 2500 ppm in the diet | mixture in the diet |
| | | | | (days 0 to 2), 1000 ppm | |
| | | | | in the diet (from day | |
| | | | | 3 on) | |
| Norethindrone acetate | high | na | 51-98-9 | 1500 ppm in the diet | mixture in the diet |
| Pioglitazone hydrochloride | high | Actos | 112529-15-4 | 100 mg/kg body weight | in drinking water |
| | | | | by gavage | containing 0.5% CMC |
| | | | | | (Tylose CB30000), |
| | | | | | administration volume: |
| | | | | | 10 ml/kg bw |
| Raloxifene Hydrochloride | high | Evista | 82640-04-8 | 600 mg/kg body weight | in drinking water |
| | | | | by gavage | containing 0.5% CMC |
| | | | | | (Tylose CB30000), |
| | | | | | administration volume: |
| | | | | | 10 ml/kg bw |
| Risperidone | high | Risperdal | 106266-06-2 | 20 mg/kg body weight | in drinking water |
| | | | | by gavage | containing 0.5% CMC |
| | | | | | (Tylose CB30000), |
| | | | | | administration volume: |
| | | | | | 10 ml/kg bw |
| Rosiglitazone maleate | high | na | 155141-29-0 | 5 mg/kg body weight | in drinking water |
| | | | | by gavage | containing 0.5% CMC |
| | | | | | (Tylose CB30000), |
| | | | | | administration volume: |
| | | | | | 10 ml/kg bw |
| Streptozotocin | high | N-[Methylnitrosocarbomyl]-D- | 18883-66-4 | 30 mg/kg body weight | in aqua bidest., |
| | | glucosamide | | by gavage | administration volume: |
| | | | | | 10 ml/kg body weight |
| Tamoxifen | high | (Z)-2-[4-(1,2-diphenylbut-1- | 10540-29-1 | 200 μg/kg body weight | in corn oil; |
| | | enyl)phenoxy]-N,N-dimethyl- | | by gavage | administration volume: |
| | | ethanamine | | | 5 ml/kg body weight |
| Trenbolone | high | 17beta-Hydroxyestra-4,9,11-trien-3- | 10161-33-8 | 40 mg/kg body weight | in corn oil; |
| | | one | | by gavage | administration volume: |
| | | | | | 5 ml/kg bw |
| Triticonazole (*) | low | (+−)-(E)-5-(4-Chlorobenzylidene)-2,2- | 131983-72-7 | 5,000 ppm in the diet | mixture in the diet |
| | | dimethyl-1-(1H-1,2,4-triazol-1- | | | |
| | | ylmethyl)cyclopentanol | | | |
| Triticonazole | high | (+−)-(E)-5-(4-Chlorobenzylidene)-2,2- | 131983-72-7 | 20,000 ppm in the diet | mixture in the diet |
| | | dimethyl-1-(1H-1,2,4-triazol-1- | | (days 0 to 13), 15,000 | |
| | | ylmethyl)cyclopentanol | | ppm in the diet (from | |
| | | | | day 14 on) | |
| Vinclozolin | high | 3-(3,5-Dichlorphenyl)-5-methyl-5- | 50471-44-8 | 3,000 ppm in the diet | mixture in the diet |
| | | vinyl-1,3-oxazolidin-2,4-dion | | | |
| Ziprasidone hydrochloride | high | na | 122883-93-6 | 150 mg/kg body weight | in drinking water |
| | | | | by gavage | containing 0.5% CMC |
| | | | | | (Tylose CB30000), |
| | | | | | administration volume: |
| | | | | | 10 ml/kg bw |

(*): low dose for [special patterns] only:
Epoxiconazole (*): low dose for Adrenals aromatase inhibitors (M LD group Str 24 01 08 W)
17-alpha-Ethynylestradiol (*): low dose for Hormone_estronenic (LD_F_group_wm_Welch)
Triticonazole (*): low dose for Adrenals aromatase inhibitors (M LD group Str 24 01 08 W)

TABLE 19
Chemical/physical properties of selected analytes. These biomarkers are characterized herein by chemical and physical properties.

| Metabolite | Fragmentation pattern (GC-MS) and description |
|---|---|
| 3-O-Methylsphingosine (d18:1) | 3-O-Methylsphingosine (d18:1) exhibits the following characteristic ionic fragments when detected |
| | with GC/MS, applying electron impact (EI) ionization mass spectrometry, after acidic |
| | methanolysis and derivatisation with 2% O-methylhydroxylamine-hydrochlorid in pyridine and |
| | subsequently with N-methyl-N-trimethylsilyltrifluoracetamid: MS (EI, 70 eV): m/z (%): 204 |
| | (100), 73 (18), 205 (16), 206 (7), 354 (4), 442 (1). |
| 5-O-Methylsphingosine (d18:1) | 5-O-Methylsphingosine (d18:1) exhibits the following characteristic ionic fragments when detected |
| | with GC/MS, applying electron impact (EI) ionization mass spectrometry, after acidic |
| | methanolysis and derivatisation with 2% O-methylhydroxylamine-hydrochlorid in pyridine and |
| | subsequently with N-methyl-N-trimethylsilyltrifluoracetamid: MS (EI, 70 eV): m/z (%): 250 |
| | (100), 73 (34), 251 (19), 354 (14), 355 (4), 442 (1). |
| Cholesterolester No 01 | Metabolite belongs to the class of cholesterolesters. It exhibits the following characteristic |
| | ionic species when detected with LC/MS, applying electro-spray ionization (ESI) mass spectrometry: |
| | mass-to-charge ratio (m/z) of the positively charged ionic species is 369.2 (+/−0.5). |
| Choline plasmalogen No 01 | Metabolite belongs to the class of choline plasmalogens. It exhibits the following characteristic |
| | ionic species when detected with LC/MS, applying electro-spray ionization (ESI) mass spectrometry: |
| | mass-to-charge ratio (m/z) of the positively charged ionic species is 772.6 (+/−0.5). |
| Choline plasmalogen No 02 | Metabolite belongs to the class of choline plasmalogens. It exhibits the following characteristic |
| | ionic species when detected with LC/MS, applying electro-spray ionization (ESI) mass spectrometry: |
| | mass-to-charge ratio (m/z) of the positively charged ionic species is 767 (+/−0.5). |
| Choline plasmalogen No 03 | Metabolite belongs to the class of choline plasmalogens. It exhibits the following characteristic |
| | ionic species when detected with LC/MS, applying electro-spray ionization (ESI) mass spectrometry: |
| | mass-to-charge ratio (m/z) of the positively charged ionic species is 768.8 (+/−0.5). |
| DAG (C18:1, C18:2) | DAG (C18:1, C18:2) represents the sum parameter of diacylglycerols containing the combination |
| | of a C18:1 fatty acid unit and a C18:2 fatty acid unit. The mass-to-charge ratio (m/z) of the |
| | ionised species is 641.6 Da (+/−0.5 Da). |
| Eicosaenoic acid (C20:1) No | Eicosaenoic acid (C20:1) exhibits the following characteristic ionic fragments when detected |
| 02 | with GC/MS, applying electron impact (EI) ionization mass spectrometry, after acidic meth- |
| | anolysis and derivatisation with 2% O-methylhydroxylamine-hydrochlorid in pyridine and sub- |
| | sequently with N-methyl-N-trimethylsilyltrifluoracetamid: MS (EI, 70 eV): m/z (%): 55 (100), 69 |
| | (75), 41 (57), 83 (54), 74 (53), 97 (45), 110 (20), 292 (13), 293 (13), 124 (12), 250 (9), 152 (8), |
| | 138 (8), 208 (7), 324(2). |
| Glycerol phosphate, lipid frac- | Glycerol phosphate, lipid fraction represents the sum parameter of metabolites containing a |
| tion | glycerol-2-phosphate or a glycerol-3-phosphate moiety and being present in the lipid fraction |
| | after extraction and separation of the extract into a polar and a lipid fraction. |
| Lysophosphatidylcholine | Lysophosphatidylcholine (C17:0) represents the sum parameter of lysoglycerophosphorylcholines |
| (C17:0) | containing a C17:0 fatty acid unit. If detected with LC/MS, applying electro-spray ionization |
| | (ESI) mass spectrometry, the mass-to-charge ratio (m/z) of the positively charged ionic |
| | species is 510.4 Da (+/−0.5 Da). |
| Lysophosphatidylcholine | Lysophosphatidylcholine (C18:0) represents the sum parameter of lysoglycerophosphorylcholines |
| (C18:0) | containing a C18:0 fatty acid unit. If detected with LC/MS, applying electro-spray ionization |
| | (ESI) mass spectrometry, the mass-to-charge ratio (m/z) of the positively charged ionic |
| | species is 546.6 Da (+/−0.5 Da). |
| Lysophosphatidylcholine | Lysophosphatidylcholine (C18:1) represents the sum parameter of lysoglycerophosphorylcholines |
| (C18:1) | containing a C18:1 fatty acid unit. If detected with LC/MS, applying electro-spray ionization |
| | (ESI) mass spectrometry, the mass-to-charge ratio (m/z) of the positively charged ionic |
| | species is 522.2 Da (+/−0.5 Da). |
| Lysophosphatidylcholine | Lysophosphatidylcholine (C18:2) represents the sum parameter of lysoglycerophosphorylcholines |
| (C18:2) | containing a C18:2 fatty acid unit. If detected with LC/MS, applying electro-spray ionization |
| | (ESI) mass spectrometry, the mass-to-charge ratio (m/z) of the positively charged ionic |
| | species is 542.4 Da (+/−0.5 Da). |
| Lysophosphatidylcholine | Lysophosphatidylcholine (C20:4) represents the sum parameter of lysoglycerophosphorylcholines |
| (C20:4) | containing a C20:4 fatty acid unit. If detected with LC/MS, applying electro-spray ionization |
| | (ESI) mass spectrometry, the mass-to-charge ratio (m/z) of the positively charged ionic |
| | species is 544.4 Da (+/−0.5 Da). |
| Lysophosphatidylethanolamine | Lysophosphatidylethanolamine (C22:5) exhibits the following characteristic ionic species |
| (C22:5) | when detected with LC/MS, applying electro-spray ionization (ESI) mass spectrometry: |
| | mass-to-charge ratio (m/z) of the positively charged ionic species is 528.2 (+/−0.5). |
| Phosphatidylcholine | Phosphatidylcholine (C16:0/C16:0) represents the sum parameter of glycerophosphorylcholines |
| (C16:0, C16:0) | containing either the combination of of two C16:0 fatty acid units. The mass-to-charge |
| | ratio (m/z) of the ionised species is 734.8 Da (+/−0.5 Da). |
| Phosphatidylcholine | Phosphatidylcholine (C16:0, C20:5) exhibits the following characteristic ionic species when |
| (C16:0, C20:5) | detected with LC/MS, applying electro-spray ionization (ESI) mass spectrometry: |
| | mass-to-charge ratio (m/z) of the positively charged ionic species is 780.8 (+/−0.5). |
| Phosphatidylcholine | Phosphatidylcholine (C16:1, C18:2) represents the sum parameter of glycerophosphorylcholines |
| (C16:1, C18:2) | containing the combination of a C16:1 fatty acid unit and a C18:2 fatty acid unit. |
| | If detected with LC/MS, applying electro-spray ionization (ESI) mass spectrometry, |
| | the mass-to-charge ratio (m/z) of the positively charged ionic species is 756.8 Da (+/−0.5 Da). |
| Phosphatidylcholine | Phosphatidylcholine (C18:0, C18:1) represents the sum parameter of glycerophosphorylcholines |
| (C18:0, C18:1) | containing the combination of a C18:0 fatty acid unit and a C18:1 fatty acid unit. If detected |
| | with LC/MS, applying electro-spray ionization (ESI) mass spectrometry, the mass-to-charge |
| | ratio (m/z) of the positively charged ionic species is 788.6 Da (+/−0.5 Da). |
| Phosphatidylcholine | Phosphatidylcholine (C18:0, C18:2) represents the sum parameter of glycerophosphorylcholines |
| (C18:0, C18:2) | containing the combination of a C18:0 fatty acid unit and a C18:2 fatty acid unit. If detected |
| | with LC/MS, applying electro-spray ionization (ESI) mass spectrometry, the mass-to-charge |
| | ratio (m/z) of the positively charged ionic species is 786.6 Da (+/−0.5 Da). |
| Phosphatidylcholine | Phosphatidylcholine (C18:0, C20:3) exhibits the following characteristic ionic species when |
| (C18:0, C20:3) | detected with LC/MS, applying electro-spray ionization (ESI) mass spectrometry: |
| | mass-to-charge ratio (m/z) of the positively charged ionic species is 812.6 (+/−0.5). |
| Phosphatidylcholine | Phosphatidylcholine (C18:0, C20:4) represents the sum parameter of glycerophosphorylcholines |
| (C18:0, C20:4) | containing the combination of a C18:0 fatty acid unit and a C20:4 fatty acid unit. If detected |
| | with LC/MS, applying electro-spray ionization (ESI) mass spectrometry, the mass-to-charge |
| | ratio (m/z) of the positively charged ionic species is 810.8 Da (+/−0.5 Da). |
| Phosphatidylcholine | Phosphatidylcholine (C18:0, C22:6) represents the sum parameter of glycerophosphorylcholines |
| (C18:0, C22:6) | containing the combination of a C18:0 fatty acid unit and a C22:6 fatty acid unit. If detected |
| | with LC/MS, applying electro-spray ionization (ESI) mass spectrometry, the mass-to-charge |
| | ratio (m/z) of the positively charged ionic species is 834.8 Da (+/−0.5 Da). |
| Phosphatidylcholine | Phosphatidylcholine (C16:0/C20:3 C18:1/C18:2) represents the sum parameter of glycerophosphorylcholines |
| (C18:1, C18:2) | containing the combination of a C18:1 fatty acid unit and a C18:2 fatty acid unit. |
| | The mass-to-charge ratio (m/z) of the ionised species is 784.6 Da (+/−0.5 Da). |
| Phosphatidylcholine | Phosphatidylcholine (C16:0/C22:6) represents the sum parameter of glycerophosphorylcholines |
| (C16:0/C22:6) | containing either the combination of a C16:0 fatty acid unit and a C22:6 fatty acid unit or |
| | the combination of a C18:2 fatty acid unit and a C20:4 fatty acid unit. The mass-to-charge |
| | ratio (m/z) of the ionised species is 806.6 Da (+/−0.5 Da). |
| Phosphatidylcholine No 02 | Metabolite belongs to the class of glycerophosphocholines. It exhibits the following characteristic |
| | ionic species when detected with LC/MS, applying electro-spray ionization (ESI) mass spectrometry: |
| | mass-to-charge ratio (m/z) of the positively charged ionic species is 808.4 (+/−0.5). |
| Phosphatidylcholine No 04 | Metabolite belongs to the class of glycerophosphocholines. It exhibits the following characteristic |
| | ionic species when detected with LC/MS, applying electro-spray ionization (ESI) mass spectrometry: |
| | mass-to-charge ratio (m/z) of the positively charged ionic species is 796.8 (+/−0.5). |
| Sphingomyelin (d18:1, C23:0) | Sphingomyelin (d18:1, C23:0) exhibits the following characteristic ionic species when detected |
| | with LC/MS, applying electro-spray ionization (ESI) mass spectrometry: mass-to-charge ratio |
| | (m/z) of the positively charged ionic species is 801.8 (+/−0.5). |
| Sphingomyelin (d18:1, C24:0) | Sphingomyelin (d18:1, C24:0) represents the sum parameter of sphingomyelins containing |
| | the combination of a d18:1 long-chain base unit and a C24:0 fatty acid unit. If detected with |
| | LC/MS, applying electro-spray ionization (ESI) mass spectrometry, the mass-to-charge ratio |
| | (m/z) of the positively charged ionic species is 815.8 Da (+/−0.5 Da). |
| Sphingomyelin (d18:2, C16:0) | Sphingomyelin (d18:2, C16:0) exhibits the following characteristic ionic species when detected |
| | with LC/MS, applying electro-spray ionization (ESI) mass spectrometry: mass-to-charge ratio |
| | (m/z) of the positively charged ionic species is 723.6 (+/−0.5). |
| Sphingomyelin (d18:2, C18:0) | Sphingomyelin (d18:2, C18:0) exhibits the following characteristic ionic species when detected |
| | with LC/MS, applying electro-spray ionization (ESI) mass spectrometry: mass-to-charge ratio |
| | (m/z) of the positively charged ionic species is 729.8 (+/−0.5). |
| TAG (C16:0, C16:1) | Metabolite represents the sum of triacylglycerides containing the combination of a C16:0 fatty |
| | acid unit and a C16:1 fatty acid unit. It exhibits the following characteristic ionic species when |
| | detected with LC/MS, applying electro-spray ionization (ESI) mass spectrometry: mass-to-charge |
| | ratio (m/z) of the positively charged ionic species is 549.6 (+/−0.5). |
| TAG (C16:0, C18:1, C18:3) | TAG (C16:0, C18:1, C18:3) exhibits the following characteristic ionic species when detected |
| | with LC/MS, applying electro-spray ionization (ESI) mass spectrometry: mass-to-charge ratio |
| | (m/z) of the positively charged ionic species is 855.6 (+/−0.5). |
| TAG (C16:0, C18:2) | Metabolite represents the sum of triacylglycerides containing the combination of a C16:0 fatty |
| | acid unit and a C18:2 fatty acid unit. It exhibits the following characteristic ionic species when |
| | detected with LC/MS, applying electro-spray ionization (ESI) mass spectrometry: mass-to-charge |
| | ratio (m/z) of the positively charged ionic species is 575.6 (+/−0.5). |
| TAG (C18:1, C18:2) | Metabolite represents the sum of triacylglycerides containing the combination of a C18:1 fatty |
| | acid unit and a C18:2 fatty acid unit. It exhibits the following characteristic ionic species when |
| | detected with LC/MS, applying electro-spray ionization (ESI) mass spectrometry: mass-to-charge |
| | ratio (m/z) of the positively charged ionic species is 601.6 (+/−0.5). |
| TAG (C18:2, C18:2) | Metabolite represents the sum of triacylglycerides containing the combination of a C18:2 fatty |
| | acid unit and a C18:2 fatty acid unit. It exhibits the following characteristic ionic species when |
| | detected with LC/MS, applying electro-spray ionization (ESI) mass spectrometry: mass-to-charge |
| | ratio (m/z) of the positively charged ionic species is 599.6 (+/−0.5). |
| TAG (C18:2, C18:3) | Metabolite represents the sum of triacylglycerides containing the combination of a C18:2 fatty |
| | acid unit and a C18:3 fatty acid unit. It exhibits the following characteristic ionic species when |
| | detected with LC/MS, applying electro-spray ionization (ESI) mass spectrometry: mass-to-charge |
| | ratio (m/z) of the positively charged ionic species is 597.6 (+/−0.5). |
| TAG (DAG-Fragment) | Metabolite belongs to the class of triacylglycerides. It exhibits the following characteristic ionic |
| | species when detected with LC/MS, applying electro-spray ionization (ESI) mass spectrometry: |
| | mass-to-charge ratio (m/z) of the positively charged ionic species is 600.6 (+/−0.5). |
| TAG No 01 | Metabolite belongs to the class of triacylglycerides. It exhibits the following characteristic ionic |
| | species when detected with LC/MS, applying electro-spray ionization (ESI) mass spectrometry: |
| | mass-to-charge ratio (m/z) of the positively charged ionic species is 547.6 (+/−0.5). |
| TAG No 02 | Metabolite belongs to the class of triacylglycerides. It exhibits the following characteristic ionic |
| | species when detected with LC/MS, applying electro-spray ionization (ESI) mass spectrometry: |
| | mass-to-charge ratio (m/z) of the positively charged ionic species is 695.6 (+/−0.5). |
| TAG (C18:3, C18:2, C18:1)# | TAG (C18:3, C18:2, C18:1) represents the sum parameter of triacylglycerides containing either |
| | the combination of a C18:3 fatty acid, a C18:2 fatty acid unit and a C18:1 fatty acid unit or the |
| | combination of a C20:4 fatty acid, a C18:2 fatty acid unit and a C16:0 fatty acid unit or the |
| | combination of a C20:5 fatty acid, a C18:1 fatty acid unit and a C16:0 fatty acid unit. |
| | Metabolite(s) belong to the class of triacylglycerides. It exhibits the following characteristic |
| | ionic species when detected with LC/MS, applying electro-spray ionization (ESI) mass spectrometry: |
| | mass-to-charge ratio (m/z) of the positively charged ionic species is 879.6 (+/−0.5). |
| TAG No 059 | Metabolite belongs to the class of triacylglycerides. It exhibits the following characteristic ionic |
| | species when detected with LC/MS, applying electro-spray ionization (ESI) mass spectrometry: |
| | mass-to-charge ratio (m/z) of the positively charged ionic species is 904 (+/−0.5). |
| TAG No 07 | Metabolite belongs to the class of triacylglycerides. It exhibits the following characteristic ionic |
| | species when detected with LC/MS, applying electro-spray ionization (ESI) mass spectrometry: |
| | mass-to-charge ratio (m/z) of the positively charged ionic species is 853.6 (+/−0.5). |

1. A method for diagnosing an endocrine disease or disorder comprising:
(a) determining the amount of at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, or 9 in a test sample of a subject suspected to suffer from an endocrine disease or disorder, and
(b) comparing the amounts determined in step (a) to a reference, whereby an endocrine disease or disorder is to be diagnosed.

2. The method of claim 1, wherein said subject has been brought into contact with a compound suspected to be capable of inducing an endocrine disease or disorder.

3. A method of determining whether a compound is capable of inducing an endocrine disease or disorder in a subject comprising:
(a) determining in a sample of a subject which has been brought into contact with a compound suspected to be capable of inducing an endocrine disease or disorder the amount of at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, or 9; and
(b) comparing the amounts determined in step (a) to a reference, whereby the capability of the compound to induce an endocrine disease or disorder is determined.

4. The method of claim 2, wherein said compound is at least one compound selected from the group consisting of: 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, and Ziprasidone hydrochloride.

5. The method of claim 1, wherein said reference is derived from (i) a subject or group of subjects which suffers from an endocrine disease or disorder or (ii) a subject or group of subjects which has been brought into contact with at least one compound selected from the group consisting of: 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, and Ziprasidone hydrochloride.

6. The method of claim 5, wherein essentially identical amounts for the biomarkers in the test sample and the reference are indicative for an endocrine disease or disorder.

7. The method of claim 1, wherein said reference is derived from (i) a subject or group of subjects known to not suffer from an endocrine disease or disorder or (ii) a subject or group of subjects which has not been brought into contact with at least one compound selected from the group consisting of: 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, and Ziprasidone hydrochloride.

8. The method of claim 1, wherein said reference is a calculated reference for the biomarkers for a population of subjects.

9. The method of claim 7, wherein amounts for the biomarkers which differ in the test sample in comparison to the reference are indicative for an endocrine disease or disorder.

10. A method of identifying a substance for treating an endocrine disease or disorder comprising the steps of:
(a) determining in a sample of a subject suffering from an endocrine disease or disorder which has been brought into contact with a candidate substance suspected to be capable of treating an endocrine disease or disorder the amount of at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, or 9; and
(b) comparing the amounts determined in step (a) to a reference, whereby a substance capable of treating an endocrine disease or disorder is to be identified.

11. The method of claim 10, wherein said reference is derived from (i) a subject or group of subjects which suffers from an endocrine disease or disorder or (ii) a subject or group of subjects which has been brought into contact with at least one compound selected from the group consisting of: 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, and Ziprasidone hydrochloride.

12. The method of claim 11, wherein amounts for the biomarkers which differ in the test sample and the reference are indicative for a substance capable of treating an endocrine disease or disorder.

13. The method of claim 10, wherein said reference is derived from (i) a subject or group of subjects known to not suffer from an endocrine disease or disorder or (ii) a subject or group of subjects which has not been brought into contact with at least one compound selected from the group consisting of: 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, and Ziprasidone hydrochloride.

14. The method of claim 10, wherein said reference is a calculated reference for the biomarkers in a population of subjects.

15. The method of claim 13, wherein essentially identical amounts for the biomarkers in the test sample and the reference are indicative for a substance capable of treating an endocrine disease or disorder.

16. (canceled)

17. A device for diagnosing an endocrine disease or disorder in a sample of a subject suspected to suffer therefrom comprising:
(a) an analyzing unit comprising a detection agent for at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, or 9 which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby an endocrine disease or disorder is diagnosed.

18. The device of claim 17, wherein said stored reference is a reference derived from a subject or a group of subjects known to suffer from an endocrine disease or disorder or a subject or group of subjects which has been brought into contact with at least one compound selected from the group consisting of 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, and Ziprasidone hydrochloride and said data processor executes instructions for comparing the amount of the at least one biomarker determined by the analyzing unit to the stored reference, wherein an essentially identical amount of the at least one biomarker in the test sample in comparison to the reference is indicative for the presence of an endocrine disease or disorder or wherein an amount of the at least one biomarker in the test sample which differs in comparison to the reference is indicative for the absence of an endocrine disease or disorder.

19. The device of claim 17, wherein said stored reference is a reference derived from a subject or a group of subjects known to not suffer from an endocrine disease or disorder or a subject or group of subjects which has not been brought into contact with at least one compound selected from the group consisting of 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, and Ziprasidone hydrochloride and said data processor executes instructions for comparing the amount of the at least one biomarker determined by the analyzing unit to the stored reference, wherein an amount of the at least one biomarker in the test sample which differs in comparison to the reference is indicative for the presence of an endocrine disease or disorder or wherein an essentially identical amount of the at least one biomarker in the test sample in comparison to the reference is indicative for the absence of an endocrine disease or disorder.

20. A kit for diagnosing an endocrine disease or disorder comprising a detection agent for the at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, or 9 and standards for the at least one biomarker the concentration of which is derived from (i) a subject or a group of subjects known to suffer from an endocrine disease or disorder or a subject or group of subjects which has been brought into contact with at least one compound selected from the group consisting of 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, and Ziprasidone hydrochloride or derived (ii) from a subject or a group of subjects known to not suffer from an endocrine disease or disorder or a subject or group of subjects which has not been brought into contact with at least one compound selected from the group consisting of 17-alpha-Ethynylestradiol, 17-alpha-Methyltestosterone, ACTH, Bromocriptine Mesylate, Buserelin acetate s.c., Cabergoline, Clofibrate, Cyproteron Acetate, Dexamethasone, Diethanolamine, Diethylstilboestrol dipropionate, Epoxiconazole, Fenarimol, Fluoroglycofen-ethyl, Fluoxetine hydrochloride, Flutamide, Formestane, Genistein, Glipizide, Ifosfamide, Iprodione, Leuprolide acetate salt, Medroxyprogesterone acetat, Metazachlor, Methimazole, Mibolerone, Mifepristone, Naphthylisothiocyanate, Norethindrone acetate, Pioglitazone hydrochloride, Raloxifene Hydrochloride, Risperidone, Rosiglitazone maleate, Streptozotocin, Tamoxifen, Trenbolone, Triticonazole, Vinclozolin, and Ziprasidone hydrochloride.
